(19) United States
(12) Patent Application Publication
(10) Pub. No.: US 2024/0143672 A1
(43) Pub. Date: May 2, 2024

(54) EPHEMERAL SOCIAL NETWORKS

(71) Applicant: OnKai Inc., Lewes, DE (US)

(72) Inventors: Daniel Steenstra, Blijham (NL); Noam Smetana, Tel-Aviv (IL); Amikam Levanon, Tel-Aviv (IL); Deborah Bahrani, Advance, NC (US); Charlyn Belluzzo, San Francisco, CA (US)

(21) Appl. No.: 18/404,583

(22) Filed: Jan. 4, 2024

Related U.S. Application Data
(63) Continuation of application No. PCT/US2022/073410, filed on Jul. 5, 2022.
(60) Provisional application No. 63/218,610, filed on Jul. 6, 2021.

(51) Int. Cl. G06F 16/951; G06F 40/30; G06Q 10/0631; G06Q 50/00; G16H 10/20

(52) U.S. Cl. CPC G06F 16/951; G06F 40/30; G06Q 10/063112; G06Q 50/01; G16H 10/20

(57) ABSTRACT

Consistent with disclosed embodiments, systems, methods, and computer readable media include instructions for forming ephemeral social clusters. Embodiments may include a processor to scrape the internet for commonality data identifying a plurality of entities, performing electronic semantic analysis on scraped commonality data to identify a subset of the plurality of entities having at least one specific overlapping interest. Electronic communications may be transmitted to at least some entities and electronic responses may be received. An interest group may be generated based on the responses and defined by the overlapping interest. The processor may receive electronic data from a plurality of different sources to monitor the overlapping interest and redefine the interest group based on the data.

### CROSS REFERENCE TO RELATED APPLICATION

This application claims the benefit of priority of U.S. Provisional Application No. 63/218,610, filed Jul. 6, 2021. The foregoing application is incorporated herein by reference in its entirety.

### BACKGROUND

### Technical Field

The present disclosure relates generally to the automated establishment of digital interactions between network-connected devices of professional entities. More specifically, the present disclosure relates to systems, methods, and devices for identifying technological requirements and facilitating collaboration between entities to address those requirements.

### Background Information

The disclosed methods and systems are directed to providing new and unconventional methods of establishing, monitoring, facilitating, and tracking collaborative relationships for resolving unmet technological needs. For example, in the health care industry, ‘rigid’ healthcare systems have evolved to serve people in hospital centers and to focus on system targets rather than patient outcomes. These systems, however, may not effectively capture needs that arise in underserved industries or identify entities that can work toward addressing these needs. For example, in remote communities, health care staff may suffer from lack of resources that could help them be more effective. Other industries may similarly experience a lack of knowledge or resource sharing among professionals. Therefore, there is a need for systems, methods and computer readable media for capturing unmet technological needs, mapping entities and other resources associated with the needs, and facilitating collaboration in resolving these technological needs.

### SUMMARY

Embodiments consistent with the present disclosure provide systems and methods for analyzing and managing unmet technological needs.

According to some disclosed embodiments, systems, methods, and non-transitory computer-readable media are used for identifying unmet technological needs. They may involve maintaining a data structure containing information about a plurality of unmet technological needs; receiving a query; identifying in the data structure a subset of the plurality of unmet technological needs associated with the query; receiving a selection of a particular unmet technological need from the subset of the plurality of unmet technological needs; receiving a request to identify an extent of the particular unmet technological need; scraping the internet to identify a plurality of sources containing information relating to the particular unmet technological need; performing semantic analysis on the plurality of scraped sources to determine in each scraped source information characterizing an extent of the particular unmet technological need; aggregating the characterization information from each scraped source; analyzing the aggregated characterization information to quantify a number of beneficiaries sharing the particular unmet technological need; and outputting for presentation the quantification in association with an indication of the particular unmet technological need.

According to some disclosed embodiments, systems, methods, and non-transitory computer-readable media involve identifying solutions to unmet technological needs, including receiving an indication of an unmet technological need of at least one entity, electronically extracting data from a data source to identify a plurality of requirements for fulfilling the unmet technological need and performing a first scraping of the internet to identify at least one solution satisfying at least one of the plurality of requirements. Some disclosed embodiments also involve performing a second scraping of the internet to identify at least one proof of concept for the identified at least one solution, a degree of safety associated with the identified at least one solution, an economic feasibility associated with the identified at least one solution, and a commercial applicability associated with the identified at least one solution. The information from the second scraping may then be analyzed to thereby recommend implementation of at least one specific solution from the identified at least one solution.

According to some disclosed embodiments systems, methods, and computer readable media are used for forming ephemeral social clusters. They may involve scraping the internet for commonality data identifying a plurality of entities associated with commonality; performing electronic semantic analysis on the scraped commonality data to identify a subset of the plurality of entities having at least one specific overlapping interest in contributing to a common purpose; transmitting electronic communications to at least some entities of the subset of the plurality of entities; receiving electronic responses to at least some of the electronic communications; based on the received responses, generating an interest group defined by the at least one specific overlapping interest; causing the interest group to be stored in memory, wherein the stored interest group includes information identifying selected entities from the subset of the plurality of entities; receiving electronic data from a plurality of differing sources to thereby monitor the at least one specific overlapping interest of each of the selected entities and to determine that the overlapping interest is reduced for a specific one of the selected entities; and redefining the interest group to exclude the specific one of the selected entities following a determination that the at least one specific overlapping interest of the specific one of the selected entities is reduced.

According to some disclosed embodiments systems, methods, and computer readable media are used for associating entities with unestablished relationships in order to induce collaboration. They may involve receiving information identifying a current unmet technological need; accessing an electronic data source to identify, in relation to the current unmet technological need, a plurality of skill sets of a plurality of entities, and a plurality of technological need-related roles of the plurality of entities; scraping the internet to identify at least one prior solution for resolving a previous unmet technological need, the previous unmet technological need being related to the current unmet technological need, and a plurality of prior roles and a plurality of prior skills associated with the at least one prior solution; generating at least one collaboration rule based on the identified prior solution, the identified plurality of prior roles, and the identified plurality of prior skills; applying the at least one collaboration rule to the electronic data source to identify, based on the plurality of skill sets and the plurality of technological need-related roles of the plurality of entities, at least two entities of the plurality of entities projected to have an ability to collaborate in order to satisfy the current unmet technological need; and outputting an identification of the at least two entities in an associative manner in connection with the current unmet technological need.

According to some disclosed embodiments systems, methods, and computer readable media are used for internet-based smart contracting and collaboration. They may involve accessing terms of a collaborative smart contract between at least two previously unconnected entities, the at least two previously unconnected entities including a first entity and a second entity respectively located in a first venue and in a second venue, the collaborative smart contract defining a plurality of success criteria for resolving an unmet technological need shared by the first entity and the second entity; remotely monitoring, over at least one network, activity of the first entity in the first venue to track progress of the first entity in satisfying a first portion of the success criteria associated with the first entity; remotely monitoring, over the at least one network, activity of the second entity in the second venue, to track progress in satisfying a second portion of the success criteria associated with the second entity; based at least on the remote monitoring of the activity of the first entity and the activity of the second entity, determining that all of the success criteria are satisfied; and then performing sematic analysis to identify a plurality of additional entities who share the unmet technological need; and transmitting to the plurality of additional entities an indication that the success criteria are satisfied, and an opportunity related to the satisfied success criteria. Disclosed embodiments may also include executing activities based on pre-defined rules (e.g., automatically releasing payment)

According to some disclosed embodiments systems, methods, and computer readable media are used for mapping a series of related unmet technological needs. They may involve receiving first onboarding information from a plurality of first entities, the first onboarding information including information indicative of a plurality of first unmet technological needs; establishing a map of the first unmet technological needs, the map including a plurality of nodes representing the first unmet technological needs; establishing pathways between the plurality of nodes, wherein the pathways define relationships between nodes based on similarities between corresponding unmet technological needs; associating a particular entity of the plurality of first entities with a first particular node of the plurality of nodes; receiving first content associated with the first particular node, the first content being available to the particular entity by virtue of the particular entity being associated with the first particular node; receiving second onboarding information from a plurality of second entities, the second onboarding information including information indicative of a plurality of second unmet technological needs; altering the map based on the second onboarding information, wherein altering the map includes establishing a second particular node of the plurality of second unmet technological needs, establishing a pathway between the first particular node and the second particular node, and moving the particular entity from the first particular node to the second particular node, wherein the second particular node is associated with an unmet technological need different from the unmet technological need associated with the first particular node; and receiving second content within the second particular node, the second content being available to the particular entity by virtue of the particular entity being associated with the second particular node.

Consistent with other disclosed embodiments, non-transitory computer readable storage media may store program instructions, which are executed by at least one processor and perform any of the methods described herein.

The foregoing general description and the following detailed description are exemplary and explanatory only and are not restrictive of the claims.

### BRIEF DESCRIPTION OF THE DRAWINGS

The accompanying drawings, which are incorporated in and constitute a part of this disclosure, illustrate various disclosed embodiments. In the drawings:

FIG. 1 is a diagrammatic representation of an exemplary system consistent with some disclosed embodiments.

FIG. 2A is a block diagram showing an example server, consistent with some disclosed embodiments.

FIG. 2B is a block diagram showing an example computing device, consistent with some disclosed embodiments.

FIGS. 3A and 3B are illustrations of example user interfaces, consistent with some disclosed embodiments.

FIG. 4A illustrates an example of a data structure for storing information associated with an unmet technological need, consistent with some disclosed embodiments.

FIGS. 4B and 4C illustrate example user interfaces for obtaining information about an unmet technological need, consistent with some disclosed embodiments.

FIG. 4D illustrates an example user interface through which a user may submit a query consistent with some disclosed embodiments.

FIG. 4E illustrates an example user interface for displaying a quantification of a number of beneficiaries sharing a particular unmet technological need, consistent with some disclosed embodiments.

FIG. 5 is a flowchart showing an example process for identifying unmet technological needs, consistent with some disclosed embodiments.

FIG. 6A is a block diagram illustrating an example process for extracting data from a data source to identify a plurality of requirements, consistent with some disclosed embodiments.

FIG. 6B is a block diagram illustrating an example, process for recommending a solution based on scraped information, consistent with some disclosed embodiments.

FIG. 6C illustrates an example interface that may be presented to a user for recommending implementation of a solution, consistent with some disclosed embodiments.

FIG. 7 is a flowchart showing an example process for identifying solutions to unmet technological needs, consistent with some disclosed embodiments.

FIG. 8A is a diagrammatic representation of an exemplary system transmitting electronic communications to at least some entities.

FIG. 8B is a diagrammatic representation of an exemplary system receiving electronic responses to at least some of the electronic communications.

FIG. 8C is a diagrammatic representation of generating an interest group based on the received responses.

FIG. 9 is a flow diagram of an exemplary method that may be executed by a processor to perform operations for forming ephemeral social clusters.

FIG. 10 is a diagrammatic representation of an exemplary system for scraping the internet to identify prior solutions, roles, and skills, consistent with the disclosed embodiments.

FIG. 11 is flow diagram of an exemplary method that may be executed by a processor to perform operations for associating entities with unestablished relationships in order to induce collaboration.

FIG. 12 is a diagrammatic representation of an exemplary system for internet-based smart contracting and collaboration, consistent with the disclosed embodiments.

FIG. 13 is flow diagram of an exemplary method that may be executed by a computer for internet-based smart contracting and collaboration, consistent with the disclosed embodiments.

FIG. 14A is a diagrammatic representation of an exemplary map of related unmet technological needs, consistent with the disclosed embodiments.

FIG. 14B is a diagrammatic representation of an exemplary altered map of related unmet technological needs, consistent with the disclosed embodiments.

FIG. 15 is flow diagram of an exemplary method that may be executed by a computer for mapping a series of related unmet technological needs, consistent with the disclosed embodiments.

### DETAILED DESCRIPTION

Unless specifically stated otherwise, throughout the specification discussions utilizing terms such as “processing”, “calculating”, “computing”, “determining”, “generating”, “setting”, “configuring”, “selecting”, “defining”, “applying”, “obtaining”, “monitoring”, “providing”, “identifying”, “segmenting”, “classifying”, “analyzing”, “associating”, “extracting”, “storing”, “receiving”, “transmitting”, or the like, include actions and/or processes of a computer that manipulate and/or transform data into other data, the data represented as physical quantities, for example such as electronic quantities, and/or the data representing physical objects. The terms “computer”, “processor”, “controller”, “processing unit”, “computing unit”, and “processing module” should be expansively construed to cover any kind of electronic device, component or unit with data processing capabilities, including, by way of non-limiting example, a personal computer, a wearable computer, smart glasses, a tablet, a smartphone, a server, a computing system, a cloud computing platform, a communication device, a processor (for example, digital signal processor (DSP), an image signal processor (ISR), a microcontroller, a field programmable gate array (FPGA), an application specific integrated circuit (ASIC), a central processing unit (CPA), a graphics processing unit (GPU), a visual processing unit (VPU), and so on), possibly with embedded memory, a single core processor, a multi core processor, a core within a processor, any other electronic computing device, or any combination of the above.

In the following detailed description, numerous specific details are set forth in order to provide a thorough understanding of the disclosed example embodiments. However, it will be understood by those skilled in the art that the principles of the example embodiments may be practiced without every specific detail. Well-known methods, procedures, and components have not been described in detail so as not to obscure the principles of the example embodiments. Unless explicitly stated, the example methods and processes described herein are not constrained to a particular order or sequence, or constrained to a particular system configuration. Additionally, some of the described embodiments or elements thereof can occur or be performed simultaneously, at the same point in time, or sequentially.

Throughout, this disclosure mentions “disclosed embodiments,” which refer to examples of inventive ideas, concepts, and/or manifestations described herein. Many related and unrelated embodiments are described throughout this disclosure. The fact that some “disclosed embodiments” are described as exhibiting a feature or characteristic does not mean that other disclosed embodiments necessarily share that feature or characteristic.

This disclosure employs open-ended permissive language, indicating for example, that some embodiments “may” employ, involve, or include specific features. The use of the term “may” and other open-ended terminology is intended to indicate that although not every embodiment may employ the specific disclosed feature, at least one embodiment employs the specific disclosed feature.

Some aspects of the present disclosure relate to the identification, monitoring, management, and resolution of technological needs. As used herein, a technological need may refer to a circumstance or set of circumstances that require some course of action that is technical in nature. For example, a technological need may be a need that is related to endeavors that are scientific, biological, engineering-related, industrial, occupational, professional, scholarly, vocational, health-related, medical, chemical, environmental, security-related, mathematical, or otherwise technical in nature. In some embodiments, a technological need may be “unmet” in that one or more aspects of the technological need may still require a course of action.

Technological needs may arise in a variety of industries, any of which may be addressed according to the disclosed embodiments. By way of non-limiting example, technological needs may arise in the medical field. The disclosed embodiments may be implemented to identify and address such needs. As one example, a technological need may be a need to complete a clinical trial and may be unmet in that the clinical trial has not yet been completed. As described in further detail below, the disclosed embodiments may be implemented to identify this need and to assist with satisfying or meeting the need. In the example of a clinical trial, this may include identifying one or more entities for collaborating to complete the clinical trial. This may include identifying equipment needed for testing or performing other medical procedures, medical clinics able to host a trial, healthcare providers or other experts able to conduct various aspects of the clinical trial, patients or other participants in the trial (e.g., a cohort, etc.), or the like. The disclosed embodiments may further manage relationships between these various entities, which may include identifying the identities, querying the identities to determine an interest or availability, tracking a progress of the entities, facilitating communications between the entities, rewarding the entities, or the like.

Further, a technological need can refer to needs of a variety of magnitudes or degrees. For example, a technological need may refer to an ultimate goal shared by one or more entities. In the example of a clinical trial, this ultimate goal may refer to completion of the trial. Alternatively or additionally, an unmet technological need may refer to various levels of sub-needs or activities. Continuing with the clinical trial example, a technological need may include selecting a cohort of patients for the trial, identifying a piece of equipment or other resource needed for the trial, transporting a patient from one location to another in association with conducting the clinical trial, obtaining a signature on a form, or a various other degrees or types of technological needs. Accordingly, some disclosed embodiments may be applicable to an overall goal or to various discrete tasks or activities towards meeting the goal, and may be implemented multiple times in association with a particular goal.

While a clinical trial is provided by way of example throughout the present disclosure, it is to be understood that technological needs may encompass a wide variety of needs. For example, even within the medical field, technological needs may arise in association with treatment of a particular medical condition experienced by one or more patients, diagnosis of certain symptoms experienced by one or more patients, finding diagnostic or treatment equipment or facilities for one or more patients, finding medical professionals trained with particular skills, building patient cohorts of patients sharing a particular characteristic, or a wide variety of other technological needs. Further, one skilled in the art would appreciate that the disclosed techniques for identifying and managing unmet technological needs may be implemented in a nearly endless number of other industries. For example, this may include technological needs associated with computer programming, engineering or design, video game development, ecommerce, consumer electronics, government or infrastructure, telecommunications, artificial intelligence, consulting, entertainment and the arts (e.g., music, motion pictures, sculpture, choreography, etc.), marketing, education, agriculture, finance, insurance, energy, internet-of-things, transportation, fashion, sports, space exploration, or any other industry in which unmet technological needs may arise.

Systems consistent with some disclosed embodiments may include one or more servers configured to communicate with various computing devices or entities. As used herein, a server may be any form of computing device capable of accessing data through a network and processing the data consistent with the disclosed embodiments. In some embodiments, the server may include a single computing device, such as a server rack. In other embodiments, the remote server may include multiple computing devices, such as a server farm or server cluster. The remote server may also include network appliances, mobile servers, cloud-based server platforms, or any other form of central computing platform. Various example remote servers are described in greater detail below.

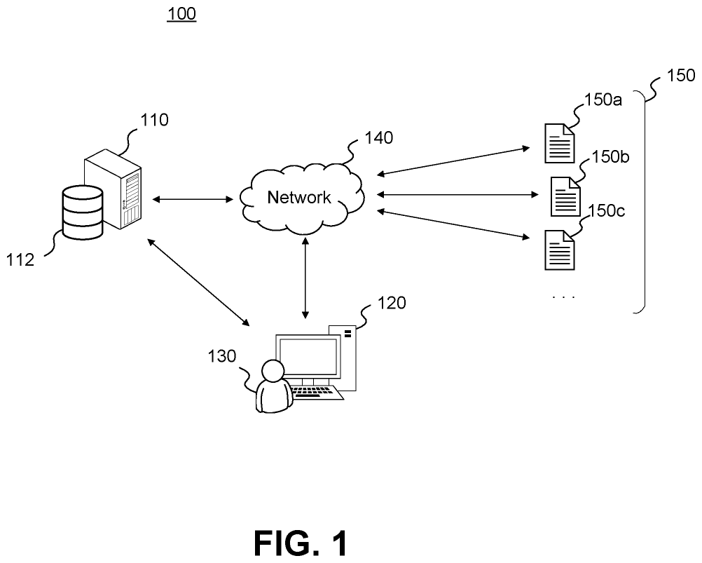

FIG. 1 illustrates an example system 100 for identifying and managing technological needs, consistent with some disclosed embodiments. As shown in FIG. 1, system 100 may include a server 110. Server 110 may be any form of one or more computing devices for accessing data, processing data, storing data, and/or transmitting data to various other entities or computing devices. For example, this may include data associated with an unmet technological need, as described above. A computing device may refer to any structure that includes at least one processor. As used herein, “at least one processor” may constitute any physical device or group of devices having electric circuitry that performs a logic operation on an input or inputs. For example, the at least one processor may include one or more integrated circuits (IC), including application-specific integrated circuit (ASIC), microchips, microcontrollers, microprocessors, all or part of a central processing unit (CPU), graphics processing unit (GPU), digital signal processor (DSP), field-programmable gate array (FPGA), server, virtual server, or other circuits suitable for executing instructions or performing logic operations. The instructions executed by at least one processor may, for example, be pre-loaded into a memory integrated with or embedded into the controller or may be stored in a separate memory. The memory may include a Random Access Memory (RAM), a Read-Only Memory (ROM), a hard disk, an optical disk, a magnetic medium, a flash memory, other permanent, fixed, or volatile memory, or any other mechanism capable of storing instructions. In some embodiments, the at least one processor may include more than one processor. Each processor may have a similar construction or the processors may be of differing constructions that are electrically connected or disconnected from each other. For example, the processors may be separate circuits or integrated in a single circuit. When more than one processor is used, the processors may be configured to operate independently or collaboratively, and may be co-located or located remotely from each other. The processors may be coupled electrically, magnetically, optically, acoustically, mechanically or by other means that permit them to interact.

In some embodiments, server 110 may access at least one database, such as database 112. As used herein, a “database” may be construed synonymously with a “data structure” and may include any collection or arrangement of data values and relationships among them, regardless of structure. For example, a database may refer to a tangible storage device, e.g., a hard disk, used as a database, or to an intangible storage unit, e.g., an electronic database. As used herein, any data structure may constitute a database. The data contained within a data structure may be stored linearly, horizontally, hierarchically, relationally, non-relationally, uni-dimensionally, multidimensionally, operationally, in an ordered manner, in an unordered manner, in an object-oriented manner, in a centralized manner, in a decentralized manner, in a distributed manner, in a custom manner, or in any manner enabling data access. By way of non-limiting examples, data structures may include an array, an associative array, a linked list, a binary tree, a balanced tree, a heap, a stack, a queue, a set, a hash table, a record, a tagged union, ER model, and a graph. For example, a data structure may include an XML database, an RDBMS database, an SQL database or NoSQL alternatives for data storage/search such as, for example, MongoDB, Redis, Couchbase, Datastax Enterprise Graph, Elastic Search, Splunk, Solr, Cassandra, Amazon DynamoDB, Scylla, HBase, and Neo4J. A data structure may be a component of the disclosed system or a remote computing component (e.g., a cloud-based data structure). Data in the data structure may be stored in contiguous or non-contiguous memory. Moreover, a data structure, as used herein, does not require information to be co-located. It may be distributed across multiple servers, for example, that may be owned or operated by the same or different entities. Thus, the term “data structure” as used herein in the singular is inclusive of plural data structures.

In some embodiments, server 110 may communication with one or more computing devices, such as computing device 120. Computing device 120 may include any device that may be used for performing conducting various operations associated with a technical need. Accordingly, computing device 120 may include various forms of computer-based devices, such as a workstation or personal computer (e.g., a desktop or laptop computer), a mobile device (e.g., a mobile phone or tablet), a wearable device (e.g., a smart watch, smart jewelry, implantable device, fitness tracker, smart clothing, head-mounted display, etc.), an IoT device (e.g., smart home devices, industrial devices, etc.), or any other device that may be capable of receiving, storing, processing, or transmitting data. In some embodiments, computing device 120 may be a virtual machine (e.g., based on AWS™, Azure™, IBM Cloud™, etc.), container instance (e.g., Docker™ container, Java™ container, Windows Server™ container, etc.), or other virtualized instance.

In some embodiments, computing device 120 may be associated with an entity 130. As used herein, an entity may refer to any distinct or independent existence. For example, an entity be an individual, a user, a device, an account, an application, a process, a service, a facility, a piece of equipment, an organization, or any other form of object, article or person. Alternatively or additionally, an entity may be a group of two or more components (e.g., individuals, users, devices, accounts, etc.) forming a single entity. In some embodiments, entity 130 may be associated with a goal or unmet technological need. In some embodiments, entity 130 may be an individual having one or more skills or other attributes that may enable the individual to contribute to meeting a goal or resolving an unmet need, as described in further detail herein.

Consistent with the disclosed embodiments, the various components may communicate over a network 140. Such communications may take place across various types of networks, such as the Internet, a wired Wide Area Network (WAN), a wired Local Area Network (LAN), a wireless WAN (e.g., WiMAX), a wireless LAN (e.g., IEEE 802.11, etc.), a mesh network, a mobile/cellular network, an enterprise or private data network, a storage area network, a virtual private network using a public network, a nearfield communications technique (e.g., Bluetooth®, infrared, etc.), or any other type of network for facilitating communications. In some embodiments, the communications may take place across two or more of these forms of networks and protocols. While system 100 is shown as a network-based environment, it is understood that the disclosed systems and methods may also be used in a localized system, with one or more of the components communicating directly with each other. As shown in FIG. 1, in some embodiments, server 110 may be configured to communicate directly with computing device 120 (e.g., without an intermediate device). For example, server 110 may be configured to transmit requests, queries, data, or any other information described herein directly to computing device 120 or vice versa.

According to some embodiments, server 110, computing device 120, or both, may be configured to access information from at least one data source 150, which may include sources 150a, 150b, and 150c. Data source 150 may include any form of electronic, network-based resource storing data accessible through network 140. In some embodiments, data source 150 may be a database, as described above. For example, data source 150 may include one or more of a news database (the website of The New York Times or the Associated Press (AP) RSS feed), API's such as those defined for the Centers for Disease Control and Prevention (CDC), a database of entity information (e.g., addresses, phone numbers, other contact information, skill or role information, etc.), social media databases, encyclopedia databases, a journal or publication database (e.g., a collection of scholarly or professional publications), a government databases (e.g., the Taxonomy Database provided by the National Center for Biotechnology Information), or various other online databases. In some embodiments, data source 150 may include information hosted on a webpage. Accordingly, data source 150 may include a web server hosting information accessible over the Internet, or the like.

In some embodiments, data source 150 may include a plurality of differing data sources or types of data sources. For example, source 150a may comprise a local database, source 150b may comprise a news database, and source 150c may comprise a research journal database. In certain aspects, one or more sources may be operably connected together (e.g., sources 150b and 150c) and/or one or more sources may be operably independent (like source 150a, for example). In some embodiments, server 110 may be configured to aggregate data from multiple data sources. This may include analyzing, combining, comparing, filtering, extracting, summarizing, or performing various other processes on the accessed data.

In some embodiments, the disclosed embodiments may include accessing information through web scraping techniques, which may also be referred to as scraping the Internet. Accordingly, network 140 may include, at least in part, the Internet, and server 110 may perform a scraping technique to receive information from data source 150. As used herein, “scraping” or “scraping the Internet” may include any manner of data aggregation, that involves a machine, including but not limited to crawling across websites, identifying links and changes to websites, data transfer through API's, FTP's, GUI, direct database connections through parsing and extraction of website pages, or any other suitable form of machine-associated data acquisition. In certain aspects, server 110 may execute one or more applications configured to function as web scrapers. A web scraper may include a web crawler and an extraction bot. A web crawler may be configured to find, index, and/or fetch web pages and documents. An extraction bot may be configured to copy the crawled data to server 110 or may be configured to process the crawled data and copy the processed data to server 110. For example, the bot may parse, search, reformat, etc., the crawled data before copying it.

Information scraped from the plurality of sources may include web pages (e.g., HTML documents), documents represented in various file formats (e.g., pdf, txt, rtf, doc, docx, ppt, pptx, opt, png, tiff, png, jpeg, odt, ods, fbx, xml, JSON, etc.), decentralized ledgers such as blockchain ledgers, node wallets, or various other data that may be available through the Internet. The web scraper may be configured to modify one or more types of scraped data (e.g., HTML) to one or more other types of scraped data (e.g., txt) before copying it to server 110. In some embodiments, this may include converting the data to a standardized format so that it may be stored and analyzed in a consistent manner in server 110. As another example, this may include performing an optical character recognition (OCR) technique to convert handwritten or printed text into machine-encoded text.

The web scraper may run continuously, near continuously, periodically at scheduled collection intervals (e.g., every hour, every two hours, etc.), on-demand based on a request (e.g., entity 130 may send a request to server 110 that initiates a scraping session), or based on various other trigger events described herein. In some embodiments, the web scraper may run at different intervals for different sources. For example, the web scraper may run every hour for data source 150a and run every two hours for data source 150b. This may allow the web scraper to account for varying excess traffic limits and/or to account for varying bandwidth limits that may result in suboptimal performance or crashes of a source. In some embodiments, web scraping may be performed at a very large scale such that large volumes of data can be accessed in short timeframes, thereby providing improved efficiency over manually accessing data.

Embodiments consistent with the present disclosure may include performing a semantic analysis. As used herein, semantic analysis may refer to any form of analysis or processing of natural language (in text form) to draw meaning and/or context. Semantic analysis may include an electronic semantic analysis performed by a processor using various semantic analysis algorithms. Semantic analysis may include relating syntactic structures from phrases, clauses, sentences, and paragraphs to a writing as a whole to determine their language-independent meanings. In some embodiments, semantic analysis may include parsing elements of text and assigning each a grammatical role. The structure may then be analyzed to remove ambiguity from any word with multiple meanings. As one example, semantic analysis may include analyzing sentences and sequences of words by using a set of rules, principles, and processes in order to determine a skill associated with an entity, as described below. In some embodiments a semantic analysis may be performed on data scraped from the internet to derive meaning from the scraped data, which may be used to perform various analytics described herein.

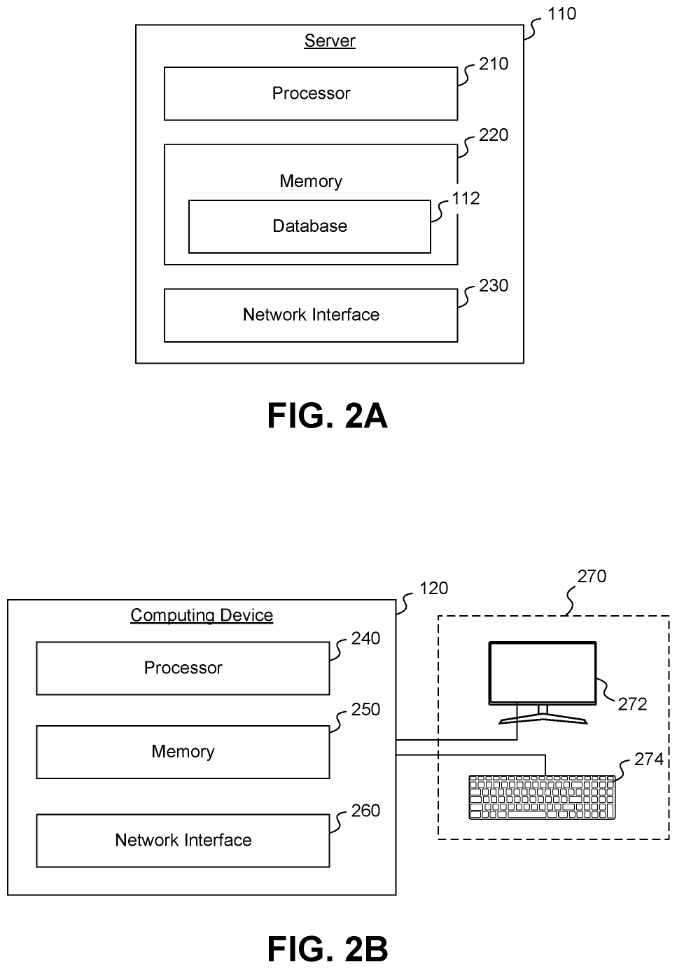

FIG. 2A is a block diagram illustrating an example server 110, consistent with some disclosed embodiments. As described above, server 110 may be a computing device and may include one or more dedicated processors and/or memories. For example, server 110 may include at least one processor, more generally referred to as processor 210, a memory (or multiple memories) 220, a network interface (or multiple network interfaces) 230, as shown in FIG. 2A. As indicated above, in some embodiments, server 110 may be a rack of multiple servers. Accordingly, server 110 may include multiple instances of the example server shown in FIG. 2A.

Processor 210 may take the form of, but is not limited to, a microprocessor, embedded processor, or the like, may be integrated in a system on a chip (SoC), or more take the form of any processor described earlier. Furthermore, according to some embodiments, the processor 210 may be from the family of processors manufactured by Intel®, AMD®, Qualcomm®, Apple®, NVIDIA®, or the like. Processor 210 may also be based on an ARM architecture, a mobile processor, or a graphics processing unit, etc. The disclosed embodiments are not limited to any type of processor included in server 110. In some embodiments, processor 210 may refer to multiple processors.

Memory 220 may include one or more storage devices configured to store instructions used by the processor 210 to perform functions related to the disclosed embodiments. Memory 220 may be configured to store software instructions, such as programs, that perform one or more operations when executed by the processor 210 to perform the various functions or methods described herein. The disclosed embodiments are not limited to particular software programs or devices configured to perform dedicated tasks. For example, memory 220 may store a single program, such as a user-level application, that performs the functions of the disclosed embodiments, or may include multiple software programs Additionally, the processor 210 may in some embodiments execute one or more programs (or portions thereof) remotely located from server 110. Furthermore, the memory 220 may include one or more storage devices configured to store data for use by the programs In some embodiments, memory 220 may include a local database 112, as described in further detail above.

Network interface 230 may include one or more network adaptors or communication devices and/or interfaces (e.g., WiFi®, Bluetooth®, RFID, NFC, RF, infrared, Ethernet, etc.) to communicate with other machines and devices, such as with other components of system 100 through network 140. For example, server 110 may use a network adaptor to receive and transmit information associated with technological needs within system 100.

FIG. 2B is a block diagram illustrating an example computing device 120, consistent with some disclosed embodiments. Computing device 120 may also include one or more dedicated processors and/or memories, similar to server 110. For example, computing device 120 may include at least one processor 240, a memory (or multiple memories) 250, a network interface (or multiple network interfaces) 260, and/or one or more input/output (I/O) devices 240, as shown in FIG. 2B. Processor 240, memory 250, and network interface 260 may be similar to processor 210, memory 220 and network interface 230, described above. Accordingly, any details, examples, or embodiments described above with respect to processor 210, memory 220 and network interface 230 may equally apply to processor 240, memory 250, and network interface 260.

For example, processor 240 may take the form of, but is not limited to, a microprocessor, embedded processor, or the like, may be integrated in a system on a chip (SoC), or more take the form of any processor described earlier. The disclosed embodiments are not limited to any type of processor included in computing device 120 and processor 240 may refer to multiple processors. Memory 250 may include one or more storage devices configured to store instructions used by the processor 240 to perform functions related to the disclosed embodiments. Memory 250 may be configured to store software instructions, such as programs, that perform one or more operations when executed by the processor 240 to perform the various functions or methods described herein. Network interface 260 may include one or more network adaptors or communication devices and/or interfaces (e.g., WiFi®, Bluetooth®, RFID, NFC, RF, infrared, Ethernet, etc.) to communicate with other machines and devices, such as with other components of system 100 (including server 110) through network 140.

I/O devices 270 may include one or more interface devices for interfacing with a user of server 110. For example, I/O devices 270 may include a display 272 configured to display various information to a user, such as entity 130. In some embodiments, display 272 may be configured to present one or more graphical user interfaces to a user and may receive information through the graphical user interface. In some embodiments, I/O devices 270 may include a keyboard 274 or other device through which a user may input information. I/O devices 270 may include various other forms of devices, including but not limited to lights or other indicators, a touchscreen, a keypad, a mouse, a trackball, a touch pad, a stylus, buttons, switches, dials, motion sensors, microphones, video capturing devices, or any other user interface device, configured to allow a user to interact with computing device 120. Although I/O devices 270 are illustrated as external or separate components from computing device 120 by way of example, it is to be understood that computing device 120 may be defined to include I/O devices 270. In some embodiments, I/O devices 270 may be integral to computing device 120. For example, in embodiments where computing device 120 includes a mobile device such as a phone or tablet computer, I/O devices 270 may be integral to computing device 120.

Some disclosed embodiments may include presenting various user interfaces to receive information from a user. For example, this may include displaying one or more graphical user interfaces on display 272 and receiving a user input through keyboard 274 or various other forms of I/O devices. Consistent with the present disclosure, the user inputs may be used to define or provide various information, including but not limited to technological needs, characterization information, economic impact or feasibility information, proof of concept information, commercial applicability information, solutions to technological needs, beneficiary information, degree of impact information, roles and skills, contact information, demographic data (e.g., age, gender, ethnicity, etc.) or various other forms of information described herein.

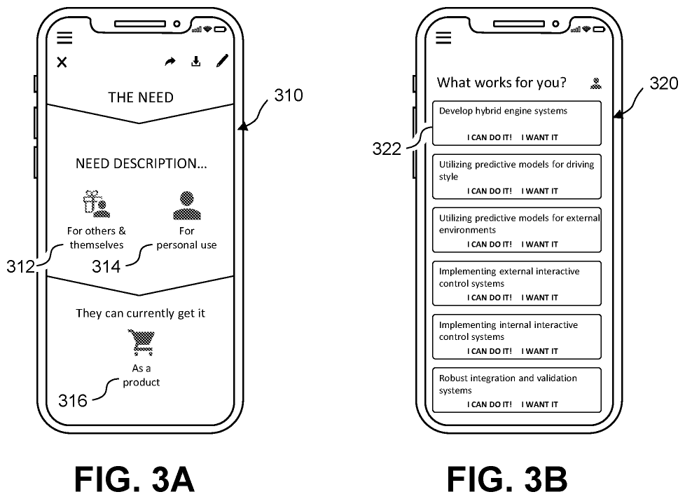

FIG. 3A illustrates an example user interface 310 that may be presented to a user for acquiring information about a technical need, consistent with some disclosed embodiments. For example, user interface 310 may be presented to a user via computing device 120. User interface 310 may include various interactive elements 312, 314, and 316, which may allow a user to select various properties of an unmet technological need. In this example, interactive element 312 may allow a user to specify that a technological need applies to others and themselves (e.g., a healthcare professional specifying the needs of his or her patients, a business specifying the needs of their clients, a politician specifying the needs of his or her constituents, etc.). Alternatively or additionally, a user may select interactive element 314 to specify that a technological need applies to the user personally (e.g., a need for a ride to a destination, a need for a healthcare service, a need for maintenance on a piece of equipment, a home repair, etc.). User interface 310 may further include one or more elements 316 to allow a user to specify how the technological need is currently satisfied for the user or others. In this example, a user may specify through element 316 that the need is currently offered as a product. In some embodiments, user interface 310 may allow a user to specify solutions to the technological need that are offered as a service, as a software platform, through collaborative efforts, or various other forms of solutions.

FIG. 3B illustrates another example user interface 320 that may be presented to a user for acquiring information about abilities and/or desires of an entity, consistent with some disclosed embodiments. For example, user interface 320 may be used to gather information about an entity to define a set of skills associated with an entity, which may be used to assess an ability of a user to contribute to achieving a goal, as described further below. Alternatively or additionally, user interface 320 may be used to gather information defining technological needs of an entity, which may be used to classify and address the needs of the entity. In the example shown in FIG. 3B, user interface 320 may include a series of “card” elements presenting example abilities and desires. For example, card element 322 may refer to a skill for developing hybrid engine systems, through which a user may specify they possess the skill, or whether they are in need of this skill (e.g., as a service, product, etc.). While various user interfaces are provided throughout the present disclosure, it is to be understood that the various elements, layouts, and information presented therein are shown by way of example. One skilled in the art would recognize that various other forms of user interfaces may be implemented, depending on the particular application or based on individual preferences. For example, while user interface 320 is presented as a series of cards, one skilled in the art would recognize that similar information may be acquired through various other user interface layouts and controls. Accordingly, any of the various user interfaces presented herein may include various forms of buttons, text input fields, radio buttons, checkboxes, dropdown lists or menus, links, breadcrumbs, timelines, tabs, links, tree panes, menus, accordion controls, icons, tooltips, alerts, pop-ups, touchscreen interfaces, or any other form of element for inputting and/or displaying information.

In some embodiments, the various techniques described herein may include application of one or more trained machine learning algorithms. These machine learning algorithms (also referred to as machine learning models in the present disclosure) may be trained using training examples to perform particular functions (including both supervised and/or unsupervised), as described more specifically in the various examples herein. Some non-limiting examples of such machine learning algorithms may include classification algorithms, data regressions algorithms, image segmentation algorithms, visual detection algorithms (such as object detectors, face detectors, person detectors, motion detectors, edge detectors, etc.), visual recognition algorithms (such as face recognition, person recognition, object recognition, etc.), speech recognition algorithms, mathematical embedding algorithms, natural language processing algorithms, support vector machines, random forests, nearest neighbors algorithms, deep learning algorithms, artificial neural network algorithms, convolutional neural network algorithms, recursive neural network algorithms, linear machine learning models, non-linear machine learning models, ensemble algorithms, and so forth. For example, a trained machine learning algorithm may include an inference model, such as a predictive model, a classification model, a regression model, a clustering model, a segmentation model, an artificial neural network (such as a deep neural network, a convolutional neural network, a recursive neural network, etc.), a random forest, a support vector machine, and so forth. In some examples, the training examples may include example inputs together with the desired outputs corresponding to the example inputs. For example, in the context of determining an unmet technological need, the training data may include snippets of text (which may represent text input by a user, text scraped from the internet, etc.) along with classifications of an unmet technological need described in the corresponding snippet. In some embodiments, the training data may include various other information that may be useful for training the model. For example, the training data may include a behavior classification indicating a behavioral response by a user. For example, the behavioral classification may indicate that the text was exposed to the user for a specified duration and was ignored by the user, or similar behavioral classifications that may indicate a behavioral response by a user. Accordingly, the trained model may be configured classify unmet technological needs from other text inputs.

Further, in some examples, training machine learning algorithms using the training examples may generate a trained machine learning algorithm, and the trained machine learning algorithm may be used to estimate outputs for inputs not included in the training examples. In some examples, engineers, scientists, processes and machines that train machine learning algorithms may further use validation examples and/or test examples. For example, validation examples and/or test examples may include example inputs together with the desired outputs corresponding to the example inputs, a trained machine learning algorithm and/or an intermediately trained machine learning algorithm may be used to estimate outputs for the example inputs of the validation examples and/or test examples, the estimated outputs may be compared to the corresponding desired outputs, and the trained machine learning algorithm and/or the intermediately trained machine learning algorithm may be evaluated based on a result of the comparison. In some examples, a machine learning algorithm may have parameters and hyper parameters. For example, the hyper parameters may be set automatically by a process external to the machine learning algorithm (such as a hyper parameter search algorithm), and the parameters of the machine learning algorithm may be set by the machine learning algorithm according to the training examples. In some implementations, the hyper-parameters are set according to the training examples and the validation examples, and the parameters are set according to the training examples and the selected hyper-parameters.

In some embodiments, trained machine learning algorithms (also referred to as trained machine learning models in the present disclosure) may be used to analyze inputs and generate outputs, for example in the cases described below. In some examples, a trained machine learning algorithm may be used as an inference model that when provided with an input generates an inferred output. For example, a trained machine learning algorithm may include a classification algorithm, the input may include a sample, and the inferred output may include a classification of the sample (such as an inferred label, an inferred tag, and so forth). In another example, a trained machine learning algorithm may include a regression model, the input may include a sample, and the inferred output may include an inferred value for the sample. In yet another example, a trained machine learning algorithm may include a clustering model, the input may include a sample, and the inferred output may include an assignment of the sample to at least one cluster. In an additional example, a trained machine learning algorithm may include a classification algorithm, the input may include an image, and the inferred output may include a classification of an item depicted in the image. In yet another example, a trained machine learning algorithm may include a regression model, the input may include an image, and the inferred output may include an inferred value for an item depicted in the image (such as an estimated property of the item, such as size, volume, age of a person depicted in the image, cost of a product depicted in the image, and so forth). In an additional example, a trained machine learning algorithm may include an image segmentation model, the input may include an image, and the inferred output may include a segmentation of the image. In yet another example, a trained machine learning algorithm may include an object detector, the input may include an image, and the inferred output may include one or more detected objects in the image and/or one or more locations of objects within the image. In some examples, the trained machine learning algorithm may include one or more formulas and/or one or more functions and/or one or more rules and/or one or more procedures, the input may be used as input to the formulas and/or functions and/or rules and/or procedures, and the inferred output may be based on the outputs of the formulas and/or functions and/or rules and/or procedures (for example, selecting one of the outputs of the formulas and/or functions and/or rules and/or procedures, using a statistical measure of the outputs of the formulas and/or functions and/or rules and/or procedures, and so forth). In some embodiments, the input may include a sequence of images and the trained machine learning algorithm may infer a behavior of a user or other events captured in the sequence of images. This may be used to determine a behavioral classification of a user, as described above.

In some embodiments, artificial neural networks may be configured to analyze inputs and generate corresponding outputs. Some non-limiting examples of such artificial neural networks may include shallow artificial neural networks, deep artificial neural networks, feedback artificial neural networks, feed forward artificial neural networks, autoencoder artificial neural networks, probabilistic artificial neural networks, time delay artificial neural networks, convolutional artificial neural networks, recurrent artificial neural networks, long short-term memory artificial neural networks, and so forth. In some examples, an artificial neural network may be configured by a user. For example, a structure of the artificial neural network, a type of an artificial neuron of the artificial neural network, a parameter of the artificial neural network (such as a parameter of an artificial neuron of the artificial neural network), and so forth may be selected by a user. In some examples, an artificial neural network may be configured using a machine learning algorithm. For example, a user may select hyper-parameters for the artificial neural network and/or the machine learning algorithm, and the machine learning algorithm may use the hyper-parameters and training examples to determine the parameters of the artificial neural network, for example using back propagation, using gradient descent, using stochastic gradient descent, using mini-batch gradient descent, and so forth. In some examples, an artificial neural network may be created from two or more other artificial neural networks by combining the two or more other artificial neural networks into a single artificial neural network.

As described throughout the present disclosure, the disclosed systems, methods and computer readable media may be implemented to address an unmet technological need. This may include developing specific plans for resolving an unmet technological need, generating dynamic groups or maps of entities and resources for resolving the unmet technological need, managing relationships between entities (e.g., through smart contracts, etc.), or various other techniques. In order to more effectively and efficiently address the unmet technological needs, it may be beneficial to classify or quantify an extent of a particular unmet technological need. For example, this may include quantifying the extent to which an unmet technological need is experienced either globally or within a specified area or population. This may further include quantifying various other factors, such as an economic impact of the unmet technological need, a severity of the unmet technological need, or various other metrics. As an illustrative example, a community may report an unmet technological need associated with a healthcare need, such as a substance abuse issue. The disclosed embodiments may be implemented to determine the incidence and prevalence of this need across a larger population (e.g., within an area of a city, across one or more states, nationally, within a particular ethnic group or other demographic, etc.), the efficacy of current measures (if any) to resolve the unmet technological need, and the quantifiable impact of this unmet technological need. Based on this quantification and other types of information, the system (or users of the system) may better assess the types and amount of resources needed to work towards resolving or satisfying the unmet technological need. While healthcare examples are provided herein by way of example, the present disclosure is not limited to any particular industry or application. For example, a financial institution may want to understand financial needs around a particular interest or location or an education system want to better understand which knowledge or skills is missing and in which communities. The disclosed embodiments may be used to quantify these needs based on data scraped from multiple sources. In some embodiments, the application may be local. For example, a corporation may scrape their internal data structures to seek unmet needs within the organization. Accordingly, while various examples are described below for context, the present disclosure are not limited to any particular application.

Some disclosed embodiments may be implemented to identify unmet technological needs. In some embodiments, this may include maintaining a data structure containing information about a plurality of unmet technological needs. As described above, a data structure may include any collection of information stored in a structured format, such as an array, a table, or various other formats. The data structure may be maintained through storage of the information in a storage location, updating information in the data structure, and/or by establishing or using a link for accessing information in the data structure. Such a data structure may include information about one or more unmet technological needs. As described above, an unmet technological need may include any circumstance or set of circumstances that require some course of action that is technical in nature, or could require a specific outcome. Some example unmet technological needs may include the need of diabetes patients for a treatment for a specific condition, the need for a previously undeveloped monitoring or diagnostic device to discover a specific condition within pregnant women, the need to treat a condition related to a new pandemic spreading across a general population, a specific population (e.g., elderly people, people in a particular geographic region, people with preexisting conditions, etc.), the need to treat a new vaccination side effect represented by specific symptoms (e.g., red skin), or various other needs. In some embodiments, an unmet technological need may be a more specific technical or procedural need, as indicated above. For example, for clinical trials may require transportation of a patient from a first location to a second location within a specific type of population. More specifically, members of underserved populations having a specific condition in a specific region may not own a car and thus may need help reaching a location that is greater than 0.7 miles or more from their home. The disclosed embodiments may capture a wide variety of unmet needs and may not necessarily be related to healthcare, as described above

The information associated with an unmet technological need may include any form of description, characteristic, classification, value, or other property of the unmet technological need. For example, this information may include an identifier of the unmet technological need (e.g., an assigned identifier, a unique identifier, a semi-unique identifier, a random or semi-random identifier, etc.) and a description of the unmet technological need. In some embodiments, the information may include a classification of the unmet technological need. A classification can include any specification of a class or subclass of an unmet technological need. For example, a classification may be specified based on an industry associated with the unmet technological need, a demographic associated with the unmet technological need, a type of the unmet technological need (e.g., a product need, a service need, a transportation-based need), or any other form of classification. As another example, the information may include a location associated with an unmet technological need, which may refer to a location where the unmet technological need is experienced, where a solution for the unmet technological need may be located, or various other locations. According to some embodiments, a location may be a virtual location, such as a place in a metaverse environment, or various other virtual environments. In some embodiments, the information may include an indication of an entity associated with the unmet technological need. This may include entities experiencing the unmet technological need, entities having skills associated with resolving the unmet technological need, entities having resources for resolving the unmet technological need, or any entity that may be otherwise associated with the unmet technological need.

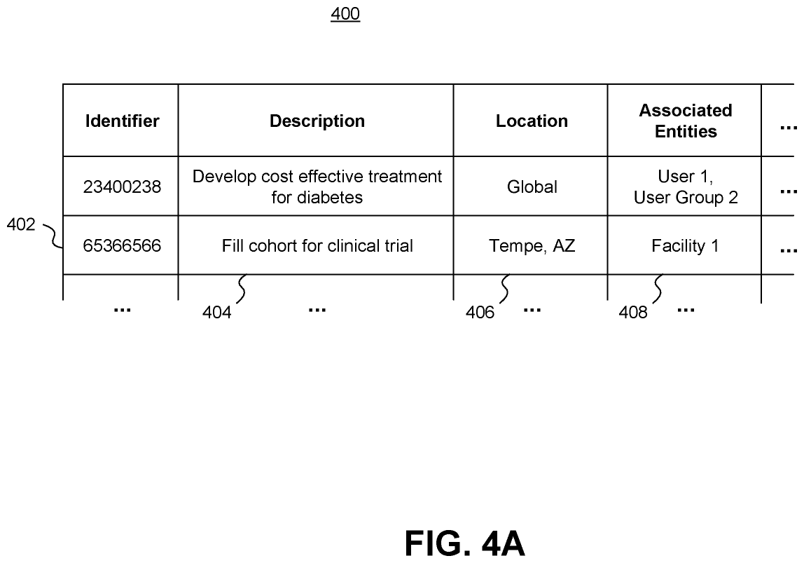

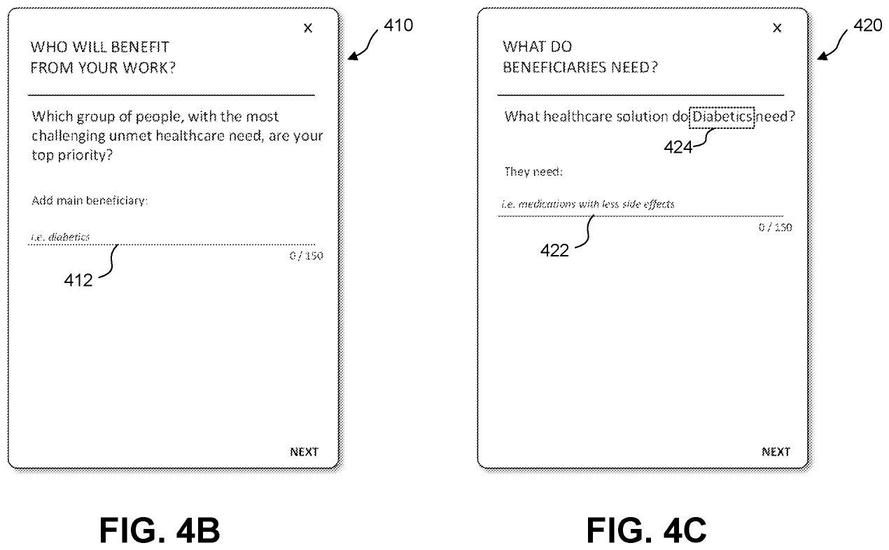

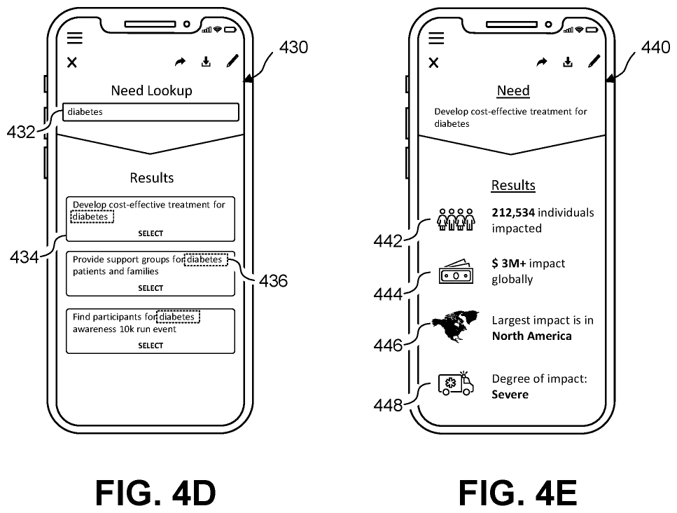

FIG. 4A illustrates example data structure 400 for storing information associated with an unmet technological need, consistent with the disclosed embodiments. In the example shown in FIG. 4A, unmet technological needs may be stored in a tabular format, with a plurality of rows indicating individual unmet technological needs and a plurality of columns indicating different information or properties of the unmet technological needs. For example, a particular unmet technological need may include a description 404 and may be assigned an identifier 402. Description 404 may be acquired in various ways. In some embodiments, the description may be input by a user, for example through one or more user interfaces 410 and/or 420 described below. Alternatively or additionally, the description may be generated by system 100. For example, the description may be extracted through scraping the internet for unmet technological needs and generating a classification of an unmet technological need based on the scraped information. In some embodiments, the description may be generated using a trained machine learning model. For example, a set of training data may be input into a machine learning algorithm, such as a neural network (e.g., an artificial neural network, a deep neural network, etc.) along with training output descriptions. The training data may include examples snippets of text, publications, user inputs, data structure, etc. along with corresponding descriptions of a corresponding unmet technological need. Accordingly, the model may be trained to generate descriptions for identified unmet technological needs based on other information scraped from the internet. While a neural network is provided by way of example, various other machine learning algorithms may be used as described above, including a logistic regression, a linear regression, a regression, a random forest, a K-Nearest Neighbor (KNN) model (for example as described above), a K-Means model, a decision tree, a cox proportional hazards regression model, a Naïve Bayes model, a Support Vector Machines (SVM) model, a gradient boosting algorithm, or any other form of machine learning model or algorithm. Alternatively or additionally the system may use a pattern recognition technique such as statistical technique to discover, classify, or extract unmet technological need from scraped resources. For example, a pattern recognition technique may indicate that the past unmet need may be found immediately after the word “require” in 80% of the examples. Accordingly, the inclusion of the word “require” in subsequent texts analysis by indicate that the following text includes an unmet technological need.

As shown in FIG. 4A, data structure 400 may include additional information, such as a location 406, an indication of associated entities 408, or various other forms of information. In some embodiments, the various forms of information described above may be split into subcategories. For example, the data structure may store indications of entities experiencing an unmet technological need separately from entities having a skill for resolving an unmet technological need. Accordingly, entities 408 represented in data structure 400 may be split into multiple columns. It is to be understood that the various forms of information shown in FIG. 4A and described above are provided by way of example, and that data structure 400 may include any form of information associated with an unmet technological need, including but not limited to any of the various types of information described throughout the present disclosure. In some embodiments, data structure 400 may be stored in a database, such as database 112 described above.

The information associated with the unmet technological needs that is maintained in the data structure may be collected in various ways. According to some embodiments, some or all of the information may be received through an input from a user or other entity. For example, this may include receiving inputs through user interface 310 and/or 320 via computing device 120, as described above. Accordingly, computing device 120 may receive a user input, generate a signal based on the user input and transmit the signal to a server (e.g., 110) over a network (e.g., network 140). The server may then parse the signal to extract information indicating the user input. In some embodiments, this may include encrypting or otherwise encoding the signal prior to transmission. FIGS. 4B and 4C illustrate additional example user interfaces for obtaining information about an unmet technological need, consistent with the disclosed embodiments. For example, user interface 410 may be presented to a user to identify types of entities that may benefit from a service or skill possessed by a user. For example, user interface 410 may be presented as part of an onboarding process in which an entity enters information to be represented in a platform or system. As shown in FIG. 4B, interface 410 may include an input field 412, through which a user may input a description of a type of beneficiary that may benefit from a skill of an entity. In some embodiments, the system may perform various semantic analysis or natural language processing techniques to classify or characterize the information input into field 412. For example, this may include a natural language processing algorithm to combine “diabetics,” “insulin users,” or other similar descriptors into the same classification. Accordingly, this may allow users to input descriptions of an unmet technological need using natural language to describe the unmet technological need. The system may then classify the descriptions to correlate or group similar needs so that they may be addressed in a similar fashion. Therefore, the system may address the same (or similar) unmet technological needs in the same manner, regardless of the format in which information is input by a user.

Similarly, user interface 420 may include a field 422 through which a user may input a description of an unmet technological need associated with a particular beneficiary or type of beneficiary. In some embodiments, the beneficiary may refer to the beneficiary identified through user interface 410. For example, user interface 420 may include an element 424 referencing data input through field 412. Element 424 may be an exact representation of the data entered in field 412, or may be a tokenized form (e.g., removing extraneous characters, changing characters from uppercase to lowercase), a classification of the input as described above, or various other representations. Through field 422, a user may input a description of the unmet technological need, which may be used to populate data structure 400. In some embodiments, a description may be stored exactly as it is input into field 422. Alternatively or additionally, a semantic analysis or natural language processing algorithm may be applied to the input to extract information. For example, this may include identifying keywords or phrases from within the input (e.g., “medication,” “cost,” “diseases,” “condition,”, “side effects”, “symptoms,” etc.) to determine a classification for an unmet technological need. Accordingly, although different users may describe an unmet technological need using different language, the system may classify the different descriptions into the same unmet technological need. In some embodiments, various other information, such as locations or entities may be extracted from the inputs into fields 412 and 422. Further, while fields 412 and 422 are shown by way of example, various other user input elements may be used. For example, user interfaces 410 and 420 may include drop-down lists or other elements, through which a user may select from a list of known beneficiaries or unmet technological needs (e.g., extracted from database 400).

According to some embodiments, the information associated with the unmet technological need may be obtained through scraping the internet. For example, server 110 (or another component of system 100) may access sources 150 through network 140. Server 110 may be configured to extract data representing unmet technological needs from sources 150, which may include social networking platforms, publication databases (e.g., including scholarly publications, professional publications, legal publications, etc.), news articles, medical records or databases, or various other sources accessible through network 140, including those described throughout the present disclosure. This may include extracting data from the scraped sources and performing a semantic analysis on the extracted data to identify unmet technological needs. This may also include cases where the source was determined by semantic analysis (e.g., sources that contain data entities that may be found in unmet technological needs as described above). For example, this may include scraping pages with historical use cases and finding phrases that match unmet need characteristics. For example, this may include analyzing a plurality of user inputs of unmet needs and using pattern recognition techniques to define combinations of elements that have a high likelihood (e.g., based on a score, etc.) of defining an unmet technological need. In some embodiments, this may further include performing an optical character recognition (OCR) technique to convert handwritten or printed text into machine-encoded text. While internet scraping is provided by way of example, the scraping may equally occur on local data sources, such as local database publications, local user profiles, or any other form of information. In some embodiments, the information associated with an unmet technological need may be aggregated from multiple sources. For example, data structure 400 may be constructed from data scraped from the Internet as well as data input through a user interface. This aggregation may include combining, merging, or summarizing data from different sources into the same unmet technological need, splitting data representing a single unmet technological need into representations of multiple unmet technological needs, or various other forms of data processing and/or manipulation.

Some disclosed embodiments may involve receiving a query, which may be associated with an unmet technological need. The query may be any event triggering identification of one or more unmet technological needs within the data structure. In some embodiments, the query may be initiated by a user. A query may be received in any electronic manner, such as in the form of signals transmitted over a network. For example, receiving the query may include receiving an input from a user through a user interface via computing device 120, which may generate a signal indicating the user input and transmit the signal to server 110. Server 110 may then parse the received signal to receive an indication of the input. In some embodiments, receiving the query may include receiving a text-based input, for example through field 422 or a similar field. For example, the text-based input may be a search term or phrase, a selected keyword, or the like. This may allow a user to search for unmet technological needs associated with a particular location, a particular industry, a particular type of need (e.g., filling a cohort, tracking research grant funding, etc.), or various other parameters. FIG. 4D illustrates an example user interface 430 through which a user may submit a query consistent with the disclosed embodiments. In this example, user interface 430 may include a text field 432 which may allow a user to enter one or more search terms, as shown in FIG. 4D. Another example may include a voice- or video-based input, which may include a search term or phrase, a selected keyword, or the like.

Alternatively or additionally, receiving the query may include receiving a selection of one or more properties of an unmet technological need, such as a category, a location, a beneficiary, an industry, a type, or various other information associated with an unmet technological need. For example, a user interface may present a list of topics, locations, or other properties from which a user may select one or more specific properties to include in the query. As another example, the query may include a document or multiple documents uploaded to server 110 or otherwise accessed by server 110, which may include information associated with one or more unmet technological needs that may be extracted by server 110, for example, using a semantic analysis or other natural language processing technique. In some embodiments, the query may be a trigger occurring separate from a user. For example, this may include a query that occurs periodically, a query based on an external event (e.g., publication of an article associated with an unmet technological need, onboarding of a user or entity, etc.), or various other triggers.

Some disclosed embodiments may further involve identifying in the data structure a subset of the plurality of unmet technological needs associated with the query. For example, this may include comparing information included in or associated with the query to information stored in the data structure. This may include performing a lookup function within particular fields of the data structure. Alternatively or additionally, this may include performing a search of an entire data structure. In some embodiments, identifying in the data structure a subset of the plurality of unmet technological needs associated with the query includes performing a semantic analysis on the query. For example, this may include extracting one or more keywords or phrases from a user input, document, or other body of text, which may represent characteristics of an unmet technological need to be compared to information in the data structure.

The subset of the plurality of unmet technological needs may represent one or more of the plurality of unmet technological needs determined to satisfy the query. In some embodiments, this may include scoring the unmet technological needs represented in the data structure based on a degree of matching to the query. Such scoring may be carried out via any appropriate algorithm, for example, regular expression (regex), conditional rules, and/or a fuzzy matching algorithm such as a trigram search. Alternatively or additionally, a gradient boosting machine (GBM) may be employed to score possible candidates for characteristics present in the query. In some embodiments, the subset of the plurality of unmet technological needs may be selected based on comparing matching scores associated with the unmet technological needs to a predetermined threshold, which may define a required degree of matching. Alternatively or additionally, this may include selecting a number of highest matches (e.g., the top 5, the top 10%, etc.) or various other methods for selecting a subset based on the matching score.

Some disclosed embodiments may further involve receiving a selection of a particular unmet technological need from the subset of the plurality of unmet technological needs. In some embodiments, receiving the selection of a particular unmet technological need may include displaying the subset of the plurality of unmet technological needs to a user, for example, through computing device 120. Referring to FIG. 4D, this may include displaying representations of the subset of the plurality of unmet technological needs in user interface 430 (or a separate user interface). For example, this may include displaying a representation of a particular unmet technological need 434, which a user may select through user interface 430 (e.g., by tapping the representation of particular unmet technological need 434, etc.). In some embodiments, the representations of the subset of the plurality of unmet technological needs may include other information about the unmet technological need, such as associated locations, entities, categories, or any of the various information described above. In some embodiments, user interface 430 may display an indication of why each unmet technological need is included in the subset. For example, this may include a highlighting or other marking 436 indicating a term or phrase from within a description of the unmet technological need believed to correspond to the query. As another example, user interface 430 may include a score for each unmet technological need indicating a degree of matching with the query, as described above.

Some disclosed embodiments may further involve receiving a request to identify an extent of the particular unmet technological need. The extent of an unmet technological need may include reference to a measurement, estimate, or other metric indicating the relative size or severity of an unmet technological need. The request may be received as signals over a network. For example, the request may be received at a server based on a transmission from a remote computing device. The extent of the particular unmet need may be defined in terms of a number of individuals or entities impacted by the unmet technological need, a percentage of a population impacted by an unmet technological need, a degree to which individuals or entities are affected by the unmet technological need, a severity or an amount of harm or impact cause by the unmet technological need, a monetary impact of the unmet technological need, an estimated monetary value needed to address the unmet technological need, or any other value or metric that may characterize how far an unmet technological need extends. The extent of the particular unmet need may thereafter be determined or estimated based on a scraping of the internet and an electronic analysis of the scraped information, as described previously and as elaborated on below.

In some embodiments, receiving the request to identify an extent of the particular unmet technological need may include a separate action by a user. For example, this may include selecting an “measure it” button or other element on user interface 430 (not shown). In some embodiments, the request to identify an extent of the particular unmet technological need may be initiated based on the selection of the particular unmet technological need. In other words, the identifying an extent of the particular unmet technological need may occur automatically in response to selection of the particular unmet technological need by the user, as described above. In some embodiments, the user may add more information about the unmet need, for example, through a free text entry, during a discussion, an interaction with other users/devices, or the like. In some embodiments, the selection of the particular unmet technological need and the request to identify the extent of the particular unmet technological need may occur during different timeframes (e.g., separated by minutes, hours, days, weeks, years, etc.). For example, a user may select the particular unmet technological need and server 110 may associate the particular unmet technological need with a user, which may include storing the particular unmet technological need in an associated manner with the user in a database or data structure. Then, at a later date or time, the user may submit a request to identify an extent of any particular unmet technological needs associated with the user. As another example, the request may be submitted automatically, for example, every week, every month, or based on other periodic or trigger-based events.

Based on the request, some disclosed embodiments may include scraping the internet to identify a plurality of sources containing information relating to the particular unmet technological need. As described herein, scraping the internet may include crawling across websites or other sources (e.g., sources 150 described above) and extracting data from the sources. For example, this may include accessing webpages, social media platforms, publications, news articles, databases, or any other sources that may include information associated with an unmet technological need. In the example of diabetes treatments, this may include identifying and scraping a social media platform for people mentioning related terms, such as “diabetes,” “blood glucose,” “insulin”, “ulcers,” or other indications that the user may be suffering from diabetes. As another example, an unmet technological need may include repairing a defect experienced when using a product (e.g., sugar level monitor device, wound tracker, etc.). Accordingly, the system may crawl websites searching for mentions of the particular defect, symptoms of the defect, or other indications that a user is experiencing the defect. For example, patients may describe particular symptoms of condition that may be connected to a specific disease (e.g., frequent urination, urinating at night, unintended weight loss, or other symptoms, which may be connected to diabetes or other conditions) in question and answer sites, patient forums, or various other discussion forums.

Some disclosed embodiments may further involve performing semantic analysis on the plurality of scraped sources to determine, in each scraped source, information characterizing an extent of the particular unmet technological need. The information characterizing the extent of the particular unmet technological need may include any form of measurement, estimation, value, description, rating, or other information that would characterize the extent of the need. In some embodiments, the information characterizing the extent of the particular unmet technological need may include a number of individuals or entities (which may also be referred to as beneficiaries) experiencing the particular unmet technological need. As another example, the information characterizing the extent of the particular unmet technological need may include an indication of how close an industry is to resolving the unmet technological need. For example, the semantic analysis may identify words or phrases such as “FDA approval,” “clinical trials,” “promising drug compound” or other terms that may indicate how far along a treatment is in a development process. Other terms may similarly be used in other applications, such as with product development, software development, or the like. As another example the information about the characterizing the extent of the particular unmet technological need may include a number of solutions, specialists, or solution providers that claim to have solutions for resolving or addressing the unmet need.

As another example, the information characterizing the extent of the particular unmet technological need may include an indication of a severity of the impact of the particular unmet technological need. For example, the sematic analysis may identify terms such as “severe,” “fatal,” “devastating,” or any other suitable terms that may indicate a degree to which the particular unmet technological need is impacting an individual or entity. The severity may be defined in terms of monetary impact, health impacts (e.g., mortality rate, etc.), a duration the need has been experienced, a number or estimated number of individuals impacted, physical damage caused, a degree of disruption to normal activities, or various other metrics, which one skilled in the art would recognize depending on the particular industry or application. In yet another example, the information characterizing the extent of the particular unmet technological need may include an indication of a location or region associated with the impact. For example, the system may extract, through the semantic analysis, indications of associations or regions where the impact is being experienced or regions where the impact is more significant as compared to other regions.

Some disclosed embodiments may further involve aggregating the characterization information from each scraped source. In this context, the aggregation may include any form of summation, summarization, averaging (e.g., mean, weighted average, running average, etc.), standard deviations or other statistical values, categorization, classification, or any other forms of combined analysis. For example, if the characterization information includes an estimated economic impact of the particular unmet technological need from multiple distinct sources, the aggregation may include generating a total economic impact (or a total estimated economic impact). As another example, if the characterization information includes an estimated degree of impact of the particular unmet technological need, the aggregation may include determining an average impact of the particular unmet technological need. In some embodiments, the aggregation may be performed relative to different subcategories of beneficiaries. For example, the aggregation may include generating total impacts for particular regions, social classes, ethnicities, genders, date ranges, equipment or product types (e.g., MRI machine, eye scanner, insulin pumps, etc.) or various other subcategories.

Some disclosed embodiments may further involve analyzing the aggregated characterization information to quantify a number of beneficiaries sharing the particular unmet technological need. In some embodiments, the analysis may be performed in conjunction with the aggregation and may not necessarily be a separate analysis. For example, where the aggregation includes a summation technique, the analysis may be accomplished through the summation process, such that the resulting sum may represent the quantity of the number of beneficiaries. This may be true for other forms of aggregation and analysis as well. In some embodiments, the analysis may be a separate step performed on the aggregated characterization information. For example, this may include a separate algorithm, function, series of functions, comparison, verification, statistical analysis, or other form of analysis to determine the quantification.

The beneficiaries may include any individual or other entity that shares the particular unmet technological need. In some embodiments, the beneficiaries may include individuals sharing the particular unmet technological need. For example, the individuals may include patients suffering from a medical condition associated with the particular unmet technological need. Continuing with the example above, the patients may be suffering from diabetes and may be in need of treatment or more cost-effective treatment. As another example, the beneficiaries may include communities sharing the particular unmet technological need. For example, the communities may include a geographic region affected by the particular unmet technological need. The geographic region may be defined in various ways. For example, the geographic region may be specified through an input of a user. Alternatively or additionally, the geographic region may be identified based on data accessible by system 100. For example, computing device 120 may be a mobile device or another device including a global positioning system (GPS) sensor or another sensor that may be used to determine a location of user 130. Server 110 may parse data packets received from computing device 120 to ascertain a geographic location of user 130.

According to some embodiments, various other forms of quantifications associated with the unmet technological need may be determined based on the aggregated characterization information. For example, the disclosed methods may include analyzing the aggregated characterization information to quantify an economic impact associated with the unmet technological need. As indicated above, the economic impact may include any monetary-based effect of a particular unmet technological need. For example, if the cost of a particular treatment is known and the number of beneficiaries is determined as described above, the economic impact may be represented as the cost of the treatment multiplied by the number of beneficiaries (or estimated number of beneficiaries). In some embodiments, the economic impact may be defined in terms of lost resources. For example, depending on the particular unmet technological need, the unmet technological need may result in a loss of work-hours, school-hours, workforce, plant or facility operation, energy production, or the like, which may have adverse economic effects. As another example, the particular unmet technological need may require additional hospitalizations, attention of rescue or emergency workers, or the like, which similarly may be evaluated in economic terms. In some embodiments, the economic impact may be limited to a particular category or other subgroup of beneficiaries. For example, the economic impact may include an economic impact on particular communities sharing the particular unmet technological need. As another example, the economic impact may include an economic impact on an industry associated with the particular unmet technological need.

As another example, some disclosed embodiments may involve analyzing the aggregated characterization information to quantify a degree of impact of the particular unmet technological need. A used herein, a degree of impact for an unmet technological need may refer to a level of impact the unmet technological need has on those affected by the unmet technological need. This degree of impact may be useful, for example, to identify or prioritize unmet technological needs that need to be addressed. For example, in the context of a healthcare-related unmet technological need, the system may aggregate data from multiple sources to determine a number of patients dying from the unmet technological need, or various other indicators of the severity of the unmet technological need. The degree of impact may be expressed in various formats, such as a percentage, on a numerical scale (e.g., 1 to 10, 1 to 5, 1 to 100, etc.), in a text-based rating or classification (e.g., “minor,” “severe,” etc.), or various other formats that may indicate a relative degree of impact. In embodiments where the beneficiaries are communities sharing the particular unmet technological need, this may include quantifying a degree of impact on the communities sharing the particular unmet technological need. For example, the unmet technological need may be an unmet healthcare need and the degree of impact may be associated with a quality of life of individuals in the communities. As used herein, a quality of life may refer to the standard of health, comfort, and happiness experienced by an individual or group. In some embodiments, the quality of life may be represented according to a predefined quality of life index (e.g., Quality Adjusted Life Year (QALY), on a numerical scale, etc.). The quality of life may take into consideration a variety of factors including health, living conditions, financial stability, or various other factors that may impact a health or happiness of an individual. In embodiments where the beneficiaries are individuals sharing the particular unmet technological need, this may include quantifying a degree of impact on the individuals sharing the particular unmet technological need. For example, the unmet technological need may be an unmet healthcare need and the degree of impact may be associated with a quality of life of the individuals.

Some disclosed embodiments may further involve outputting for presentation the quantification in association with an indication of the particular unmet technological need. Outputting the quantification for presentation is not limited to any particular form of output and may include any process in which data is made available such that it may be presented, either immediately or at a later time. In some embodiments, the outputting may include transmitting the quantification and an indication of the particular unmet technological need to another device, such as computing device 120. As another example, outputting the quantification for presentation may include storing the quantification in association with the indication of the particular unmet technological need. For example, this may include storing the quantification of the number of beneficiaries in data structure 400 (e.g., in an additional column) or in a separate data structure. Accordingly, the quantification may be made accessible to a device in a manner allowing the device to present the quantification as needed.

In some embodiments, outputting the quantification for presentation may include causing the quantification to be displayed on a user interface, for example, through display 272. FIG. 4E illustrates an example user interface 440 for displaying a quantification of a number of beneficiaries sharing the particular unmet technological need and various other quantifications associated with an unmet technological need, consistent with the disclosed embodiments. For example, user interface 440 may be displayed based on a selection of unmet technological need 434, as described above. As shown in FIG. 4E, user interface 440 may include an element 442, which may indicate a number of beneficiaries sharing the particular unmet technological need. User interface may also include an element 444 indicating an economic impact associated with the unmet technological need, an element 446 indicating a geographic region associated with the unmet technological need, and an element 448 indicating a degree of impact associated with the unmet technological need. While elements 442, 444, 446, and 448 are provided by way of example, various other forms of information or manners of representing the information may be used. For example, user interface 440 may include various other forms of icons, graphics, text elements, graphs, charts, tables, animations, or the like.

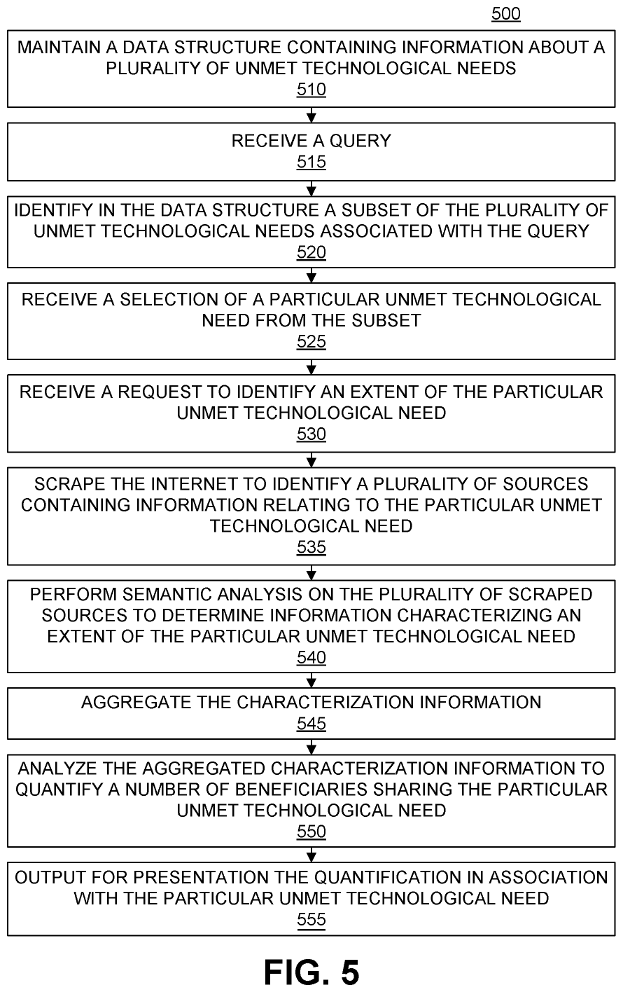

FIG. 5 is a flowchart showing an example process 500 for identifying unmet technological needs, consistent with the disclosed embodiments. Process 500 may be performed by at least one processor, such as processor 210. It is to be understood that throughout the present disclosure, the term “processor” is used as a shorthand for “at least one processor.” In other words, a processor may include one or more structures that perform logic operations whether such structures are collocated, connected, or dispersed. In some embodiments, a non-transitory computer readable medium may contain instructions that when executed by a processor cause the processor to perform process 500. Further, process 500 is not necessarily limited to the steps shown in FIG. 5, and any steps or processes of the various embodiments described throughout the present disclosure may also be included in process 500, including those described above with respect to FIGS. 4A, 4B, 4C, D, and 4E.

In step 510, process 500 may include maintaining a data structure containing information about a plurality of unmet technological needs. For example, this may include maintaining data structure 400, as described above.

In step 515, process 500 may include receiving a query. In some embodiments, receiving the query may include receiving an input from a user through a user interface. For example, this may include receiving an input into text field 432, as described above. In step 520, process 500 may include identifying in the data structure a subset of the plurality of unmet technological needs associated with the query. In some embodiments, this may include performing a semantic analysis on the query, as described above. For example, this may include performing a semantic analysis to determine a keyword or category of unmet technological needs and identifying a subset of the plurality of unmet technological needs associated with the keyword or category.

In step 525, process 500 may include receiving a selection of a particular unmet technological need from the subset of the plurality of unmet technological needs. For example, step 525 may include causing representations of the subset of the plurality of unmet technological needs to be displayed on a user interface (e.g., user interface 430) and receiving the selection of a particular unmet technological need may include receiving a selection of one of the representations of the subset of the plurality of unmet technological needs (e.g., unmet technological need 434) by a user. In some embodiments, step 525 may be combined with or may occur instead of one or more of the previous steps. For example, in some embodiments, rather than receiving a separate query and identifying a subset of unmet technological needs, process 500 may include receiving a selection of the particular unmet technological need from the plurality of unmet technological needs maintained in the data structure.

In step 530, process 500 may include receiving a request to identify an extent of the particular unmet technological need. In some embodiments, this may be based on the selection of the unmet technological need in step 525. In other words, by selecting the particular unmet technological need, a user may indicate they wish to identify the extent of the particular unmet technological need. Alternatively or additionally, the request to identify an extent of the particular unmet technological need may be a separate action by a user or may be based on another event (e.g., a predetermined time interval, etc.).

In step 535, process 500 may include scraping the internet to identify a plurality of sources containing information relating to the particular unmet technological need. For example, this may include scraping the internet to identify one or more of sources 150, as described above. In step 540, process 500 may include performing semantic analysis on the plurality of scraped sources to determine in each scraped source information characterizing an extent of the particular unmet technological need. In some embodiments, the information characterizing an extent of the particular unmet technological need may include an indication of how close an industry is to resolving the unmet technological need, as described above. In step 545, process 500 may include aggregating the characterization information from each scraped source. For example, this may include summarizing, combining, comparing, differentiating, averaging, or any other form of aggregation of data.

In step 550, process 500 may include analyzing the aggregated characterization information to quantify a number of beneficiaries sharing the particular unmet technological need. As descried above, the beneficiaries may refer to various types of entities. In some embodiments, the beneficiaries may include communities sharing the particular unmet technological need, as described above. For example, the communities may include a geographic region affected by the particular unmet technological need. In this example, step 550 may further include analyzing the aggregated characterization information to quantify a degree of impact on the communities sharing the particular unmet technological need. For example, the unmet technological need may be an unmet healthcare need and the degree of impact is associated with a quality of life of individuals in the communities. In some embodiments, the beneficiaries may include individuals sharing the particular unmet technological need. For example, the individuals may include patients suffering from a medical condition associated with the particular unmet technological need. In this example, step 550 may further include analyzing the aggregated characterization information to quantify a degree of impact on the individuals sharing the particular unmet technological need. For example, the particular unmet technological need may be an unmet healthcare need and the degree of impact is associated with a quality of life of the individuals.

In some embodiments, various other quantifications associated with the particular unmet technological need may be determined. For example, process 500 may further include analyzing the aggregated characterization information to quantify an economic impact associated with the unmet technological need, as described above. For example, the economic impact may include an economic impact on communities sharing the particular unmet technological need, an economic impact on an industry associated with the particular unmet technological need, or various other forms of economic impacts. As another example, process 500 may further include analyzing the aggregated characterization information to quantify a degree of impact associated with the unmet technological need. This may include a severity of the particular unmet technological need or various other metrics that may indicate a degree of impact.

As another example, process 500 may further include analyzing the aggregated characterization information to quantify an implementation status of the unmet technological need. The implementation status may include any indicator of how close the unmet technological need is to being resolved. For example, the implementation status may include an indication of how many solutions to serve or resolve the particular unmet technological need exist. As another example, the implementation status may include an indication of how many entities or experts are working towards resolving the unmet technological need, how many resources are targeted to solve the unmet technological need, or the like. This information may be accessed from the scraped sources, similar to the various other forms of aggregated information described herein. For example, this may include scraping the internet to identify scholarly articles associated with an unmet technological need and determining a total number of unique researchers working towards resolving the unmet technological need. This indication of an implementation status may help users to identify or prioritize unmet technological needs that may be underserved to determine where to focus various resources.

In step 555, outputting for presentation the quantification in association with an indication of the particular unmet technological need. In some embodiments, this may include causing the quantification to be displayed on a device such as computing device 120. For example, step 555 may include causing user interface 440 or a similar user interface to be displayed on computing device 120.

As noted above, embodiments disclosed herein may be applied to a wide range of unmet technological needs. An exemplary use case consistent with some embodiments is provided below. It is understood that this use case is provided by way of example and is not limiting of the present disclosure in any way. As one particular example, an organization may be looking to invest millions of dollars to address a growing substance abuse problem within a particular community. Embodiments disclosed herein may allow the organization to quantify various aspects of this issue to determine how and where their investment would provide the greatest impact. Accordingly, a user representing the organization may submit a query related to an unmet technological need (step 515). For example, a user may input the phrase “assistance with substance abuse” into a user interface, such as text field 432 of user interface 430. This query input (or a signal representing the input) may be transmitted to server 110.

Server 110 may maintain a database of unmet technological needs (step 510) and may identify a subset of unmet technological needs relating to the query (520). In this example, the server may identify a keyword of “substance abuse” and may compare this keyword to information characterizing the unmet technological needs. This may include performing a look-up function in data structure 400 to identify two or more unmet technological needs associated with substance abuse. For example, server 110 may identify a subset of unmet technological need including “treatment of growing substance abuse issues in inner-cities” and “identify cohort of patients for clinical trial of new substance abuse prevention drug.” The user may then select the most relevant unmet technological need (in this case, the first example) through user interface 430 (step 525). In some embodiments, server 110 may recognize this selection as a request to identify an extent of the particular unmet technological need (step 535). Alternatively, the user may select a specific element in a user interface to initiate the identification of the extent of the particular unmet technological need.

Sever 110 may then scrape various Internet sources containing information related to treatment of substance abuse (step 535). In this example, server 110 may extract information from news articles discussing the substance abuse issues in various communities. Server 110 may scrape various other sources, such as a Facebook™ or other social medial platforms, to identify posts that may pertain to substance abuse. In some embodiments, the system may expand the scope of the search to include various similar keywords. For example, the system may maintain a keyword index identifying terms such as “alcoholic,” “heroin,” “relapse,” or various terms as having potential relevance to the term “substance abuse.” A semantic analysis may be performed on this information to glean information characterizing the extent of the issue (step 540). For example, various news articles may describe an approximate size of a population that is affected by substance abuse issues in different regions. The number of Facebook™ posts and geographic locations associated with the posts may provide additional context.

This information may then be aggregated (step 545) and then analyzed (step 550) to quantify a size of the population that is affected by substance abuse issues. In some embodiments, the analysis may identify specific cities that are impacted the most, and may further identify specific classes of individuals that are impacted the most. As another example, the information may indicate the severity of the substance abuse problem. For example, articles including keywords such as “overdose,” “fatalities,” or other keywords may indicate the relative severity of the issues among different communities. This information may then be provided to the user (step 555) via user interface 440. Accordingly, the organization may determine which cities and classes of individuals would benefit most from their financial contributions, allowing for a strategic deployment of their efforts and distributions. This use case is provided by way of example, and process 500 may be applied to numerous other unmet technological needs in a wide variety of industries.

As indicated herein, embodiments consistent with the present disclosure provide a significant improvement over existing systems. For example, a user may select a particular unmet technological need and receive up-to-date assessments of the extent and severity of the unmet technological need. In particular, information scraped from a wide variety of current sources, such as news articles, publications, social media platforms, or other sources, may be analyzed in response to a user query. This aggregation from multiple different sources provides a more robust and current assessment of the unmet technological need than would be available from any conventional systems. Accordingly, embodiments consistent with the present disclosure provide, among other advantages, improved accuracy, efficiency, convenience, and functionality over conventional techniques.

In some embodiments, process 500 may be implemented in combination with various other techniques described herein. For example, algorithms may be applied to the sources and information scraped from the internet, to predict an unmet need or multiple unmet needs and present them to a user or users even before the user has specified the unmet need or needs. Accordingly, a cluster of users with the same unmet need may be generated, and users within the cluster may be presented them with the same, or similar, solutions. When or if a user accepts or confirms the unmet need, the user may be presented with even more potentially related unmet needs, thereby giving the user solutions for future challenges and/or problems. Various other example implementations are described throughout the present disclosure.

As described throughout the present disclosure, the disclosed systems, methods and computer readable media may be implemented to identify an unmet technological need. Once a need has been identified, it may be desirable to determine requirements for fulfilling the need, and to identify a potential solution based on the determined requirements. In some embodiments, previous solutions meeting these requirements may provide insights into resolving a current unmet technological need. Some embodiments may include leveraging information pertaining to one or more previous solutions to determine if others were successful, and whether the solution is safe and economically feasible. For example, information about these previous solutions, as well as information indicating an associated efficacy, safety, cost-effectiveness, practicability, or any combination thereof may be accessed through scraping the Internet. If a feasible solution is identified, an output may be generated including a recommendation to adopt the solution. In some embodiments, multiple solutions may be identified and presented to a user.

Some disclosed embodiments may be implemented to receive an indication of an unmet technological need of at least one entity. As described above, an unmet technological need may include any circumstance or set of circumstances that require some course of action that is technical in nature. An indication of the unmet technological need may include any form of data or information that identifies a particular unmet technological need. For example, in some embodiments, various unmet technological needs may be represented in a data structure, such as data structure 400. Accordingly, the indication of the unmet technological need may be a description of an unmet technological need, which may correspond to or may be compared to description 404 stored in data structure 400. As another example, the indication of the unmet technological need may be an identifier, which may correspond to or may be compared to identifier 402 stored in data structure 400. Accordingly, the indication may include data that is used to perform a look-up function on a data structure storing information about a plurality of unmet technological needs.

Receiving the indication of an unmet technological need may occur in various ways. The receiving may occur through actively accessing the indication (e.g., through generating a query, accessing a data source, etc.) or through passively receiving the indication (e.g., through receiving a notification, a prompt, etc.). In some embodiments, the indication of the unmet technological need may be received through an input from a user. For example, a user interface may be presented through which a user may input or select an unmet technological need. As one example, interface 430 may be presented, which may include a text field 432 to allow a user to enter one or more search terms, as described above with respect to FIG. 4D. Interface 430 may then display representations of a subset of the plurality of unmet technological needs, such as a representation of a particular unmet technological need 434, which a user may select through user interface 430 as described above. In some embodiments, the indication may have been received previously. For example, an entity may indicate a particular unmet technological need during an onboarding process and the unmet technological need may be associated with a user in a data structure. Accordingly, the receipt of the indication of the unmet technological need may include identifying an unmet technological need that has previously been associated with the entity. Alternatively or additionally, a new unmet technological need may be defined, for example through interfaces 410 and 420. Any other manner through which an indication of particular unmet technological need may be selected, identified, specified, or produced may constitute receiving the indication of the unmet technological need.

Some disclosed embodiments may further involve electronically extracting data from a data source to identify a plurality of requirements for fulfilling the unmet technological need. As used herein, a requirement for fulfilling the unmet technological need may include any form of specification, constraint, or condition that should be met in order to satisfy the unmet technological need. In some embodiments, one or more of the requirements may be defined in terms of prerequisites for achieving a need. For example, the requirements may correspond to various steps, tasks, resources, or preparations that would be needed or desirable for fulfilling the unmet technological need. As one particular example, an unmet technological need may be a need to fulfil a clinical trial, as described throughout the present disclosure. Various requirements may include acquiring access to certain test facilities or laboratories, identifying a professional with certain clinical expertise, identifying a cohort of patients to participate in the trial, or similar requirements. As another example, an unmet technological need may be defined to open a new mental health department of a medical facility or medical practice group. Associated requirements may include identifying qualified personnel (e.g., physicians, receptionists, nurses, etc.), securing an office space, finding specific training, etc. Another example may include a need to procure a new magnetic resonance imaging (MRI) machine and requirements may be defined as obtaining funding to buy the machine, identifying a space for the machine, finding available training resources for use of the machine, or the like.

According to some embodiments, one or more of the requirements may be framed in terms of goals associated with fulfillment of the unmet technological need. In other words, the requirements may be conditions that, when met, may indicate that an unmet technological need was satisfied or a degree to which the unmet technological need was satisfied. For example, where an unmet technological need is defined as a need to fulfil a clinical trial, requirements may be extracted from trial protocol parameters. This may include a number or patients to be tested, a type or characteristic of patients having been tested, a procedure in which patients are tested or the like. According to some embodiments, one or more of the requirements may be framed in terms of constraints or rules associated with fulfillment of the unmet technological need. Accordingly, the requirements may impose a limitation or restriction on the types of solutions available to satisfy an unmet technological need. For example, a requirement may be that the solution be performed in a particular geographic location or in a virtual environment, using a specified number of individuals, using one or more predefined resources (e.g., must be performed using a specified facility, etc.), within a particular time or budget requirement, or the like.

The data for identifying the requirements may be electronically extracted from a data source in various ways. Electronically extracting data may include any form of computer-based process in which information is obtained. The extracting may include any form of querying the data source, accessing the data source, performing a search, performing a look-up function, parsing text or other data, or any other computer-based functions that may be used to obtain data. The data source may include any form of electronically accessible source of information. In some embodiments, the data source may be a database or other form of data structure, such as data structure 400. For example, data structure 400 may include one or more fields specifying requirements associated with a particular unmet technological need. Accordingly, extracting the requirements may include performing a look-up function within data structure 400 based on the indication of the unmet technological need. Alternatively or additionally, the requirements may be extracted from a different data structure, separate from data structure 400. The data source may include various other types of data sources such as web platforms, webpages, cloud-based storage platforms, local or network-based hard drives or other memory devices, or any other form of electronically accessible medium.

According to some embodiments, electronically extracting the data may include scraping from the Internet or other sources data from which the requirements may be derived. For example, this may include scraping various sources 150 to identify articles, case studies, plans, stories, or other forms of documents or information that may indicate various requirements for fulfilling an unmet technological need. Data from multiple differing sources and such forms of documents or information may be aggregated to derive the requirements. Scraping to aggregate information in this context and in the other contexts of scraping described herein enables needs and requirements to be identified to an extent that would be unachievable in any other way. In a world flooded with information, even with standard web searching tools like Google, finding sufficient relevant information at a level of granularity necessary to identify appropriate sources of information let alone specific cases within those sources is unreliable if not unachievable.

Continuing with the clinical trial example, scraping the Internet may be used to identify trial protocol parameters for similar trials (e.g., of a similar size, within the same geographic region, pertaining to the same disease or treatment, etc.) and parameters extracted from the similar trials (e.g., obtained using semantic analysis to understand context) may be proposed or set as requirements for a current trial. Alternatively or additionally, the system may scrape historical use cases, discussions, reports, books, and/or various other data sources about similar clinical trials. For example, this may include trials with the same indications, conditions, type of sites, or other common properties. Sematic analysis may then be used to recognize requirements that are connected to the unmet need. by comparing different sources, the system can generate a scoring to determine, for a given unmet need and combination of other parameters, what the relevant requirements are. For example, the requirements may be different if various parameters are different, even if the unmet need is the same. Accordingly, dynamic and current requirements lists may be built based on all available and published knowledge to run, based on past experience, any clinical trial dynamically at scale.

In some embodiments, one or more of the requirements may be extracted using a machine-trained model. For example, training data including data describing an unmet technological need (e.g., documents, snippets of text, etc.) along with corresponding sets of requirements may be input into a machine learning algorithm, such as a neural network (e.g., an artificial neural network, a deep neural network, etc.). Accordingly, the model may be trained to extract requirements based on other information describing an unmet technological need. Continuing with the clinical trial example, one or more documents describing a protocol or parameters for a trial (either as structured or unstructured text, or a combination thereof) may be input into a machine learning model along with specified requirements that are ascertainable from the documents. Accordingly, for future clinical trials similar documents may be input into the trained model to extract requirements. While a neural network is provided by way of example, various other machine learning algorithms may be used as described above, including a logistic regression, a linear regression, a regression, a random forest, a K-Nearest Neighbor (KNN) model (for example as described above), a K-Means model, a decision tree, a cox proportional hazards regression model, a Naïve Bayes model, a Support Vector Machines (SVM) model, a gradient boosting algorithm, or any other form of machine learning model or algorithm.

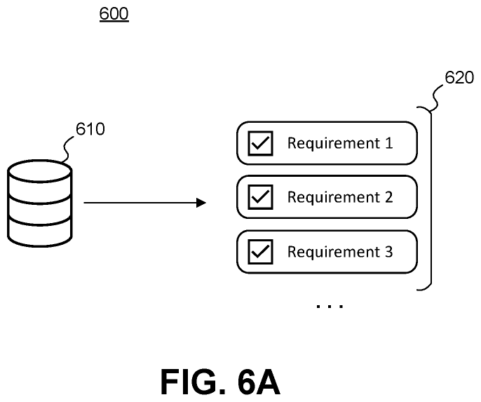

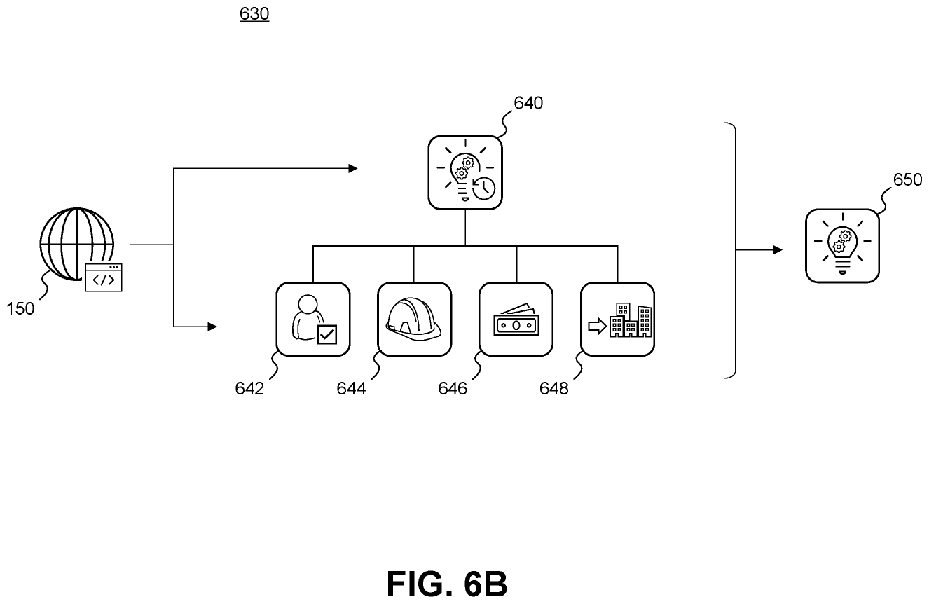

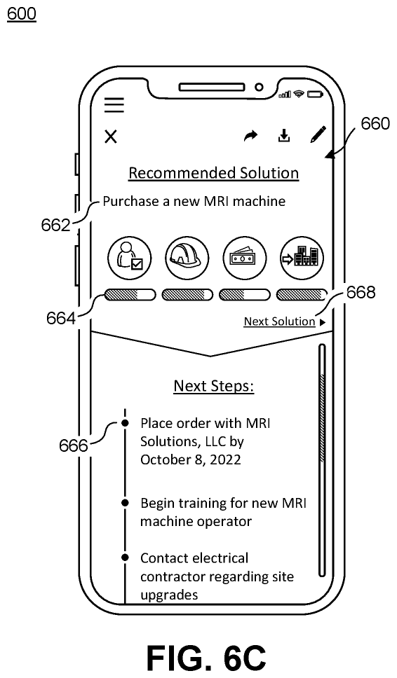

FIG. 6A is a block diagram illustrating an example process 600 for extracting data from a data source to identify a plurality of requirements, consistent with some embodiments of the present disclosure. For example, the data may be extracted from a data source 610 by server 110. In some embodiments, data source 610 may be a local data source and may correspond to database 112. Alternatively or additionally, data source 610 may be a remote data source accessed through network 140. As described in further detail above, data source 610 may include a database, a drive or other data storage device, a website or internet-based source, a cloud platform, or the like. More broadly, the data source may be the world wide web, or segments of the world wide web determined by at least one processor to be relevant to a particular case. Data extracted from data source 610 may be parsed, analyzed, searched, aggregated, or otherwise processed to extract one or more requirements 620, as shown in FIG. 6A.

Some disclosed embodiments may involve scraping the Internet to identify at least one solution satisfying at least one of the plurality of requirements. As used herein, a solution may be any form of means for solving a problem, and may include one or more actions taken by an entity or a result achieved through the one or more actions. In this context, a solution may refer to a previous action that was performed or a previous result that was achieved that satisfies at least one of the plurality of requirements. For example, a solution may include a procedure or process that was followed, an operation, a product that was developed, a service that was offered, software, a team that was developed, a resolution that was reached, or various other forms of solutions. As one skilled in the art would recognize, a solution may be dependent on the type of unmet technological need such as the technological field, a size or scope of the unmet technological need, an industry associated with the unmet technological need, or the like. Accordingly, although various example solutions are provided herein, a solution is not limited to any particular example and may include a wide variety of solutions or types of solutions.

As described throughout the present disclosure, scraping the internet may include the use of one or more web crawlers and extraction bots to acquire data from a plurality of sources. For example, server 110 (or another component of system 100) may access sources 150 through network 140. In this context, server 110 may be configured to extract data representing solutions satisfying at least one of the plurality of requirements from sources 150, which may include social networking platforms, publication databases (e.g., including scholarly publications, professional publications, legal publications, etc.), news articles, medical records or databases, webpages, product catalogs, source code servers, or various other sources accessible through network 140. For example, the at least one solution may be ascertained from a social network platform (e.g., LinkedIn™, Facebook™, etc.), publications related to the current unmet technological need, or various other Internet-based sources, websites containing descriptions of a product or a service, Q&A websites/products (e.g., Quora™, Stackoverflow™, etc.), professional directories, apps, associations, or organizations (diaglobal.org, acrpnet.org) professional on-line journals (science.org, nejm.org), online use cases, professional publications including scientific publications (e.g., WebMD™, ResearchGate™), government online sources (e.g., clinicaltrials.gov, fda.gov), online jobs or (e.g., Upwork™, Indeed™) eBooks, online Research service providers (e.g., CBinsight™, Gartner™, etc.) and/or any other suitable sources, In some embodiments, this scraping may include performing a semantic analysis on the scraped information, as described herein. For example, this may include processing natural language text to ascertain information about one or more solutions, how they were implemented, and whether they satisfy the at least one requirement. In some embodiments, this may further include performing an optical character recognition (OCR) technique to convert handwritten or printed text into machine-encoded text. For example, a requirement to measure leg ulcers by the patients at home may be identified, and the disclosed embodiments may include recognizing that a leg ulcer could be a symptom of diabetes. Accordingly, sources such as product websites, app stores, service websites, publications or the like that connect themselves as related to diabetes (by many ways such as titles, SEO, tags) may be scanned to extract content of the pages and also related materials that are connected (e.g., PDF of a product description, brochure, use cases and more). Sematic analysis may be performed to identify the requirement through phrases or similar patterns as described above. It is to be understood that in this example, diabetes is only one possible pathway and similar processes may be performed on all alternative pathways that are identified.

While internet scraping is provided by way of example, the scraping may equally occur on local data sources, such as local database publications, local user profiles, or any other form of information. In some embodiments, the information identifying a solution may be aggregated from multiple sources. For example, a solution may be identified through aggregation of information scraped from the Internet as well as data input through a user interface. This aggregation may include combining, merging, or summarizing data from different sources into the same unmet technological need, splitting data, or various other forms of data processing and/or manipulation.

A solution may be considered to satisfy at least one requirement based on some form of indication in the scraped data that the requirement is met through the solution. For example, a news article may indicate that a particular program that was implemented improved or solved a particular social issue (e.g., hunger, homelessness, etc.) within a particular community. As another example, a medical journal publication may indicate that a clinical trial was performed successfully and may provide details as to how the trial was implemented. In some embodiments, the determination of whether a particular solution satisfies a requirement may be a binary determination. In other words, a solution may be determined to either satisfy a particular requirement or not. Alternatively or additionally, a determination of whether a solution satisfies a requirement may be represented as a degree to which the solution satisfies the requirement. For example, if a requirement is defined to stay within a certain budget and an identified solution exceeds the budget slightly, the solution may be determined to have a 90% compliance with the requirement (or any other suitable measure of degree) based on how close the solution is to meeting the requirement. In some embodiments, the degree may be compared to a predetermined threshold to determine whether the solution is considered to satisfy the requirement or not. Alternatively or additionally, the determined degree may be used for further analysis (e.g., selecting a proposed solution from multiple possible solution, etc.). Some embodiments may further include a confidence level or rating associated with whether the solution meets a requirement. The confidence level may be an indication of a degree of certainty that a solution meets a requirement. For example, if a publication indicates the solution meets a requirement in passing without further discussion, the solution may be assigned a lower confidence rating as compared to a solution where multiple publications indicate in several instances that the solution meets the requirement. This confidence level may be distinct from a degree to which the solution meets the requirement. For example, a solution may be determined to meet a requirement in full, but may have a relatively low confidence rating, or vice versa.

In some embodiments, the at least one solution may include a plurality of solutions. The plurality of solutions may be alternative solutions satisfying one or more of the same requirements, may each satisfy different requirements, or any combination thereof. For example, scraping the internet to identify at least one solution satisfying at least one of the plurality of requirements may include identifying a first solution satisfying all of the plurality of requirements and a second solution satisfying all of the plurality of requirements. The first and second solutions may be compared based on a degree to which they each meet the at least one requirement, a confidence rating of each of the solutions, or various other scores as discussed in further detail below. As another example, a first solution may be determined to satisfy one of the plurality of requirements and a second solution may be determined to satisfy another of the plurality of requirements. Accordingly, a combination of both the first and second solutions may be recommended in order to meet all of the requirements. As another example, five requirements for fulfilling an unmet technological need may be identified and scraping the internet to identify at least one solution may include identifying a first solution meeting four of the requirements and a second solution satisfying three of the requirements, each solution satisfying each of the requirements to varying degrees. Accordingly, the plurality of solutions may each satisfy any number of the at least one solution.

Some embodiments may further involve scraping the internet to identify additional information about the at least one solution. This second scraping may be separate from the first scraping to identify the at least one solution, or may be performed in conjunction with the first scraping. In other words, the solution and the additional information about the solution may occur through the same scaping, or may occur through multiple scrapings. For example, nested scrapings may be performed where an initial dataset is scraped and thereafter a more refined dataset is scraped from the initial dataset. The nestings can have more than two levels. Regardless, the second scraping may include identifying at least one of a proof of concept for the identified at least one solution; a degree of safety associated with the identified at least one solution; an economic feasibility associated with the identified at least one solution; a commercial applicability associated with the identified at least one solution, or any other information that may indicate an effectiveness, feasibility, or viability of the solution. As with the first scraping described above, performing the second scraping may include performing a semantic analysis on the scraped information, as described herein.

FIG. 6B is a block diagram illustrating an example, process 630 for recommending a solution 650 based on information scraped from source(s) 150, consistent with some disclosed embodiments. For example, this may include identifying solution 640 through a first scraping of sources 150, as shown in FIG. 6B. Process 630 may further include a second scraping of sources 150 for a proof of concept 642, a degree of safety 644, an economic feasibility 646, and/or a commercial applicability 648. In some embodiments, solution 640, proof of concept 642, a degree of safety 644, an economic feasibility 646, and a commercial applicability 648 may be identified as part of a single scraping process. Alternatively or additionally, a first scraping may be performed to identify solution 640 and an additional scraping may be performed to identify proof of concept 642, degree of safety 644, economic feasibility 646, and/or commercial applicability 648, as described above.

As used herein, a proof of concept for the identified at least one solution may be any information indicating that the identified at least one solution is effective in resolving a corresponding unmet technological need (which may be the same or similar to the current unmet technological need). For example, a proof of concept may include evidence or other information (such as a test run), that directly or indirectly indicates results of an experiment, solution implementation, or pilot project. Proof of concept 642 may be represented in the form of customer reviews, testimonies, users' ratings or votes, or other forms of user validation indicating a solution was effective from the perspective of a customer, patient, or other form of end user. As another example, the proof of concept may include evidence that the solution was implemented, which may be in the form of photographs or images, notes, results, schematics, or any other information showing the solution being implemented. As another example, the proof of concept may be in the form of test results related to the identified solution. For example, the proof of concept may include documented tests or experiments evaluating the solution, which may be performed by an entity that implements the solution or a third party. In some embodiments, a proof of concept may be the results of a partial or smaller-scale implementation of the solution. For example, the proof of concept may include the result of a pilot program, an experimentation, a market test, a prototype validation, a trial, or other forms of deployments of a solution for evaluation purposes. In some embodiments, the at least one proof of concept may include information indicating how the identified at least one solution can be executed. For example, this information may include a description or indication of at least one step or action taken to implement the identified at least one solution, and may be presented in the form of a publication, a user description, test protocols, training videos, or various other types of information that may indicate the solution can be executed. In the context of a clinical trial, for example, a proof of concept may be a document or other information showing that the clinical trial was completed (e.g., a Food and Drug Administration (FDA) submission).

As used herein, a degree of safety may refer to any indication of a potential harm that may arise from implementing the identified at least one solution or a likelihood of the potential harm occurring. The potential harm may be a harm to an individual or a harm to a group of individuals (e.g., a community, society as a whole, etc.). In some embodiments, the potential harm may be a physical harm, such as a health risk to one or more individuals. For example, if the solution is a particular form of treatment for a disease, the potential harm may represent risks or side effects of the treatment. As another example, if the solution is a construction of a building or development of a product, the potential harm may indicate a likelihood of workers being injured during the construction or development. In some embodiments, the potential harm may be a cybersecurity threat. For example, if the solution is implementation of a particular software, the degree of safety may be an indication of whether the software is likely to present a privilege escalation risk, a risk of exposing privileged credentials, or any other risk of exposing secure, sensitive, or confidential information. The degree of safety may include other forms of harm, such as damage to auxiliary equipment or structures, scheduling delays, environmental impacts, monetary damages, or any other form of adverse impact of a solution.

Degree of safety 644 may be indicated in the scraped information in various ways. In some embodiments, the degree of safety may be indicated by a description of an actual harm that resulted from implementing the solution. For example, a news article may indicate a number of injuries suffered or a data breach that occurred during implementation of the solution, or a publication may indicate negative side-effects resulting from a treatment. Alternatively or additionally, the degree of safety may be indicated by an accreditation or regulatory approval associated with the solution. For example, the degree of safety may be in indication of a regulatory approval or certification by the Food and Drug Administration (FDA), National Environmental Policy Act (NEPA), UL (formerly Underwriters Laboratories), National Sanitation Foundation (NSF), American National Standards Institute (ANSI), the European Commission (CE), European Medicines Agency (EMA), Energy Star™, or certifications from various other regulatory entities or third-party organizations. In some embodiments, the degree of safety may be indicated by evidence of one or more safety procedures followed during implementation of the solution.

An economic feasibility may refer to any indication of whether the solution is practical to implement from an economic standpoint. For example, economic feasibility 646 may be a degree to which the advantages of the solution outweigh the economic costs. Although a particular solution may be highly effective for resolving an unmet technological need, it may be prohibitive from a cost standpoint. Accordingly, the economic feasibility may include a cost/benefit analysis for implementing the identified at least one solution. Accordingly, the scraped information may include information comparing the economic costs with the advantages of the solution (which may be economic advantages or other advantages, such as resolving the unmet technological need). This information may be in the form of a detailed cost/benefit analysis or report prepared in association with the solution, or may be in the form of commentary or other text discussing the relative cost and/or benefits. As another example, the information may be in the form of customer reviews or testimonials indicating whether the solution is too expensive, a good value, etc. In some embodiments, the scraped information may include a cost associated with implementing the solution, and the cost/benefit analysis may be performed as part of the disclosed embodiments. For example, the scraped information may indicate a total financial cost for implementing the solution, which may be compared to a predetermined or desired value (e.g., a budget, goal, etc.) for resolving the unmet technological need.

As used herein, a commercial applicability may refer to a relative degree to which a product or service is worth trading in a marketplace for generating a profit. Commercial applicability may be distinct from economic feasibility. In other words, even if a product or service is economically feasible, it may not be commercially applicable if it is not appealing to the market and would not be desirable to customers. As one example, dried insect larvae may be a great source for protein and could be produced inexpensively, but it may not be commercially feasible if customers are reluctant to buy it. In some embodiments, the commercial applicability may include an indication of how effectively the identified at least one solution can be implemented in a particular industry. The particular industry may be the same industry or a different industry from an industry associated with the unmet technological need. For example, if the identified at least one association is associated with a different industry than the current unmet technological need, the commercial applicability may be an indication of how well the solution was implemented in the associated industry. This may provide insights into how applicable the solution is generally, which may provide some indication of how applicable it will be in the industry associated with the current unmet technological need. Alternatively or additionally, the previous solution may be a solution implemented in the same industry as the current unmet technological need, which may provide a more direct indication of the commercial applicability in this particular industry.

The commercial applicability information may be represented in various formats from the scraped data. For example, commercial applicability 648 may include news articles or other documents indicating the reception of the solution within an industry. In some embodiments, the commercial applicability may include statistics about implementation of the solution, including sales data, customer review or ratings data, social media posts indicating a user is associated with the solution (e.g., purchased a product, received a service, participated in a study, etc.), or any other form of data that may indicate the extent to which the solution was implemented.

Some embodiments of the present disclosure may further involve analyzing the identified at least one solution, and the at least one of the scraped proof of concept, the scraped degree of safety, the scraped economic feasibility, or the scraped commercial applicability to thereby recommend implementation of at least one specific solution from the identified at least one solution. The specific solution may be a solution identified through scraping the Internet, as described above. For example, process 630 (FIG. 6B) may include recommending solution 650, which may correspond to solution 640. In some embodiments, the recommended solution (e.g., solution 650) may be different in at least one aspect relative to the identified solution (e.g., solution 640). For example, some embodiments may involve recommending at least one modification to solution 640 in order to improve or tailor solution 640. As some examples, this may include modifying solution 640 to exclude an aspect that made solution 640 overly expensive, eliminate an aspect of solution 640 deemed unsafe, modify an aspect of solution 640 to make it more effective or commercially applicable, or the like. These modifications may be based on information scraped from the Internet, such as proof of concept 642, degree of safety 644, economic feasibility 646, and/or commercial applicability 648.

The analysis of the identified at least one solution, and the at least one of the scraped proof of concept, the scraped degree of safety, the scraped economic feasibility, or the scraped commercial applicability may include any form of statistical analysis, comparisons, algorithms, processing, or other manipulation of data to identify the at least one specific solution. In some embodiments, the analysis may include generating a score for one or more of the scraped proof of concept, the scraped degree of safety, the scraped economic feasibility, or the scraped commercial applicability. For example, a given solution may be scored with respect to one or more of the proof of concept, degree of safety, economic feasibility, and/or commercial applicability based on a degree to which the solution is determined to perform in each of these categories, as described in further detail above. The score may be represented in various forms, such as a percentage, a value within a range of values (e.g., 0-5, 0-10, 0-20, etc.), a text-based rating or indicator (e.g., “Excellent,” “Fair,” “Poor”), through one or more icons (e.g., a number of stars, a thumbs up, etc.), or various other indicators or values. In some embodiments, the score may be a binary score indicating whether the category of assessment (i.e., proof of concept, degree of safety, economic feasibility, and commercial applicability) is deemed to be acceptable or satisfied. For example, the scores may be a series of checkboxes indicating whether the solution has been shown to be effective, whether the solution is deemed safe, whether the solution is economically feasible, and whether the solution is commercially applicable. In some embodiments, this may include determining an initial score for each category of assessment in the form of a value and comparing the value to a predetermined threshold (which may be different for each category) and determining the binary score based on the comparison. In some embodiments, different formats of scores may be assigned to each category of assessment. For example, an economic feasibility may be represented in a dollar amount, a proof of concept may be a binary checkbox indicating the solution has been demonstrated to be effective or not, and a commercial applicability may be given a rating out of three stars.

The scores for each category of assessment may be aggregated in various ways. In some embodiments, a composite score for a particular solution may be generated, which may be an average of the scores for each category of assessment. Alternatively or additionally, the composite score may reflect a weighted average of the scores for each category of assessment. Accordingly, each category of assessment may be associated with different weights indicating an importance of the category. For example, for a particular industry, user, organization, or unmet technological need the commercial applicability may be more important than economic feasibility, and therefore may have a higher weight in determining a composite score. In some embodiments, the weight for each category of assessment may be specified by a user. For example, a user interface may be provided allowing a user to adjust the relative weights of each category of assessment. The composite score may be determined based on various other forms of statistical analyses or algorithms.

In embodiments where the at least one solution includes a plurality of solutions, the plurality of solutions may be ranked based on at least one of the proof of concept, the degree of safety, the economic feasibility, or the commercial applicability. In some embodiments, this may include ranking each of the plurality of solutions based on a composite score, as indicated above. Alternatively or additionally, the ranking may be based on various individual factors, including the proof of concept, the degree of safety, the economic feasibility, the commercial applicability, or any other relevant factors or combinations thereof. Some embodiments may involve filtering the plurality of solutions. For example, this may include filtering solutions that do not meet a threshold score for one or more of a composite score, a proof of concept score, a degree of safety score, an economic feasibility score, a commercial applicability score, or combinations thereof. In some embodiments, recommending implementation of the at least one specific solution may include selecting the at least one particular solution based on the ranking. For example, the at least one specific solution may be the solution having the highest composite score, a solution that meets a minimum requirement for each category of assessment, or the like.

According to some embodiments, the recommended implementation of at least one specific solution is further based on additional information. The additional information may be scraped from the internet similar to proof of concept 642, degree of safety 644, economic feasibility 646, and/or commercial applicability 648, or may be accessed from various other locations. In some embodiments, the additional information may be accessed from a database, such as database 112, or another database included in system 100. The additional information may include any information that may impact a determination as to whether a particular solution is feasible. In some embodiments, the additional information may include scheduling or timing information associated with a solution. For example, a solution that is otherwise feasible and satisfactory may be eliminated or ranked lower if the lead time for the solution prevents it from being implemented before a specified date. In some embodiments, the additional information may include an indication of available resources for implementing the solution. The indication of available resources may include a determined availability of personnel, financing, energy or power, computing power (e.g., processing bandwidth, memory, etc.), equipment, or various other resources that may be needed to implement a solution. Accordingly, determining the availability may include querying a data structure, querying a user or other entity, accessing a calendar or scheduling program, accessing a website or cloud platform, or various sources of information.

In some embodiments, the additional information may include an expertise of a querying individual. A querying individual may refer to an individual who submitted a request for the recommendation. For example, the querying individual may have provided the indication of the unmet technological need, as described above. The expertise of the querying individual may be any indication of a level of experience, a degree of knowledge, or a qualification of the individual. For example, the expertise may be determined based on a user input, based on stored profile information, based on accessing a social media or other web-based platform, or the like. This expertise may be used to tailor the recommendation to individual. For example, based on the level of expertise of the querying individual, solutions that are not within the skillset of the individual may be filtered out or ranked lower than solutions more squarely within the individual's capabilities. Accordingly, some embodiments may include determining or estimating a level of expertise associated with one or more solutions (including the at least one specific solution).

The implementation of the at least one specific solution may be recommended in various ways. In some embodiments, this may include transmitting a message indicating the recommended solution. For example, this may include transmitting data indicating the recommended solution from server 110 to computing device 120, to an account associated with user 130 (e.g., an email account, etc.), or various other forms of electronic transmission. As another example, the recommendation may be presented to a user via a graphical user interface. For example, computing device 120 may be configured to present the recommendation to user 130 through display 272.

FIG. 6C illustrates an example interface 660 that may be presented to a user (e.g., via computing device 120), consistent with some disclosed embodiments. In this example, the unmet technological need may be a need for access to a particular type of MRI machine and the system may scrape the Internet to identify a previous solution in which an entity purchased a new MRI machine or any kind of evidence of new implementation of MRI machine. In some embodiments, interface 660 may include an element 662 providing a description or summary of the recommended at least one specific solution. For example, this may be a text-based description of the scraped at-least one solution, which may be generated based on a semantic analysis or natural language processing technique applied to scraped information. In some embodiments, interface 660 may further include representations of one or more scores or ratings associated with a specific solution. For example, this may include displaying score 664, as shown in FIG. 6C. The score may be a composite score, a score associated with one or more of a proof of concept, degree of safety, economic feasibility, and/or commercial applicability, or various other scores or metrics described herein. In some embodiments, the recommendation may include one or more specific steps for implementing the recommended solution. For example, interface 660 may include an indication of a step 666 for implementing the recommended solution. The steps may be derived from scraped information describing how the solution was implemented previously.

As indicated above, in some embodiments, multiple solutions may be identified for a particular unmet technological need. In some embodiments, a user may be presented with multiple potential solutions and may select a solution from the potential solutions. In some embodiments, the user may (at least initially) reject a primary solution and request a different solution be presented. For example, interface 660 may include an element 668 through which a user can request a different solution be presented. In this example, a secondary solution may be to purchase a used MRI machine, to ask a medical facility to loan their MRI machine, or various other solutions. For example, this may cause interface 660 to present the solution with the next highest score or ranking. Alternatively or additionally, interface 660 may present multiple solutions simultaneously, which may allow a user to more easily compare two or more recommended solutions.

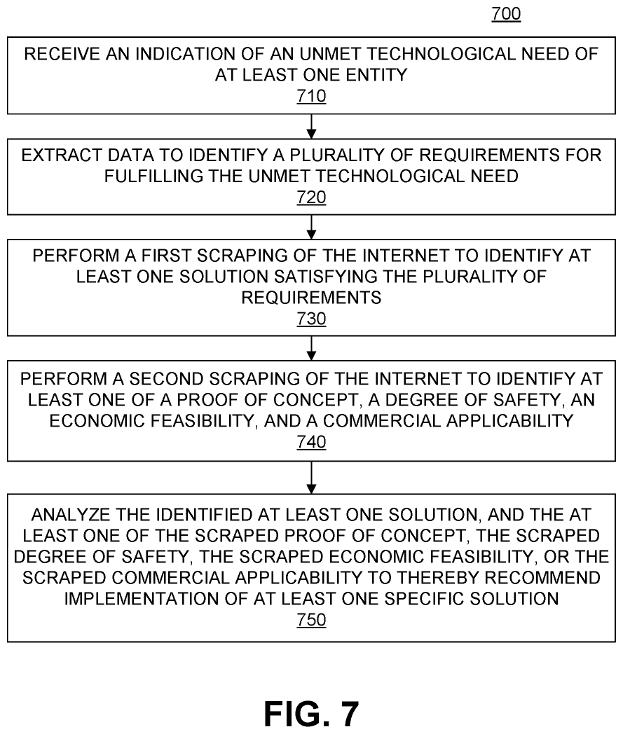

FIG. 7 is a flowchart showing an example process 700 for identifying solutions to unmet technological needs, consistent with some disclosed embodiments. Process 700 may be performed by at least one processor, such as processor 210. In some embodiments, a non-transitory computer readable medium may contain instructions that when executed by a processor cause the processor to perform process 700. Further, process 700 is not necessarily limited to the steps shown in FIG. 7, and any steps or processes of the various embodiments described throughout the present disclosure may also be included in process 700, including those described above with respect to FIGS. 6A, 6B, and 6C.

In step 710, process 700 may include receiving an indication of an unmet technological need of at least one entity, as described previously. In some embodiments, receiving the indication of the unmet technological need may include receiving an input through a user interface, such as interface 430.

In step 720, process 700 may include electronically extracting data from a data source to identify a plurality of requirements for fulfilling the unmet technological need. As indicated above, the requirements may include any form of specification, constraint, or condition that should be met in order to satisfy the unmet technological need, as described previously. In some embodiments, the data source may be a database or data structure, such as database 112. Alternatively or additionally, the data source may be an Internet-based resource. Accordingly, electronically extracting data from the data source may include scraping the Internet.

In step 730, process 700 may include performing a first scraping of the internet to identify at least one solution satisfying at least one of the plurality of requirements. As indicated above, the at least one solution may include one or more actions taken by an entity or a result achieved through the one or more actions. In some embodiments, the at least one solution may include a plurality of solutions. As described above, the first scraping may include performing semantic analysis on scraped information.

In step 740, process 700 may include performing a second scraping of the internet to identify at least one of: a proof of concept for the identified at least one solution; a degree of safety associated with the identified at least one solution; an economic feasibility associated with the identified at least one solution; and a commercial applicability associated with the identified at least one solution. In some embodiments, performing the second scraping includes scraping a plurality of media items, as described previously. For example, this may include scraping news articles, publications, social media posts, customer or product reviews, videos, images, or other information that may provide information about a solution. In some embodiments, step 740 may further include performing semantic analysis on scraped information.

As described above, the at least one proof of concept may include information indicating how the identified at least one solution can be executed. For example, this may include customer reviews, results of an experiment or test, description of a pilot program, or any other form of evidence that indicates the solution is effective for resolving an associated issue. The degree of safety may include an indication of potential harm that may arise from implementing the identified at least one solution. For example, the potential harm may include a cybersecurity threat, a physical injury, a health risk, or other harm that may result from the solution. The economic feasibility may include a cost/benefit analysis for implementing the identified at least one solution. Accordingly, the economic feasibility may be ascertained from cost information, pricing information, a cost/benefit analysis, or other financial information associated with the solution. As described above, the commercial applicability may include an indication of how effectively the identified at least one solution can be implemented in a particular industry. For example, the commercial applicability may relate to how likely an industry or audience is to adopt or accept the solution, or any obstacles that may hinder the solution from being implemented.

In step 750, process 700 may include analyzing the identified at least one solution, and the at least one of the scraped proof of concept, the scraped degree of safety, the scraped economic feasibility, or the scraped commercial applicability. Based on the analysis, step 750 may include recommending implementation of at least one specific solution from the identified at least one solution, as described previously. The analysis in step 750 may include any form of combined analysis on the scraped information. In some embodiments, step 750 may include generating a score associated with each of the at least one of the scraped proof of concept, the scraped degree of safety, the scraped economic feasibility, or the scraped commercial applicability. In some embodiments, this may further include generating a composite score based on the scores for the scraped proof of concept, the scraped degree of safety, the scraped economic feasibility, or the scraped commercial applicability. In embodiments where, the at least one solution includes a plurality of solutions, step 750 may include ranking the plurality of solutions based on at least one of the proof of concept, the degree of safety, the economic feasibility, or the commercial applicability. Accordingly, recommending implementation of the at least one specific solution may include selecting the at least one particular solution based on the ranking In some embodiments, the recommended implementation of at least one specific solution may further be based on additional information, which may include at least one of an indication of available resources or an expertise of a querying individual. For example, the additional information may be accessed from a database. In some embodiments, the recommendation may be presented through a user interface, such as interface 660.

It is to be understood that embodiments disclosed herein may be applied to a wide range of unmet technological needs. Various use cases for identifying solutions to unmet technological needs are provided below. These use cases are provided by way of example and are not limiting of the present disclosure in any way. As one particular example, the unmet technological need may include a resource needed for conducting a clinical trial. For example, this may include a specified piece of equipment or an individual with a specified skillset needed to conduct the trial. In some embodiments the resource needed for conducting the clinical trial may be identified based on a least one trial protocol parameter. For example, receiving the indication of the unmet technological need (step 710) may include accessing a document indicating protocol parameters for the clinical trial and determining that an unmet technological need exists with respect to a parameter defining a resource needed for the trial. In some embodiments, the resource needed for conducting the clinical trial may be identified based on application of a trained machine learning model to the least one trial protocol parameter. For example, a model may be trained to identify resources needed for a clinical trial based on training data, which may include a set of clinical trial protocol documents with indicators of associated resources needed to complete the clinical trial.

In this example, the plurality of requirements may indicate a particular type or characteristic of resource that is needed. For example, this may include a level of expertise associated with an individual or a particular aspect of a piece of equipment (e.g., size, capacity, type, etc.). As another example, a requirement may define a time deadline or a budgetary cost associated with the resource. These requirements may be extracted electronically from a database or other data source (step 720). Disclosed embodiments may include scraping the Internet to identify previous solutions associated with the resource (step 730). In this example, the previous solutions may include indications of other clinical trials requiring the same resource and how the resource was acquired. Disclosed embodiments may further include performing a second scraping of the internet to identify at least one of a proof of concept; a degree of safety; an economic feasibility; and a commercial applicability (step 740). In this example, a proof of concept may include an indication that a previous clinical trial was completed successfully. The degree of safety may include any side effects or injuries that may have arisen through use of the identified resource. For example, if the recommended solution includes purchasing a used MRI machine and moving it to a testing facility, this may impose higher safety risks than purchasing a new MRI machine and having it installed professionally. An economic impact may include a cost of purchasing the resource compared to the benefits the resource provides. For example, the purchase of a new MRI machine may be relatively expensive but may work better than a used MRI machine that is more affordable. The commercial applicability may include how many individuals are willing to use the MRI machine. For example, some patients may experience claustrophobia and may need MRI machines with larger cavities for receiving the patient, or the like.

Disclosed embodiments may include analyzing the information to recommend implementing a particular solution (step 750). For example, where the resource needed for conducting the clinical trial includes a specified piece of equipment, the at least one specific solution may include an identification of at least one of a supplier, an operator, or a funding source for the specified piece of equipment. As another example, where the resource needed for conducting the clinical trial includes an individual with a specified skillset, the at least one specific solution may include an identification of at least one of the individuals having the specified skillset, a method for training an individual to obtain the specified skillset, or an individual qualified to train an individual to obtain the specified skillset.

In some embodiments, this process may be repeated for various other resources, such as identifying a supplier, funder, operator, trainer, staff, or other individuals to conduct the clinical trial. In some embodiments, an unmet technological need may be defined as a need for specific training, knowledge, or best practices for conducting the trial. For example, this knowledge may include medical training, procedures, equipment operation experience, knowledge of regulatory approvals, or various other applicable knowledge. In some embodiments, the unmet technological need may be broken down further to finding specific resources associated with gaining the knowledge or training. For example, a separate unmet technological need may be defined for finding a source of the knowledge, finding a trainer, finding a training method, and finding staff to be trained. Accordingly process 700 may be applied to each of these smaller unmet technological needs.

As another potential use case, process 700 may be applied in the agriculture industry to prepare for growing a new crop. For example, a farm may be planning to grow a new crop intended for human consumption and therefore should not be in contact with harmful chemicals. Accordingly, an unmet technological need may be defined as a solution to protect the crop from a variety of pests, with strict requirements for the method to be safe for the consumption of the product and environmentally friendly. Disclosed embodiments may be used to scrape the Internet for information mentioning solutions to this need, mentions of more detailed requirements, indications of other farms/users that have expressed similar needs, people who answered them (e.g., in a discussion board, through customer reviews, etc.), and suppliers of products. The scraping may result in information such as identifications of researchers, suppliers, farmers, implementation instructions or protocols, customers' complaints, users' responses, and regulatory issues including specific standard requirements. The scraping may also identify locations a solution has been implemented, characteristics of soil and climate, degrees of safety, prices, commercial solutions (if available), people with expertise in applying the product, or various other information. By aggregating this information, multiple suppliers, funders (if relevant and available), operators, trainers, costs analysis, and precautions may be identified. Since the solution may be complex, there may be a necessity for training. Some embodiments may facilitate communication channels to allow the farmer to connect with other farmers to discuss their experience with various solutions.

As another potential use case, process 700 may be implemented in association with development of a technological or market innovation. In developing any new product, system or service it is beneficial for an organization to understand the ‘state of the art.’ Accordingly, an unmet technological need may be defined to develop a particular product or service, which may be defined by various stakeholder requirements, industry requirements, customer requirements, or the like. Disclosed embodiments may include scraping the internet to break down the aspects or dimensions (i.e. location, users, buyers, current solution, pricing, market trend, competition, IP issues etc.) and, for each dimension, find and provide relevant information to inform decisions (e.g. investment; trial market, a go/kill decision, etc.). The outcome of the assessment of each dimension will inform a plan for further development. For example, if the analysis shows that there is a quantifiable market need but not technical solution yet, the solution may be to develop technical aspects of the product or service. If, on the other hand, the analysis indicates that there are several concepts with favorable validation results, the recommended solution may be to implement a pilot application or trial study.

In view of the above, embodiments consistent with the present disclosure provide a significant improvement over existing systems. For example, a user may select a particular unmet technological need and receive a recommendation for resolving the unmet technological need, which may be informed by information scraped from a wide variety of sources. In particular, information may be scraped from a wide variety of current sources, such as news articles, publications, social media platforms, or other sources, and may be analyzed to develop a solution. This aggregation from multiple different sources provides a more robust and current assessment of available solutions and can result in a combination of multiple individual solutions to reach an optimal and tailored solution to the current unmet technological need. Accordingly, embodiments consistent with the present disclosure provide, among other advantages, improved accuracy, efficiency, convenience, and functionality over conventional techniques.

As described throughout the present disclosure, some embodiments may include identifying unmet technological needs and facilitating the resolution of the unmet technological needs. For example, this may include identifying entities having roles or skills suitable for collaborating to resolve an unmet technological need. In many instances, separate entities may be interested in the same goal but may not have a way of connecting. This may be especially true, for example, in remote work environments, where entities may be distributed across a state, country, or globally. Even in local regions, however, given the sheer volume of data that is available, locating and connecting entities in an efficient manner can be difficult if not impossible. It may be even more difficult in highly competitive markets when fast and effective responses to hundreds or thousands of competing inputs may be needed in a short period of time. As another example, during a pandemic, information (e.g., symptoms) may need to be analyzed quickly to develop a preventative or therapeutic response. Accordingly, solutions are needed for bringing together entities that may have the same interests and may be able to accomplish a task based on the interest. Additionally, projects and goals may change over time. New entities may be needed to accomplish new goals and/or some entities may no longer be needed to accomplish a goal. Accordingly, solutions are also needed to find, approach, and commit entities with specific expertise according to the shifting requirements of a development project.

Some embodiments disclosed herein address these and other issues by dynamically forming social clusters based on overlapping interests in contributing to a common purpose. This may include processing data associated with the identities to determine overlapping interests, which may form the basis for the ephemeral groups. This data may be scraped from the Internet to provide a much more robust and efficient search than conventional techniques. As individuals' needs or interests change, groups can be dissolved and others can be formed dynamically. Additionally or alternatively, individuals may be added to or removed from a group.

Some disclosed embodiments provide solutions for forming ephemeral social clusters. As described herein, an ephemeral social cluster may refer to a temporary group of people, entities, and/or algorithms, that was created for a specific reason or objective and for a specific period of time. In other words, an ephemeral cluster may be a temporal relationship between a group of humans, entities or algorithms based on at least one commonality. For example, one or more individuals who may be of different backgrounds, geographies, and skillsets, may be working on the same objective (e.g., reducing alcoholism in their respective neighborhoods, etc.), and may be temporarily grouped into an ephemeral social cluster based on the common objective. Clusters may be formed by bringing together groups of people, entities, and/or algorithms. Clusters may be formed for many purposes. For example, clusters may be formed to solve an environmental problem, to perform scientific research, to develop agricultural techniques, or for any other reason, including the various examples of unmet technological needs provided throughout the present disclosure.

Some disclosed embodiments may involve scraping the internet for commonality data identifying a plurality of entities associated with a commonality. As described throughout the present disclosure, scraping the internet may include collecting information from websites using various data extraction techniques. Data may be scraped from social platforms, discussion groups, news outlets, company websites, or any other type of website. The data may then be stored in a central database or location. The data may be stored in CSV format, Excel format, JSON, or any other type of storage means, which may allow the data to referenced in an effective and efficient manner Web scraping may be executed by software. The internet may be scraped to search for commonality data. A commonality may be an interest or attribute shared by entities, such as a person, company, institution, or any other type of thing that may have an interest in the same thing. Data may include information related to a commonality. For example, a commonality may be an interest in scientific research or various other types of interests. Data related to scientific research may include a clinical trial for a new drug. As another example, a commonality may be a geographical location. Data related to a geographical location may include job openings in the location, availability of entities to collaborate with others in the region, locations of facilities or other resources in the region, or other location-based information. Multiple entities may be connected to a specific commonality and identified. For example, commonality data may be job openings in a geographical location and the plurality of entities may include entities searching for a job in that geographical location, entities meeting one or more requirements for a job opening, or similar commonalities. The data identifying a plurality of entities may include text, images, sounds, audio, videos, or other types of information. The data identifying a plurality of entities may be received by a client-side computing device, a host computer, a server, a network or any other type of device used to receive information. Such data may be received from a website, a server, a network, a computer, or any other type of device used to send data.

In some embodiments, the plurality of entities are subscribers to at least one platform. A platform may be program, aggregation of computer code, channel, mechanism, or any other instrumentality that enables communication between participants. Such a platform may enable users to participate in and receive updates. A platform may be a website, network, virtual community, or other type of system. For example, a plurality of entities may be subscribers to a platform that distributes information about new cancer drugs. As another example, a plurality of entities may be subscribers to a chat room that discusses environmental challenges. Other example, platforms may include social network platforms (e.g., Facebook™, Snapchat™, etc.), professional databases or networks, regional or local networks, or any other platforms that may enable or facilitate communication among its subscribers.

Some disclosed embodiments may include performing electronic semantic analysis on the scraped commonality data to identify a subset of the plurality of entities having at least one specific overlapping interest in contributing to a common purpose. As described herein, semantic analysis may refer to any form of analysis or processing of natural language (in text form) to draw meaning and/or context. Semantic analysis may include relating syntactic structures from phrases, clauses, sentences, paragraphs, complete writings, to their language-independent meanings. In some embodiments, semantic analysis may include parsing elements of text and assigning each a grammatical role. The structure may then be analyzed to remove ambiguity from any word with multiple meanings. As one example, semantic analysis may include analyzing sentences and sequences of words by using a set of rules, principles, and processes in order to determine a stage and development progression of research, as described below. Semantic analysis may be executed by a computer, machine learning algorithm, or any other type of electronic system, and may be applied to scraped commonality data, described above. Semantic analysis may be used to identify a subset of the plurality of entities. The subset may be a smaller group of the plurality of entities that may have overlapping interests. Overlapping interests may include concerns, objectives, endeavors, projects, vocations, passions, and/or parts of a common goal that are shared by the subset. In some embodiments, the interest may be related to a common purpose. In this context, a common purpose may refer to a particular goal or objective that is at least partially shared by a plurality of entities. A common purpose may include an interest in contributing to a specific cause, solving an unmet need, helping a specific group of people, and/or helping in a specific condition or situation, e.g. car accidents, war, pandemic, or any other type of interest. For example, a common purpose of the subset may include an interest in participating in a clinical trial. As another example, the internet may be scraped to determine a plurality of entities that may be interested in research articles. A common purpose may include an interest in peer reviewing research articles. Semantic analysis may be performed to identify a subset of entities that are interested in peer reviewing research articles from the plurality of entities that are interested in research articles. The interest may be expressed directly and/or may be determined using semantic analysis based on historical data. Another example may include the desire to help healthcare professionals in underserved communities. While various examples are provided herein, it is to be understood that a common purpose may include a wide variety of goals or objectives shared by a subset of individuals.

In some disclosed embodiments, performing electronic semantic analysis on the scraped commonality data may include performing initial semantic analysis to identify the subset of the plurality of entities, and performing a subsequent semantic analysis to identify potentially available entities within the subset of the plurality of entities. Initial semantic analysis may include semantic analysis that may be performed at a first instance on scraped information. In this case, the initial semantic analysis may determine the subset of plurality of entities. Subsequent semantic analysis may be performed after the initial semantic analysis and thus may include analysis that occurs after the first instance. Subsequent analysis may be used to determine entities within the subset that may be interested in a more defined interest. For example, a commonality data may include an interest in investing in a certain project. Initial semantic analysis may be performed to determine a subset of a plurality of entities that are interested in investing in a certain project. Subsequent semantic analysis may be performed to determine potentially available entities within the subset that may be able to invest in a certain project at the moment analysis is performed. Semantic analysis may determine the group of potentially availably entities by looking for words such as “available”, “ready to invest”, or other terms that indicate an entity is available. For example, an entity may ask certain questions or use a specific sequence of words to indicate the entity may be an active investor. As a further example, an entity may ask in a specific social group about a lawyer that could help in an investment due diligence process. Semantic analysis may be performed based on a map of correlations between elements that were created, for example, from analyzing historical records and/or machine learning based on human selection and feedback, to determine that the entity may be an active investor. As another example, an availability of entities may be based on a status indicator, which may include any information indicating whether an entity is available to contribute toward a common goal. For example, a driver in a taxi or ridesharing service may be associated with an electronic status indicating whether they are available and/or willing to take a rider, a physician may post an indicator of whether they are taking new patients, or a website for a small construction company may indicate whether they are currently under contract for a project. Various other forms of information indicating whether an entity is available, ready, willing, or able may be used to identify the subset of entities that are potentially available.

Some disclosed embodiments may involve transmitting electronic communications to at least some entities of the subset of the plurality of entities. Electronic communications may include any form of communication that may be broadcast, transmitted, stored or viewed using electronic media, such as computers, phones, email and video. The communication may be transmitted through phone calls, faxes, text messages, video messages, emails, internet messaging, a notification within an app or application, or any other type of electronic means. In some embodiments, transmitting electronic communications may include transmitting a signal over a network, such as network 140. For example, server 110 may transmit electronic communication to computing device 120. Electronic communications may include any transfer of signs, signals, writing, images, sounds, data, or intelligence of any nature transmitted in whole or in part by wire, radio, electromagnetic, photo electronic, or photo-optical system that affects interstate or foreign commerce. Electronic communications may be sent to some of the entities of the subset of the plurality of entities. Additionally, electronic communications may be sent to all of the entities of the subset of the plurality of entities. For example, an email may be sent to at least some entities. As another example a text message may be sent to at least some entities.

In some embodiments, the electronic communications may include an invitation to contribute to the common purpose. Accordingly, the electronic communications may include an indication or description of the common purpose. An invitation may include a request or offer to do something, which may include a request or offer to contribute to the common purpose generally, or a request or offer to do a particular task, serve a particular role, or any other activities. An invitation may be sent through electronic means, such as email, text, video. For example, an electronic communication may include an email invitation to participate in a clinical trial for a cancer drug in order to establish safety data on the drug. As another example, an electronic communication may include a text message to invest money in a specific medical waste company to contribute to reducing medical waste. In some embodiments, the invitation may include an indication of a reason the entity is believed to share an interest in contributing to the common purpose, which may be generated automatically.

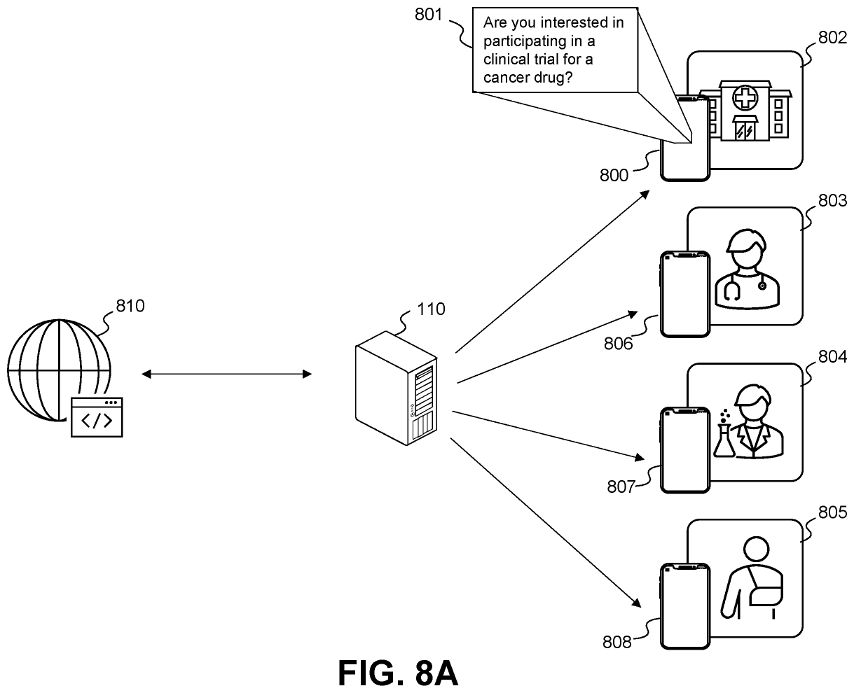

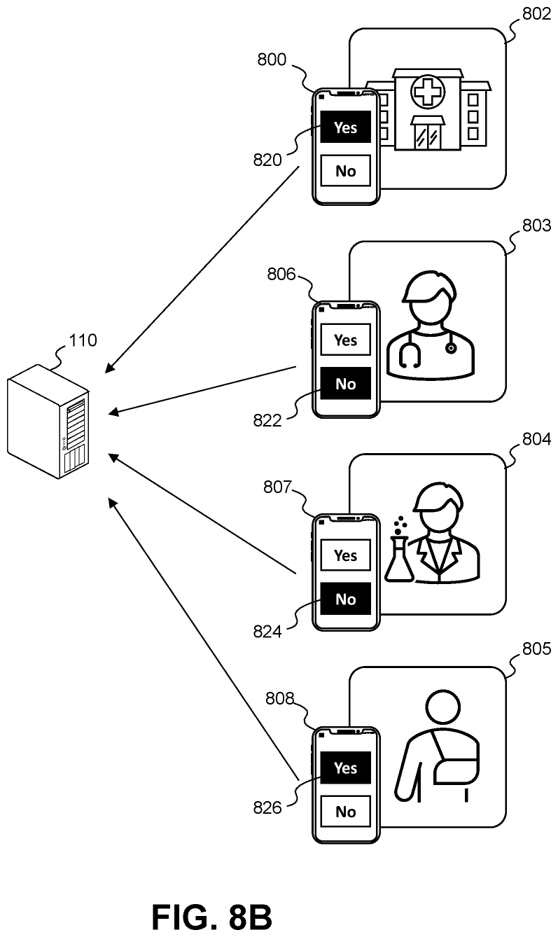

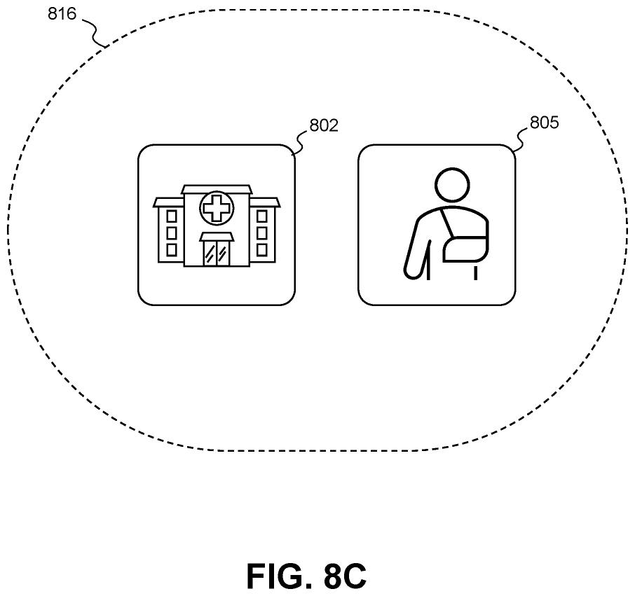

By way of example, FIG. 8A is a diagrammatic representation of an exemplary system for transmitting electronic communications to at least some entities. For example, as illustrated in FIG. 8A, server 110 may scrape internet 810 for commonality data to identify a plurality of entities associated with an interest in clinical trials. Server 110 may perform semantic analysis to identify a subset of entities that may be interested in participating in a clinical trial. Server 110 may transmit electronic communication 801 through electronic devices 800, 806, 807, and 808 to at least some entities 802, 803, 804, and 805. At least some entities may include a hospital 802, a doctor 803, a lab technician 804, and a trial participant 805. Electronic device 800 may be associated with entity 802, electronic device 806 may be associated with entity 803, electronic device 807 may be associated with entity 804, and electronic device 808 may be associated with entity 805. Electronic communication 801 may include an invitation to contribute to a common purpose, such as participating in a clinical trial for a cancer drug.

Some disclosed embodiments may involve receiving electronic responses to at least some of the electronic communications. Electronic responses may include electronic message received as a reply to a previous electronic message. Electronic responses may include any form of communication such as text, email, voicemail, instant messaging, video calls, or any other type of messaging, including those described previously. Electronic responses may be received in the form of signals by a client-side computing device, a host computer, a server, a system or any other type of device used to receive information. Such messages may be received from a website, a server, a network, a computer, or any other type of device used to send messages. Electronic messages may be received as a reply to an electronic communication, described above. For example, an electronic communication may be transmitted as an email. An electronic response may be received in the form of an email. As another example, an electronic communication may be transmitted as a text message. In some embodiments, an electronic response may be received in form of a voicemail. In some embodiments, receiving electronic responses may include providing a graphical user interface allowing a user to input an indication of a response. For example, transmitting the electronic communication may include displaying an interface inviting an entity to participate in achieving the common purpose, and the electronic response may be received through a selection by the entity to either accept or decline the invitation.

By way of example, FIG. 8B is a diagrammatic representation of an exemplary system receiving electronic responses to at least some of the electronic communications. For example, as illustrated in FIG. 8B, server 110 may receive electronic responses 820, 822, 824, and 826 from electronic devices 800, 806, 807, and 808 in response to electronic communication 801. Electronic responses 820, 822, 824, and 826 may indicate entities 802, 803, 804, and 805 interest in contributing to a common purpose, such as participating in a clinical trial for a cancer drug. Hospital entity 802 may respond “YES” through electronic device 800. Doctor entity 803 may respond “NO” through electronic device 806. Lab technician 804 may respond “NO” through electronic device 807. Trial participant 805 may respond “YES” through electronic device 808.

Some disclosed embodiments may involve, based on the received responses, generating an interest group defined by the at least one specific overlapping interest. For example, the interest group may include entities from the subset of the plurality of entities that indicate an interest or approval to join the group based on the electronic responses. An interest group may be a group of people, organizations, business, or other types of groups, that may be connected based on a common interest or concern. An interest group may be generated by creating a database or list of the entities interested in the common concern. For example, an overlapping interest may be peer reviewing research articles. An email may be sent to a subset of entities interested in peer reviewing research articles. Some of the entities in the subset may respond to the email with a text message. An interest group of entities interested in peer reviewing research articles may be created that includes the entities who responded to the email with a text message. In some embodiments, generating the interest group may include generating an interest graph, where each of the entities are represented by nodes on the graph and links between the nodes indicate a connection in association with the overlapping interest. In some embodiments, the links may include a label or other information identifying the overlapping interest. In some embodiments, the overlapping interest or common purpose may be represented as a node, and generating the interest group may include generating a links between nodes representing entities and the node representing the overlapping interest or common purpose.

By way of example, FIG. 8C is a simplified diagrammatic representation of an interest group generated based on the received responses. Interest group 816 may be formed based on electronic responses 820, 822, 824, and 826. Entities that respond “YES” to electronic communication 801 may be formed into interest group 816. Interest group 816 may include hospital 802 and trial participant 805.

Some embodiments may include subsequently scraping the internet to identify at least one new entity for inclusion within the subset of the plurality of entities. The internet may be constantly scraped, as described above, to search for new entities that may be interested in a common purpose. A new entity may be an entity that was not previously part of the subset of plurality of entities but may later be added to the subset after subsequent scraping. For example, a common purpose may include an interest in developing new compounds with medical properties. Scientists participating in developing the new compounds may be ready to perform clinical trials with the new compound. Subsequent scraping may occur to determine new entities that may be able to help develop the clinical trial. New entities that are identified, such as a regulatory expert, may be added to the subset of entities interested in developing new compounds.

Some embodiments may include sending an additional electronic communication to the at least one new entity and, based on a response to the additional electronic communication, include the at least one new entity in the interest group. A new entity may be sent an electronic communication, as described above. A new entity may also respond to the electronic communication through an electronic response, as described above. A new entity who responds to an additional electronic communication may be added to an interest group. Using the example described above, a regulatory expert may be identified as a new entity. The regulatory expert may be sent an email or other electronic communication asking if the expert is interested in developing a clinical trial for a new drug. The regulatory expert may respond electronically, indicating the expert's interest in developing the trial. Based on the response, the expert may be added to an interest group.

Some disclosed embodiments may involve causing the interest group to be stored in memory, wherein the stored interest group includes information identifying selected entities from the subset of the plurality of entities. The interest group may be stored in a data structure, hard drive, or any other type of structure capable of storing information. The interest group may be stored as a list, table, spreadsheet or any other type of format. In some embodiments, the interest group may be stored in a blockchain format such that the information is stored in a decentralized manner. The stored interest group may include information related to the entities that responded to the electronic communication. Information may include email addresses, phone numbers, home addresses, business addresses, or any other type of identifying information related to the selected entities. For example, the stored interest group may include entities interested in participating in a clinical trial for a cancer drug. The stored interest group may include the email addresses of the entities interested in the clinical trial.

In some embodiments, the common purpose may include conducting a clinical trial, as described throughout the present disclosure. In this example, each of the selected entities may be associated with at least one skill for conducting the clinical trial. A skill may include the ability to do something. A skill may be identified for a common purpose. For example, a common purpose may be conducting a clinical trial on a cancer drug Skills needed for the clinical trial may include a lab technician who can draw blood, a doctor who can monitor patients, a nurse who can administer a drug, or any other type of skill. Each selected entity may be associated with a skill. For example, a nurse may be selected who may be able to administer drugs.

As another example, a common purpose may include an interest in growing protein-rich crops in areas with water scarcity. Skills related to this purpose may include farming, ability to operate machinery, crop management skills, or other types of skills related to agriculture. Selected entities may be entities that may be able to perform these skills.

Some disclosed embodiments may involve receiving electronic data from a plurality of differing sources to thereby monitor the at least one specific overlapping interest of each of the selected entities and to determine that the overlapping interest is reduced for a specific one of the selected entities. An overlapping interest for the specific entity may be considered reduced in a variety of ways. In some embodiments, the interest may be reduced if an entity has lost interest in achieving the common purpose. For example, the entity may no longer post about a topic or may post information indicating a negative interest in a topic. Internet scraping may be performed, or previously identified data sources may be scoured, to determine a reduction in interest. For example, an identifier of the entity (e.g., an entity name may be used as part of the scraping our scouring process to locate information related to an entity's reduced interest. To facilitate such a determination, semantic analysis may be performed, as discussed earlier. As another example, one member of the group may provide an indication that another member of the group has lost interest in the common purpose. In some embodiments, the interest may be reduced based on the ability of the entity to contribute in an effective manner. For example, reduced interest may be determined if the entity failed to timely complete a task assigned to the entity, the entity no longer has a certain skill needed to contribute, the entity is no longer available (e.g., has become injured, has moved, has accepted a new position, etc.), or any other indication the entity has a reduced ability to contribute. As another example, the interest may be reduced if one or more tasks are completed. For example, the entity may have a specific role and may have fully completed his role. The reduced interest may be determined based on a determination that an assigned task is already accomplished, that the common purpose or goal has been accomplished, that a smart contract is fulfilled, that a proof of transaction has been received; based on data received from a third party that a task is complete, or any other indicators that the entity has completed one or more activities that would reduce an interest in contributing further.

In this context, electronic data may include any type of information exchanged via electronic means. Electronic data may be received from external and internal sources. External sources may include any existing websites, scholarly publications, news sources, professional/trade sources, books, conference proceedings, government documents, theses, dissertations, databases, including paid databases, or any other type of source. Internal sources may include data supplied by users, data learned from user behavior, or any other type in source. In some embodiments, the electronic data may be scraped from the Internet, as described throughout the present disclosure. Electronic data may be received from a source in order to monitor an overlapping interest of the selected entities. For example, an overlapping interest may include picking up trash along a specific beach. Data related to this interest may include dates when entities pick up trash along the beach. Data may be received from a local beach website or from a discussion group that schedules days to pick up trash. In some embodiments, the electronic data from a plurality of differing sources may include electronic data stored in a data structure. A data structure consistent with the present disclosure may include any collection of data values and relationships among them. The data may be stored linearly, horizontally, hierarchically, relationally, non-relationally, uni-dimensionally, multidimensionally, operationally, in an ordered manner, in an unordered manner, in an object-oriented manner, in a centralized manner, in a decentralized manner, in a distributed manner, in a custom manner, or in any manner enabling data access. By way of non-limiting examples, data structures may include an array, an associative array, a linked list, a record, a union, a tagged union, a binary tree, a linked list, a balanced tree, a heap, a stack, a queue, a set, a hash table, an ER model, a graph and a knowledge graph. For example, a data structure may include an XML database, an RDBMS database, an SQL database or NoSQL alternatives for data storage/search such as, for example, MongoDB, Redis, Couchbase, Datastax Enterprise Graph, Elastic Search, Splunk, Solr, Cassandra, Amazon DynamoDB, Scylla, HBase, TypeDB and Neo4J. A data structure may be a component of the disclosed system or a remote computing component (e.g., a cloud-based data structure). Data in the data structure may be stored in contiguous or non-contiguous memory. Moreover, a data structure, as used herein, does not require information to be co-located. It may be distributed across multiple servers, for example, that may be owned or operated by the same or different entities. Thus, the term “data structure” as used herein in the singular is inclusive of plural data structures. Thus, a data structure may include any collection of data values and relationships among them, and functions or operations that may be applied to the data. For example, an organization may maintain a local database storing information about contributions or interests of entities within the group and the reduced interest may be based on information from the database.

In some embodiments, receiving electronic data from a plurality of differing sources includes scraping local data structures. A local data structure may include a data structure, as defined above, that is present on a local computer or network. The local data structure may be scraped, as defined above. As another example, a data structure may be stored on a remote computing system, such as a cloud-based data structure.

In some embodiments, the information stored in the data structure includes status information. Status information may include any information that is generated in relation to an activity e.g., log data, system reports, or any other type of information. The status information may include any information that may indicate a reduced interest, such as a status indicating whether an entity is available, or any of the various information described above relating to a reduced interest. For example, the status information may include a system report of doctors that may be associated with a specific hospital. Another example may include logging data to determine that a user may be performing different activities and may have different commitments, so the user may no longer be available or may have a low interest.

The selected entities interested in an overlapping interest may change over time. The entities may change based on a reduction in the overlapping interest. The overlapping interest may be narrowed so that specific entities may no longer be enthusiastic about or focused on the interest. For example, as indicated above, an assigned task related to the interest may be accomplished, a task related to the interest was not timely fulfilled, a goal related to the interest may be accomplished, a smart contract related to the interest may be fulfilled, proof of a transaction related to the interest may be received, data may be received from a third party indicating a task related to the interest is complete, data may be received from an entity indicating reduced interest of another entity, or any other type of information that may indicate a specific entity is no longer part of the selected entities. As a specific example, an overlapping interest may include growing vegetables on rooftops within cities. An assigned task within the interest may include the need for someone to water the vegetables. The task may be assigned to an entity within the selected entities of the subset of the plurality of entities. After that task is assigned, specific entities that were also interested in watering the vegetables may no longer be interested in the overlapping interest. As another example, once the vegetables have been harvested, individuals assigned to watering the plants may no longer be needed. In some embodiments, additional individuals may then be added based on the change in status or a detected event. For example, drivers capable of distributing the vegetables may be added to the group.

In some embodiments, the determination that the at least one specific overlapping interest may be reduced by the specific one of the selected entities is based on a determination that the specific one of the selected entities completed a role associated with the common purpose, as described above. A role may include the function assumed or task committed to by a person or thing in a particular situation. A role may be completed when the function has been accomplished or the task has been performed. For example, a driver may complete their role of driving when they have brought patients to a center. As another example, a lab may complete their role when it has completed a test and delivered a result. Upon completion of a role, at least one specific overlapping interest may be reduced to no longer include the specific entity that has completed its role. Using the example above, a common purpose may include growing vegetables and an overlapping interest may include growing vegetables on rooftops within cities. A task of planting the vegetables may be assigned as a role to a specific entity who lives in the building. Once the entity has planted the vegetables, the role may be completed, and the interest may be reduced by no longer needing an entity to plant the vegetables.

In some embodiments, the determination that the at least one specific overlapping interest is reduced by the specific one of the selected entities may be based on a determination by the at least one processor that the specific one of the selected entities failed to timely complete a task associated with the common purpose. Failure to complete a task may mean that an entity may be unsuccessful in achieving a goal. Failing to complete a task may include not performing the task in a required amount of time, not performing an assigned role, performing the task incorrectly, or any other type of failure. Upon failure of a task, at least one specific overlapping interest may be reduced to no longer include the specific entity that failed to complete a task. Using the example above, a common purpose may include growing vegetables and an overlapping interest may include growing vegetables on rooftops within cities. A task of planting the vegetables by a certain date may be assigned to a specific entity who lives in the building. The entity may not plant the vegetables by that date and fail the task. After the date has passed and the entity has failed to plant the vegetables, the interest may be reduced to no longer include the entity that failed.

Some embodiments may involve suggesting an action to the at least one selected entity. An action may include an activity that may need to be performed by the at least one selected entity. In some embodiments, the suggested action includes executing at least one of an electronic nondisclosure agreement or an electronic engagement contract. For example, at least one selected entity may be asked to sign an agreement to prevent the entity from discussing the work the entity performed for a common purpose. In another embodiment, the suggested action includes generating terms of a smart contract. The terms may include any provision in a contract, including the parties, purpose of the contract, duties, timeline, or any other contract provision.

Some embodiments may involve receiving electronic data from a plurality of differing sources includes scraping the internet for information about the selected entities and ascertaining reduced interest from the scraped information. Electronic data may be scraped from the internet, as described above. Data may be scraped from a plurality of different sources, including external and internal sources, described above. The internet may be scraped to find information about the selected entities to determine a reduced interest. A reduced interest may include lowering the number of selected entities that are interested in a specific overlapping interest. For example, a specific overlapping interest may include participating in a clinical trial for a vaccine. An article may be scraped from the internet that includes information about a hospital, that was previously interested in participating in a clinical trial for a vaccine, is closing. A hospital may no longer be interested in or relevant for participating in a clinical trial and the interest may be reduced. As another example, a service provider that was previously interested in a vaccine clinical trial may begin participating in online activities, such as social groups, discussion rooms, podcasts, etc., against clinical trials and vaccination, and the interest may be reduced.

Some embodiments may involve ascertaining the reduced interest from the scraped information includes performing semantic analysis on the scraped information. Determining the reduced interest may include using semantic analysis, as described above. For example, the internet may be scraped to find an article about a hospital closing. Semantic analysis may be performed and may find the word “close” in the article. Semantic analysis may determine that the hospital is closing, and no longer interested in an overlapping interest, and therefore the interest may be reduced. As another example, a pattern recognition algorithm may be used to decide, based on historical information, that a hospital, according to a website, may no longer be active, so the interest may be reduced.

Some disclosed embodiments may involve redefining the interest group to exclude the specific one of the selected entities following a determination that the at least one specific overlapping interest of the specific one of the selected entities is reduced. The interest group may be regenerated based on the reduction of the overlapping interest. The interest group may be regenerated so that it does not include the specific entity that is no longer interested in the overlapping interest. Using the example described above where a task of watering vegetables may be assigned to an entity, the interest group may be redefined so that the specific entities that were also interested in watering the vegetables are no longer included in the interest group.

Some disclosed embodiments include determining completion of the common purpose and dissolving the interest group following the determination of completion. Completion of the common purpose may include ascertaining that the common purpose has been achieved. The determination may be made by performing additional semantic analysis or through other means. For example, participating entities may electronically report completion of their tasks. Or, internet scraping combined with semantic analysis may determine that one or more tasks are complete. The interest group may be dissolved by removing the interest group from memory or by marking/flagging it as non-active or deleted. For example, a common purpose may be to perform research on a new drug and write a scientific paper about the drug. Completion of the purpose may occur when the scientific paper is published. The interest group interested in performing research on the drug may be deleted from memory once the paper is published (or markers may be placed in memory to indicate that a common purpose is achieved).

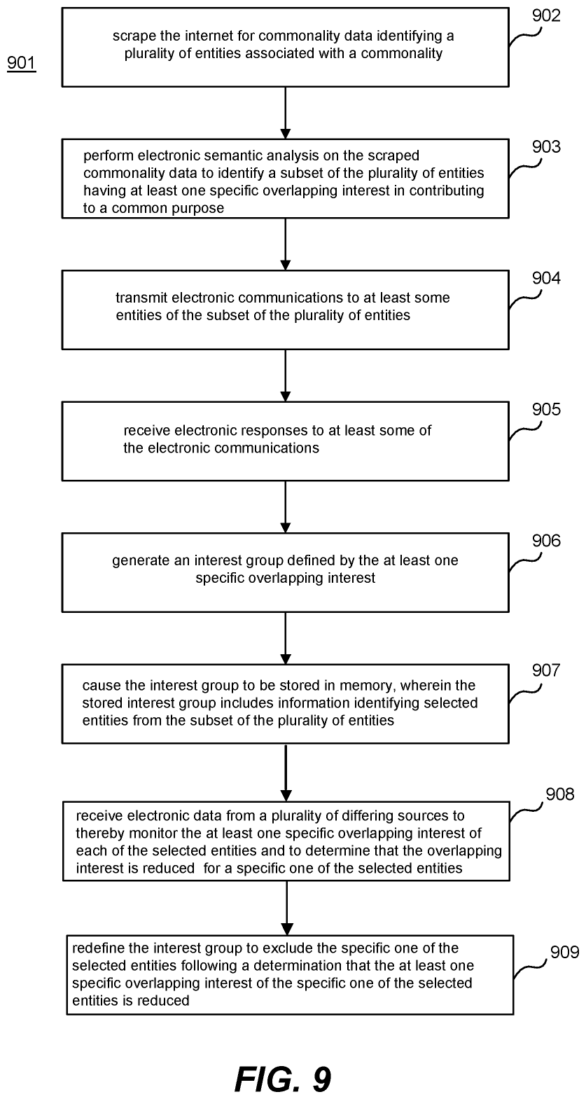

FIG. 9 is a flow diagram of an exemplary process 901 that may be executed by a processor to perform operations for forming ephemeral social clusters, consistent with the disclosed embodiments. In some embodiments, a non-transitory computer readable medium may contain instructions that when executed by a processor cause the processor to perform process 901. Further, process 901 is not necessarily limited to the steps shown in FIG. 9, and any steps or processes of the various embodiments described throughout the present disclosure may also be included in process 901, including those described above with respect to FIG. 9.

Process 901 may include a step 902 of scraping the internet for commonality data identifying a plurality of entities associated with a commonality. For example, this may include various web scraping techniques as described above. Process 901 may also include a step 903 of performing electronic semantic analysis on the scraped commonality data to identify a subset of the plurality of entities having at least one specific overlapping interest in contributing to a common purpose, as described earlier. Further, process 901 may include a step 904 of transmitting electronic communications to at least some entities of the subset of the plurality of entities, consistent with the transmission mechanism described herein.

Process 901 may also include a step 905 of receiving electronic responses to at least some of the electronic communications as discussed previously. Further, process 901 may include a step 906 of generating an interest group defined by the at least one specific overlapping interest. and a step 907 of causing the interest group to be stored in memory, wherein the stored interest group includes information identifying selected entities from the subset of the plurality of entities, as described earlier. Process 901 may also include a step 908 of receiving electronic data from a plurality of differing sources as discussed earlier to thereby monitor the at least one specific overlapping interest of each of the selected entities and to determine that the overlapping interest is reduced for a specific one of the selected entities. Finally, process 901 may include a step 909 of redefining the interest group to exclude the specific one of the selected entities following a determination that the at least one specific overlapping interest of the specific one of the selected entities is reduced, as previously discussed.

As described herein, the disclosed systems may be used to enable entities to work towards resolving an unmet need. In many instances, an unmet technological need remains unmet because complementary skill sets for solving the need do not exist within an organization. Further, organizations often do not realize that needed skills are missing. Accordingly, solutions are needed for automatically identifying individuals associated with skillsets sufficient for resolving an unmet technological need.

Some disclosed embodiments provide solutions for associating entities with unestablished relationships in order to induce collaboration. In particular, such disclosed embodiments may include electronically assessing unmet needs, scraping the internet to identify the types of skill required to solve the need, and recommending a team of previously unconnected entities who have the skills to work together to meet the need. As described herein and illustrated in FIG. 1, a system may include a network of various elements, such as system 100. System 100 may be used to associate entities to induce collaboration. For example, this may include identifying an entity and linking that entity to another entity. As described above, an entity may refer to any distinct or independent existence. For example, an entity may be a person, an organization, an institution, a structure, a device, machine, or any other type of distinct existence. A system may associate entities by forming a connection between the entities. For example, a system may associate a doctor with a hospital. As another example, a system may associate a patient with a doctor. Entities with unestablished relationships may be linked together. An unestablished relationship may indicate the entities have not been associated before. For example, a doctor and a hospital may have an unestablished relationship if the doctor has not previously worked at the hospital. As another example, a patient and a doctor may have an unestablished relationship if the doctor has not seen the patient before. Associating entities with unestablished relationships may induce collaboration. Collaboration may involve entities working together towards a common goal. A system may bring entities together to encourage the entities to form a relationship. For example, a specialist and a local doctor with an unestablished relationship may be associated in order to solve a patient's health need. As another example, an association may be established between plurality of specialists within a technical field to work together to achieve a desired result. For example, in the context of a clinical trial, different specialists with different backgrounds and practical experience may be needed to analyze results and determine next steps. Accordingly, the disclosed embodiments may be implemented to identify these specialists and form a relationship in the context of the clinical trial.

Some disclosed embodiments may involve receiving information identifying a current unmet technological need. As described above, a technological need may refer to a circumstance or set of circumstances that require some course of action that is technical in nature. For example, a technological need may be a need that is related to endeavors that are scientific, biological, engineering-related, industrial, occupational, professional, scholarly, vocational, health-related, medical, chemical, environmental, security-related, mathematical, or otherwise technical in nature. In some embodiments, a technological need may be “unmet” in that one or more aspects of the technological need may still require a course of action. A current unmet technological need may be a need that is relevant to a present endeavor. For example, a current unmet technological need may refer to a hospital's present need for a doctor who specializes in cancer treatment. As another example, an unmet technological need may refer to a patient's present need for a doctor who specializes in heart conditions. The current unmet technological need may be associated with information. This information may include any data that may provide an indication of the current unmet technological need. For example, such information may include, text, images, sounds, audio, videos, or other types of information. The information identifying a current unmet technological need may be received by a client-side computing device, a host computer, a server, a network or any other type of device used to receive information. Such information may be received from a website, a server, a network, a computer, or any other type of device used to send information.

Some disclosed embodiments may involve transmitting at least one query for the current unmet technological need. A query may include a phrase or keyword used to find a thing of interest. For example, a query may be transmitted to computing device 120 requesting that a user 130 provide an indication of a current unmet technological need. In some embodiments, in response to the query, a user may input or select the current unmet technological need through a user interface, such as user interface 430 described above. In some embodiments, the query may be sent out over a network, such as the internet. The transmission over the network may be in the form of signals. In embodiments, where a query is transmitted, the information identifying the current unmet technological need may be received in response to the at least one query. The information received may include information specifically related to the query. For example, the query may include the phrase mentioned above. The information received may include the phone number, physical address, or email address of a dermatologist located near the entity who performed the query.

In some embodiments, receiving information identifying the current unmet technological need may include scraping the internet for the information identifying the current unmet technological need. As described throughout the present disclosure, scraping the internet may include collecting information from websites using various data extraction techniques. For example, the extraction techniques may include full extraction, incremental extraction, structured and unstructured extraction, online and offline extraction, trigger-based extraction, timeline-based extraction, the receipt of data through various APIs, or any other technique through which data may be obtained. The data may then be stored in a central database or location. The data may be stored in CSV format, Excel format, JSON, or any other type of storage means. The internet may be scraped to search for an unmet technological need and for information describing the unmet need. For example, the internet may be scraped to determine that a clinic is searching for participants in a clinical trial. This may be determined by scraping websites such as government websites, job websites, medical websites, or any other type of website. Information relating to the clinical trial may include the dates of the trial, the location of the trial, the type of participant needed, compensation, or any other information related to the trial. In some embodiments, this may include various other types of sources, such as scholarly or professional journals or other publications, or any of the various sources 150 described above. Some embodiments may include scraping the internet to define a historical unmet technological need in a particular situation. For example, through scraping published use cases (e.g., journals, websites, professional forums and directories, etc.) a clinical trial that is related to a specific case of diabetes may be captured. This case may describe an unmet need to measure the size of a wound in each visit to the same standard or protocol.

Some disclosed embodiments involve accessing an electronic data source to identify, in relation to the current unmet technological need, a plurality of skill sets of a plurality of entities, and a plurality of technological need-related roles of the plurality of entities. Such an electronic data source may correlate skill sets and roles with the plurality of entities. For example, the electronic data source may include professional publications, a social platform, published use cases, websites (e.g., Q&A forums, product pages, services, career guides, skill guides, etc.), online books, and more. Information extracted from these sources may be used to define structures of the unmet need. For example, this may include recognizing skill structures in various descriptions, for example, using semantics analysis or various other techniques as described herein. As another example, this may include analyzing user profiles (e.g., LinkedIn profiles, professional database profiles, etc.), people background information, or other personal data to find skills related to a situation. Then, from various other sources described above (such as publications, websites, use cases, etc.), a correlation between the situations and unmet needs may be determined. As another example, a human resources (HR) database with all job interviews may be accessed and analyzed to find patterns and correlations between skills and situations. As another example, this may include analyzing summaries of clinical trials, clinical trials protocols, historical use cases, books, publications, interviews (e.g., through analysis of text, audio, video, etc.) or any source that summarizes the ongoing procedure of a trial. From this information, a description of challenges and how they were overcome may be extracted, along with descriptions of the skills, devices, methods or protocols used in the trial. These factors may be connected to the unmet need and the associated solution.

In some embodiments, the data source may be populated as the result of research, or, for example, internet scraping may identify skill sets associated with particular entities, or skill sets and roles typically associated with a class of entities. Using a data source populated in such a manner or in another manner, the electronic data source may be used to determine skill sets and technological need-related roles associated with the current unmet technological need. An electronic data source may be any source of stored data that may be accessed electronically, including the various examples provided below. The source of the stored data may be a database, file, or any other format. Data may be stored in a table, object, word document, or other storage format within the source. A data source may be accessed by obtaining the data at the location where the data is stored.

In some embodiments, accessing the electronic data source includes accessing a data structure. Data may be stored in a data structure. As described in further detail above, a data structure may be an organization, management, or storage format that allows efficient access and modification of the data. The data structure may be stored locally on a computer, on a network, or through other storage means. For example, this may include accessing database 112, as described in further detail above, or a data structure accessible through network 140. Obtaining data from the electronic data source may include finding data in the data structure and sending the data to a processor.

In some embodiments, accessing the electronic data source may include scraping the internet. Accordingly, the electronic data source may be a source or plurality of sources accessible via the Internet, which may be scraped, as described above, to obtain data from the data source. For example, the skill sets and technological need-related roles may be ascertained from a social network platform (e.g., LinkedIn™, Facebook™, etc.), publications related to the current unmet technological need, or various other Internet-based sources, websites containing descriptions of a product or a service, Q&A websites/products (e.g., Quora™, Stackoverflow™, etc.), professional directories, apps, associations, or organizations (diaglobal.org, acrpnet.org) professional on-line journals (science.org, nejm.org), online use cases, professional publications including scientific publications (e.g., WebMD™, ResearchGate™) government online sources (e.g., clinicaltrials.gov, fda.gov), online jobs or (e.g., Upwork™, Indeed™) eBooks, online Research service providers (e.g., CBinsight™, Gartner™, etc.) and/or any other suitable sources.

The electronic data source may store a plurality of skill sets of a plurality of entities related to the current unmet technological need. A skill set may include an entity's range of skills or abilities (any description about ability to do any kind of job). For example, a surgeon may have a skill set that includes being able to perform surgery. As another example, an ultrasound technician may have a skill set that includes being able to operate imaging equipment, AI-related techniques to serve a specific case. These skills may be determined to be related to the current unmet technological need, as indicated above. For example, if the current unmet technological need is associated with developing a cost-effective treatment for diabetes, skills associated with diabetes research may be identified. In some embodiments, a skill may not necessarily refer to a skill possessed by a person. For example, as described herein, an entity may refer to a broad variety of individuals, groups of individuals, organizations, or things (e.g., machine processes, apps, digital services). For example, an entity may refer to a particular piece of equipment and a skill may refer to a function or effect produced by the equipment. A skill may equally apply to a skill or ability of an organization. A skill may refer to any form of attribute of an entity enabling the entity to perform a particular task or role. For example, a skill may include an availability of an entity, a location of an entity, a desire of the entity to perform a task, an indication of a degree of the abilities of the entity, or the like.

Along with a plurality of skill sets, a plurality of entities may have a plurality of technological need-related roles. Technological need-related roles may refer to the part an entity plays in resolving an unmet technological need. For example, a role associated with counselling skills may be required to address a mental health need, or a role associated with skills for designing a clinical trial may be required to test the efficacy of a new drug or device. The example skill requirements presented herein are provided by way of example, and various other roles may be defined consistent with the disclosed embodiments.

Some disclosed embodiments involve scraping the internet to identify at least one prior solution for resolving a previous unmet technological need, the previous unmet technological need being related to the current unmet technological need, and a plurality of prior roles and a plurality of prior skills associated with the at least one prior solution. The internet may be scraped, as described above, to determine if a prior solution exists for a previous unmet technological need. A previous unmet technological need may be an unmet technological need as described above but may be one that has previously been resolved or addressed. For example, a previous unmet technological need may refer to a hospital's past need for a doctor who specializes in cancer treatment. As another example, a previous unmet technological need may refer to a patient's past need for a doctor who specializes in heart conditions. As another example, a previous unmet technological need may include a need for patients to be included in a cohort for a particular clinical trial related to diabetes. A related, current unmet technological need may be a subsequent need for patients to be included in a cohort, which may be related to diabetes or may be related to another disease or condition. Another example may include the need to measure the healing progress of a wound (or lack thereof), a need to perform a specific test in a non-ideal environment, a need to make sure that patients will get to the visits on time every time. a need to understand incomplete data in a clinical trial activity, in real-time, or various other need. Consistent with the present disclosure, a prior solution may exist for a previous unmet technological need. A prior solution may be a result that satisfies a previous unmet technological need, so the need is no longer unmet. There may be one prior solution or multiple prior solutions that satisfy a previous unmet technological need. For example, a previous unmet technological need may be a hospital's past need for a doctor who specializes in cancer treatment. A prior solution may be a doctor who specializes in cancer treatment working for the hospital. As other examples, a prior solution may include a device that tracks patients' wounds, a mobile lab that could come to patient's home, a special service to drive patients with certain conditions, an AI algorithm designed for clinical trials protocols that recognizes incomplete and send alerts, or the like.

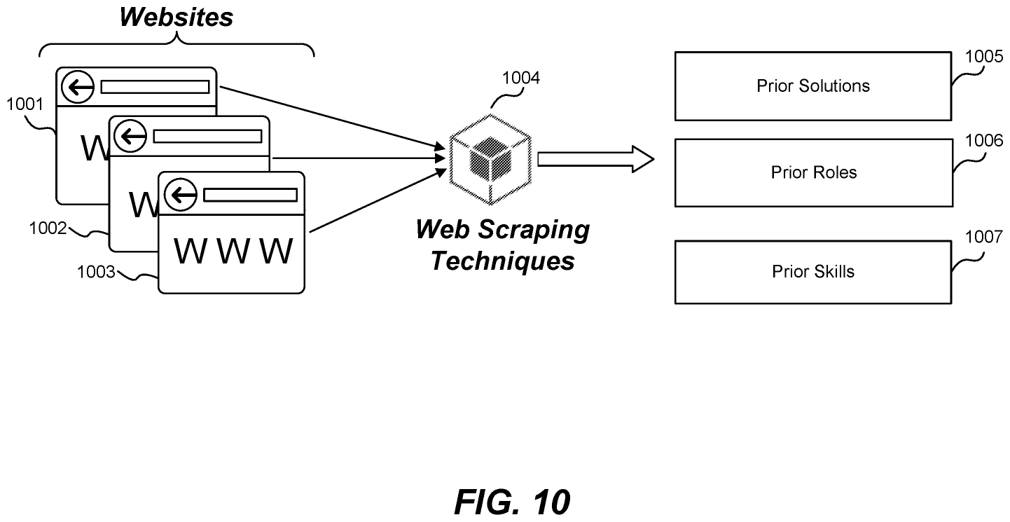

By way of example, FIG. 10 is a diagrammatic representation of an exemplary process for scraping the internet to identify prior solutions, roles, and skills. For example, as illustrated in FIG. 10, websites 1001, 1002, and 1003 may include information. Websites 1001, 1002, and 1003 may correspond to data sources 150, as described above. Web scraping techniques 1004 may include any actions or activities employed to scrape websites 1001, 1002, and 1003 to identify information. For example, web scraping techniques 1004 may include implementing a web crawler and an extraction bot, where the web crawler may be configured to find, index, and/or fetch web pages and documents and the extraction bot may be configured to copy the crawled data and/or process the crawled data, as described above. The scraped information may include prior solutions 1005, prior roles 1006, and prior skills 1007. Consistent with the disclosed embodiments, the prior roles 1006 and the prior skills 1007 may be associated with the prior solutions 1005.

A prior solution may include a plurality of prior roles and a plurality of prior skills A role may include a position or set of responsibilities that an entity held in association with resolving the previously unmet technological need. A prior solution may include a single role or a number of roles. For example, a prior solution may the successful selection of a cohort of patients to participate in a cancer treatment trial and a role may include an oncologist having patients that were selected to be included in the trial. A prior role may include a role that was performed in the past, such as a clinical trial investigator. A prior skill may be the ability to do something, as defined above, and specifically the ability to do something in the past. A prior solution may include a single skill or a number of skills.

In some embodiments, scraping the internet to identify the at least one prior solution, the plurality of prior roles, and the plurality of prior skills may include performing semantic analysis on scraped information. As described herein, semantic analysis may refer to any form of analysis or processing of natural language to draw meaning and/or context. For example, the natural language may be processed in text form. In this context, the semantic analysis may be used to determine that someone having a specialized focus on one or more unmet needs, skills, roles, or expertise. In some embodiments, the scraped information may include at least one of a social platform entry, a blog entry, an article, a news item, website content, or a publication. For example, the internet may be scraped, and the scraped information may include a past job posting for a nurse on a clinical trial page of a hospital's website and a resume that was submitted on the website in response to the job posting. In a first step, for example, relevant sources of information may be identified. For example, the system may collect from a webpage such as ‘clinicaltrials.gov’ (e.g., using an API) all the sites that were running a trial in the past. From websites of these collected sites, the relevant public section with all the job postings may be identified using various techniques. For example, this may include recognizing a job posting template within the data (e.g., based on unique keyword or phrase combinations such as “we are looking for”, “position” “requirements” “responsibilities” and/or using semantic analysis/semantic map for different variations). The template may also be identified using a trained machine learning model. For example, the model may be trained using a plurality of job postings from the past and using pattern recognition process to determine potential template and with user feedback over time to score to determine the best ones. In a second step, the identified sources may be scraped. Semantic analysis may be performed on the scraped information to determine that the resume corresponds to the past job posting. Semantic analysis may analyze the information to determine that the skills identified on the resume. In one non-limiting example, such identified skills might include an ability to draw blood, administer shots, administer an intravenous needle, or other abilities, may be prior skills related to a nursing role. The semantic analysis may determine that the job posting for a nurse may be filled by an entity that may be able to perform those specific skills or may have been in those prior roles.

Some disclosed embodiments involve generating at least one collaboration rule based on the identified prior solution, the identified plurality of prior roles, and the identified plurality of prior skills. As used herein, a collaboration rule may include one or more principles or conditions defining how two or more entities may contribute towards resolving a current unmet technological need. The generated collaboration rule may be an indication of what is needed from two or more entities to resolve an unmet technological need based on the prior solutions, roles, and skills. Accordingly, the past information may be used to create a rule that may be applied to a new situation. The rule may include a condition in which at least two persons or entities may each contribute a skill or an activity that may resolve a new unmet need. At least two persons or entities may partially contribute towards the resolution of a new unmet need in which the two persons or entities may have overlapping interests. For example, a previous unmet technological need may include a need to perform a diagnostic test on a patient with limited mobility (e.g., limited or no access to a vehicle, etc.). The prior solution may include an indication of a lab technician that performed the test, and thus may have skills such as the ability to draw blood or the ability to administer a shot. The solution may also include an indication of a driver who drove a patient to a clinic associated with the lab technician, and the driver's skills may include the ability to transport a patient with limited mobility. In some embodiments, the skills may include other aspects such as the driver's availability, a location of the driver (e.g., near the clinic or patient), etc. The prior roles may therefore include a technician for performing the diagnostic test and a driver. In this example, a collaboration rule may specify that at least one entity with skills and/or roles for performing the test (e.g., a person who has skills such as drawing blood, administering shots, or similar skills) and a driver having skills for transporting the patient (e.g., located near the patient, etc.) are needed to resolve the unmet technological need. The collaboration rule may be defined based on an analysis of scraped data, data structures, existing data of previously resolved unmet needs that may have overlap with the current unmet need, or any other type of data. For example, a collaboration rule may include a rule specifying that a local operator needed to operate a hospital machine be located less than a mile from the hospital. As another example, a collaboration rule may include a rule specifying that a lab technician and a doctor needed for a clinical trial are both able to administer an oral drug. As another example, a collaboration rule may specify a set of skills that the two or more entities have when their skillsets are combined.

In some embodiments, the disclosed methods may involve applying the at least one collaboration rule to the electronic data source to identify, based on the plurality of skill sets and the plurality of technological need-related roles of the plurality of entities, at least two entities of the plurality of entities projected to have an ability to collaborate in order to satisfy the current unmet technological need. In other words, the collaboration rule may be used to find persons or entities that may be able to collaborate to meet an unmet technological need so that the technological need will no longer be unmet. For example, in embodiments where the electronic data source is a data structure, such as a database, this may include performing a lookup function to identify individuals having the identified skills or roles. The persons or entities may possess the skills or technology to be able to work together to fill the unmet technological need. The collaboration rule may be able to determine a person or entity by searching through the electronic data source for specific skill sets or roles that may be previously identified during the formation of the collaboration rule. In embodiments where the electronic data source is accessed through scraping the Internet, identifying the at least two entities may include scraping the Internet to identify the at least two entities. For example, this may include scraping social media platforms, professional directories, professional publications, or various other sources as described above (e.g., Upwork™, Uber™, MTurks™, and many more) to identify entities that have skills or roles similar to the prior skills and/or roles used to achieve the prior solution. Two, three, four, or any suitable number of entities may be identified, which may depend on the particular implementation or unmet technological need.

In some embodiments, the current unmet technological need may be associated with a geographic region and the at least one collaboration rule may specify that the at least two entities be associated with locations within a predetermined range of the geographic region. A geographic region may include a specific area, zone, zip code, land, territory, or other expanse categorized by specific characteristics. A collaboration rule, as defined above, may include a condition that may state that the at least two entities must be in the same location. A location may be a particular place or position. The location may be defined using the geographic region. The location may within a specific distance of the geographic region, within a range of the geographic region, or any other position in relation to the region. A range or distance may be measured in terms of miles, hours, feet, or any other type of measurement. The at least two entities may also be remote and may not located in the same geographical region. For example, a geographic region may be defined as Washington, D.C. The collaboration rule may specify that the at least two entities are within 30 miles of Washington, D.C. As another example, the geographic region may be defined as the area where a hospital is located. The collaboration rule may specify that the at least two entities are within an hour's driving distance from the hospital. Alternatively or additionally, the collaboration rule may not be associated with a particular region and, accordingly, the entities may be located remotely from each other.

In some embodiments, the collaboration rule may include a preference to minimize a quantity of the at least two entities. An optimization process may be used to find the fewest number of entities needed to satisfy the prior roles and the prior skills. For example, if multiple needed skills are possessed by a single entity or spread across a plurality of entities, the rule may opt to select the single entity to thereby minimize a quantity of entities. By way of a more specific example, a prior solution may have been accomplished using five entities, and the system may identify three entities having the same skills that may be able to perform the same roles and accomplish a current unmet technological need. This may provide a more efficient solution as the same result may be achieved with a fewer number of entities.

In some embodiments, the current unmet technological need may be associated with at least a first task and a second task. Accordingly, identifying the least two entities includes identifying a first entity projected to have an ability to perform the first task and a second entity projected to have an ability to perform the second task. A task may include a distinct action that needs to be performed or undertaken to satisfy the unmet technological need. The task may be completed by an entity, such as a person, company, institution, or any other type of thing that may be able to perform the task. Identifying an entity may include discovering an entity that may be able to accomplish the task. The projected ability to perform a task may be determined based on prior experience, education, skills, job roles, or other types on information, which may be similar or the same as the experience, education, skills, or job roles of an entity that successfully completed a task in accordance with the prior solution. A current unmet technological need may be met by performing the task. A current unmet technological need may be met by performing a single task, multiple tasks, or any number of tasks. For example, an unmet technological need may include a need for a testing the efficacy of a new drug. A first task may be the ability to administer the drug. The second task may be the ability to monitor a patient who has been administered the drug. A nurse may be an entity that may be able to perform the first task and a lab technician may be an entity that may be able to perform the second task.

In some embodiments, the current unmet technological need may be associated with a clinical trial and the plurality of prior roles and the plurality of prior skills may be associated with professionals involved in a previous clinical trial. Consistent with the present disclosure, a clinical trial may include a research study used to evaluate a medical, surgical, or behavioral treatment or intervention. In some embodiments, a clinical trial may use human or animal participants to evaluate the efficacy or the treatment. In this example, a current unmet technological need may be related to the clinical trial, such as the need to complete the clinical trial. A professional may be an entity engaged in a specific activity associated with a previous clinical trial. For example, a previous clinical trial may include a trial to test the efficacy of a new oral drug and a professional involved in the previous clinical trial may include a professional to administer the drug. The professional may have a prior skill of being able to select a specific dosage of a drug and may have a prior role as a nurse. The current unmet technological need may be associated with a clinical trial to test the efficacy of a different oral drug. This may include various other professionals, such as physicians having patients that were included in the trial, drivers for transporting patients to a clinic, various different specialists needed to analyze the results of the trial, or any of a wide variety of other entities that may have been involved in the previous clinical trial. In some embodiments, the unmet technological need may correspond to a particular aspect or activity within an overall unmet technological need. Accordingly, the current unmet technological need may refer to a particular activity or aspect of a clinical trial, such as selecting patients for the cohort, administering a treatment, or the like.

In some embodiments, where the current unmet technological need is associated with a clinical trial, the at least one entity of the at least two entities may include a medical facility. As described above, an entity may include a person, place, or thing. A clinical trial may include different types of entities. A clinical trial may include a participant, a medical facility, a doctor, a nurse, or any other type of entity. For example, a clinical trial may include a sleep study. An entity associated with the clinical trial may be a facility to monitor a participant. As another example, a clinical trial may include a medical facility having technicians, equipment, staff availability, or other “skills” needed to perform a particular diagnostic or test.

Some disclosed embodiments involve outputting an identification of the at least two entities in an associative manner in connection with the current unmet technological need. This may include any form or information that identifies the at least two entities along with an indication of the current unmet technological need. An associative manner includes any way of connecting, linking, joining or connecting the entities with the unmet technological need. For example, an identification of the at least two entities may include a telephone number, an email address, a street address, a name, or any other identification means that may be organized or associated with the unmet technological need. An identification may be outputted in an associative manner by providing the identification of at least two entities that shows a way to connect the at least two entities. An output may be the way in which the identification is portrayed. For example, an output may provide an email address for each entity and a template for creating an email to send to both entities. As another example, an output may provide telephone numbers for each entity and a way to create a conference call for contacting both entities. As another example an output may be a video conference link generated and a list of social profiles to share with. Another example may be an online collaboration tool, such as Slack™.

In some embodiments, outputting the identification of the at least two entities in connection with the current unmet technological need may include providing a description of the current unmet technological need. A description of the current unmet technological need may include any information representing or characterizing the unmet need. A description may include words, pictures, objects, text, audio, video, or any other descriptive means. A description may be a way of defining the unmet need. For example, the current unmet technological need may include a need for patients in an underserved community to have surgical intervention in their local clinic. The description may include words such as “surgery”, “doctor”, or may include an image of the clinic.

In some embodiments, outputting the identification of the at least two entities may include outputting an indication of skill sets of the at least two entities. An indication may be a piece of information that shows, characterizes, or reflects a skill set, as defined above. An indication may include text, a position, color, tag, presentation format, order, notation, audible indication, visual indication, or any other types of indication to distinguish a skill set of the at least two entities. For example, a skill set of at least two entities may include the ability to draw blood. The indication may be a picture of a needle with a cartridge containing blood. As another example, a skill set of at least two entities may include the ability to perform surgery. An indication may include a list of the types of surgery the entities may perform.

In some embodiments, outputting the identification of the at least two entities may include outputting an indication of a particular skill possessed by at least one entity of the at least one of the at least two entities. As described above, an identification may include an indication of a skill set. The skill set may be a skill possessed by one entity or by both entities. For example, one entity may possess the ability to draw blood and the other entity may not possess the ability. An indication of a needle with a cartridge containing blood may only appear next to the identification of the entity that possess the ability to draw blood. The indication may be provided in various other forms, such as a text-based description of the particular skill, highlighting on a résumé, curriculum vitale, or profile of an entity, an inclusion or identification of a publication published by the entity, or the like.

In some embodiments, outputting the identification of the at least two entities may include initiating an introduction between the at least two entities. An identification may include a means to connect the at least two entities. An introduction between the at least two entities may include providing a telephone number, email address, physical address, a link to a chat/video chat or another way of contacting an entity to the at least two entities. For example, the identification may output the email address of each entity so that either entity may contact the other entity through email In some embodiments, initiating the introduction may include providing a communication interface for the entities. For example, this may include adding the entities to a chat, a discussion board, or other form of communication-based group so that the entities may communicate with each other.

Some disclosed embodiments may include obtaining additional information regarding the at least two entities prior to initiating the introduction. Additional information may include supplementary information about an entity. The information may include previous work experience, education, technical experience, references, legal issues, or any other type of information. The additional information may be obtained by scraping the internet, as described above. In some embodiments, the additional information may include running a background check on the at least two entities. A background check may include an investigation into a person's previous history, including employment history, education, criminal record, and other activities from a person's past. A background check may be performed by a company or by an individual person.

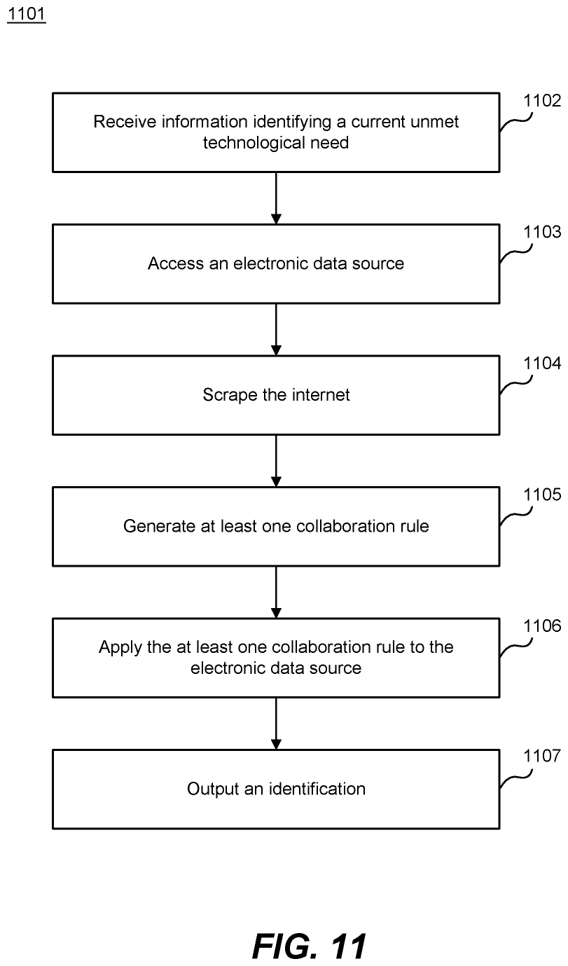

FIG. 11 is a flow diagram of an exemplary process 1101 that may be executed by a processor to perform operations for associating entities with unestablished relationships in order to induce collaboration, consistent with the disclosed embodiments. Process 1101 may be performed by at least one processor, such as processor 210. In some embodiments, a non-transitory computer readable medium may contain instructions that when executed by a processor cause the processor to perform process 1101. Further, process 1101 is not necessarily limited to the steps shown in FIG. 11, and any steps or processes of the various embodiments described throughout the present disclosure may also be included in process 1101, including those described above with respect to FIG. 10.

Process 1101 may include a step 1102 of receiving information identifying a current unmet technological need, as described previously. Process 1101 may also include a step 1103 of accessing an electronic data source to identify, in relation to the current unmet technological need, a plurality of skill sets of a plurality of entities, and a plurality of technological need-related roles of the plurality of entities, as described above. Further, process 1101 may include a step 1104 of scraping the internet to identify at least one prior solution for resolving a previous unmet technological need, the previous unmet technological need being related to the current unmet technological need and a plurality of prior roles, and a plurality of prior skills associated with the at least one prior solution. For example, this may include various web scraping techniques 1004 as described above.

Process 1101 may also include a step 1105 of generating at least one collaboration rule based on the identified prior solution, the identified plurality of prior roles, and the identified plurality of prior skills as described previously. Further, process 1101 may include a step 1106 of applying the at least one collaboration rule to the electronic data source to identify, based on the plurality of skill sets and the plurality of technological need-related roles of the plurality of entities, at least two entities of the plurality of entities projected to have an ability to collaborate in order to satisfy the current unmet technological need, as described previously. Process 1101 may include a step 1107 of outputting an identification of the at least two entities in an associative manner in connection with the current unmet technological need, as described previously.

In some embodiments, prior solutions may be analyzed to address a particular skill gap that has been identified. For example, in the context of a current unmet technological need a skill gap may include the function that key opinion leaders perform. In healthcare, these thought leaders could be physicians, hospital executives, health system directors, researchers, patient advocacy group members, or other professionals, which may act to bridge specialists in certain domains. In the context of clinical trials, different specialists may be needed for analyzing results and determining the next steps, whereas each individual specialist may have a practical experience with only a subset of the problem. The disclosed embodiments may be used to identify a variety of experts who have participated in past trials related to the current one, and then find locally a group of professionals who could together close a skill gap and perform a variety of tasks or roles together to carry out the current trial.

While the various systems and methods for associating entities with unestablished relationships in order to induce collaboration are generally described in association with a clinical trial by way of example, it is to be understood that the disclosed embodiments may be implemented in a wide variety of contexts. The disclosed embodiments may be particularly applicable when prior solutions to an unmet technological need involve the collaboration of entities with a variety of skills and roles. The disclosed embodiments may be implemented to identify a similar group of entities that, when combined, have the same or similar skills and roles.

As another example, an unmet technological need may include the need to address substance abuse within a particular community. A possible conceptual solution could be an intervention new drug or form of group therapy, which may have been implemented previously in other communities. Due to the nature of the need a psychiatrist or clinical psychologist may be required to determine impact of the intervention over time, which may need to occur locally (e.g., face to face). The disclosed embodiments may be implemented to determine the level of expertise and experience within a certain range of the community and, based on the prior solution that has been identified, identify particular psychiatrists or psychologists having skillsets or roles suitable to determine the impact over time. The disclosed techniques may be implemented in association with various other forms of unmet technological needs, including any of the various unmet technological needs described herein.

As described above, some disclosed embodiments may be used to enable entities to work together in order to resolve an unmet need. In some embodiments, this may include identifying two or more unrelated entities to work together to resolve an unmet need. For example, this may include entities from different organizations, remotely located entities, or the like. Many challenges may arise when two or more entities work separately from each other to achieve a common goal. For example, it may be difficult for each entity to monitor the progress of the other entity to ensure they are meeting milestones and contributing as planned. Moreover, even when the goal is reached, achieving the benefits of success may be difficult. For example, based on accomplishing the goal, or completing various milestones toward accomplishing the goal, one or more of the entities may be entitled to a reward or compensation for their contributions. Using conventional techniques, it may be difficult if not impossible to determine when entities are entitled to these contributions efficiently, especially when many entities and many discrete tasks are involved. Accordingly, solutions are needed for monitoring performance according to an agreement, such as a smart contract, and providing recommendations based on various stages of progress.

Some disclosed embodiments provide solutions to addresses these challenges by monitoring compliance with a smart contract, and when the contract requirements are met, identifying individuals or entities that might benefit from the success, and presenting them with an ability to benefit.

Some disclosed embodiments may be implemented to provide internet-based smart contracting and collaboration. As used herein, a smart contract may refer to any form of agreement between two or more parties that is implemented as computer software or code. Smart contracting may include the use of a computer program or a transaction protocol which is intended to automatically execute, control or document legally relevant events and actions according to the terms of a contract or an agreement. In some embodiments, a smart contract may refer to computer code that automatically executes all or parts of an agreement and is stored on a blockchain-based platform. The code can either be the sole manifestation of the agreement between the parties or might complement a traditional text-based contract and execute certain provisions, such as transferring funds from Party A to Party B. If the parties have indicated, such as by initiating a transaction, that certain parameters have been met, the code may execute the step triggered by those parameters. If no such transaction has been initiated, the code may not take any steps. Smart contracting may be desirable to reduce transaction costs, enhance process efficiency, and ensure the security of information. Collaboration includes any form of association, partnership, teamwork, or any other joint work between two or more individuals or entities. For example, collaboration may include one individual working on a project with another individual. As another example, collaboration may include an individual working on a project with a hospital. Internet-based functions may include any actions carried out using the Internet, including sending, receiving, manipulating, or in any other way interacting with any type of information through the Internet. For example, internet-based smart contracting includes the use of computer code that automatically executes all or parts of an agreement and is stored on a blockchain-based platform on the Internet. As another example, internet-based collaboration includes sending and receiving messages through the Internet.

Some disclosed embodiments may involve accessing terms of a collaborative smart contract between at least two previously unconnected entities. A collaborative smart contract may refer to a smart contract that one or more entities can influence or contribute to. For example, a smart contract may include a specifiable time limit for completing a task. In this example, the smart contract may be collaborative by allowing one or more entities to specify the time limit to be four days. As another example, the smart contract may be a contract in which a first entity is responsible for completing some specified tasks and a second entity is responsible for completing other specified tasks. Accordingly, the two entities may collaborate to reach a goal. Terms of a collaborative smart contract include any provision forming part of the collaborative smart contract. For example, the terms may include an acceleration clause, each party's duties, methods of acceptance, an arbitration clause, conditions for performance, consideration, damages, and indemnification. Previously unconnected entities include entities that were not previously in a relationship with one another, such as a business relationship. In some embodiments, previously unconnected entities may not know each other's identities. In other embodiments, previously unconnected entities may know each other's identities. As an example, previously unconnected entities may include a researcher that did not previously have a business relationship with a research clinic. In some embodiments, previously unconnected entities may refer to entities that were not previously introduced or were not aware of each other, entities that work in different organizations (e.g., companies, etc.), entities from different departments, entities that have not yet worked together, or any entities that have not engaged with each other in some form. The unconnected entities may be joined together by contract based on an identified need of one contracting entity and an identified skill, interest, or ability to fulfill that need by another contracting entity. The match can be made by scraping the Internet to identify the entity with the need and the entity with the skill, interest, or ability to fulfill that need. Thus, two entities, who might not know each other and who might never have had opportunity to know each other might be joined together by contract.

In some embodiments, the at least two previously unconnected entities may include a first entity and a second entity respectively located in a first venue and in a second venue. A venue may include a locale, place, setting, site, address, or any other location. For examples, a first entity located in first venue may include a company located in Los Angeles, California, while a second entity located in a second venue may include a company located in Washington, D.C. As another example, a venue may refer to a particular computer-based platform, network, or environment. For example, the first and second venues may refer to different collaboration software platforms and the smart contract may allow the entities to collaborate across different platforms. In some embodiments, the collaborative smart contract may define a plurality of success criteria for resolving an unmet technological need shared by the first entity and the second entity. Success criteria for resolving an unmet technological need may include any standards or levels by which to judge whether an objective, goal, target, or outcome associated with the unmet technological need has been achieved or was sufficiently successful. For example, a technological need may include a need to complete the enrollment for asthma related clinical trial in undeserved community and the need may be unmet in that the clinical trial has not yet been completed. In this example, success criteria for resolving the unmet technological need may include completing a variety of tasks, collecting specified data, and providing the collected data in a specified format.

Some disclosed embodiments may involve scraping the internet to identify the at least two previously unconnected entities. As described throughout the present disclosure, scraping the internet may include an automatic method to obtain data from the internet, such as from websites. Internet scraping may involve various elements, such as a crawler and a scraper. In some embodiments, the crawler may be an artificial intelligence algorithm that browses the internet to search for the particular data required by following the links across the internet. The scraper may extract the data from the internet from a source, such as a website. In some embodiments, the web scraper may be given one or more URLs to load before scraping. The scraper may then load the entire HTML code for the page in question or using APIs. More advanced scrapers may be used to render the entire website, including CSS and Javascript elements. Then the scraper may either extract all the data on the page or specific data selected by a user. Lastly, the web scraper may output all the collected data into a format that is more useful to the user. Examples of the outputted data formats include a CSV or Excel spreadsheet. In some embodiments, more advanced scrapers may be used, which support other formats such as JSON, which can be used for an API. Scraping the internet to identify the at least two previously unconnected entities may be desirable to reduce the processing time of the processor by not forcing the processor to load each webpage associated with the at least two previously unconnected entities. Internet scraping may be used to identify the entities based on any factor associated with each entity, such as a name, location, service offered, specialty, or ranking, skills, abilities, connection to unmet need. For example, internet scraping may be used to identify two previously unconnected software companies based on scraping the internet for websites containing information on companies that provide software services. As another example, internet scraping may be used to identify two previously unconnected software product companies based on scraping the internet for websites containing information on companies with the word “clinical” or “clinical trial screening” in their text. The entities may be scraped from various sources described herein, including professional or scholarly publications, social media platforms, articles, or various other publications or documents. For example, this may include finding services that could join forces with digital services such as a digital legal product (for signing a clinical trial consent agreement) that could be found using keywords such as “econsent”, “digital sign consent” and combined them with a local clinical trial screening services that could be find with keywords such as “clinical trials screening”

In some embodiments, identifying the plurality of entities who share the unmet technological need may further include performing a semantic analysis on scraped information. As described throughout the present disclosure, semantic analysis may involve looking beyond the individual words used on the internet to identify the true meaning of what's being said as a whole. Semantic analysis may be desirable to attach context to internet scraping beyond merely the literal meaning of a word, thereby improving the accuracy of the Internet scraping. In some embodiment, semantic analysis may begin with lexical semantics, which studies individual words' meanings (i.e., dictionary definitions). Semantic analysis may further include examining relationships between individual words and analyzing the meaning of words that come together to form a sentence. This analysis may provide a clear understanding of words in context. In some embodiments, semantic analysis may be performed using machine learning or natural language processing to further improve the accuracy of the internet scraping. For example, identifying the plurality of entities who share the unmet technological need may include identifying a plurality of researchers who share an unmet need of performing certain petri dish testing. If a basic dictionary definition of the term “dish” was used by the internet scraping engine, then entities associated with both petri dishes and dish soap may be identified, resulting in an overloaded processor with unnecessary identifications. However, a semantic analysis engine reduces such processor load by tying the scientific context associated with petri dishes to the term “dish” in order to identify entities associated with only petri dishes. As another example, this may include the ability to understand that different words may have the same meaning in terms of an unmet need. For example, a researcher may mention the same unmet need but use different words such as bacteria testing or describe the need to find if bacteria exist or are present (i.e. in a culture).

Some disclosed embodiments may include generating the terms of the collaborative smart contract and presenting the collaborative smart contract to the at least two previously unconnected entities. The terms may be generated in various ways. In some embodiments, the terms may be selected from a database of possible contract terms, and optimal or preferred terms for these entities may be selected based on information about the entities scraped from the internet. In some embodiments, the terms may be generated using a machine learning model or algorithm from historical data containing records with full contracts (and smart contracts) or any type of relevant agreements or any text describing such process/agreement between any relevant entities. The historical data may be represented in any type of medium and may be from any time frame. For example, a machine learning model may be trained with a set of training contract terms along with information about parties of the smart contract. Accordingly, the model may be trained to generate terms of the collaborative smart contract based on information about the previously unconnected entities. The model may also account for other information, such as information about the venues, information about an unmet technological need, or the like.

It may be desirable for the terms of the collaborative smart contract to be generated automatically to maintain the anonymity of the at least two previously unconnected entities by not requiring them to generate the terms themselves. This may also reduce the burden on the entities to negotiate terms, identify which tasks must be performed, determine who is best suited to perform each task, and determine appropriate rewards or compensation for each entity. Embodiments disclosed herein may leverage vast amounts of data associated with the entities, unmet technological need, previous collaborations (and what led to their successes and failures, etc.) to determine these aspects of the smart contract. It may also be desirable to generate the terms of the collaborative smart contract automatically to improve the accuracy of the terms in the smart contract by limiting human manipulation of the smart contract. In one example, the processor may generate the smart contract terms based on user input, such as by an entity typing in the terms of the smart contract. In another example, the processor may generate the contracts terms using a document template and a controlled natural language (CNL). In this example, the processor may generate the terms through automation by mapping from the document template and the CNL to a formal model that can define the terms and conditions in a contract including temporal constraints and procedures. In another example, the system may scrape the internet and historical data records as described above and extract task phrases that may be connected to conditions/terms and context and then may use a scoring mechanism to determine which of the terms will be the most relevant comparing the context based on plural cases in the past. The formal model may then be translated into an executable smart contract. In this example, contract terms may be generated using a toolchain that generates smart contracts of Hyperledger Fabric from template-based contract documents via a formal model. As another example, an ML model may be applied on a plurality of relevant smart contracts on existing Blockchain networks such as Ethereum, or any other record either online or offline. Smart contracts may be determined to be relevant using semantic analysis. The plurality of smart contracts may be compared using semantic analysis to historical data, as described above, and relevant smart contract templates may be auto generated per situation that may include the same context and/or unmet need and/or entities characteristics. The auto generated relevant smart contracts may include scoring.

Some embodiments disclosed herein may include receiving and maintaining executed forms of the collaborative smart contract from the at least two previously unconnected entities. An executed contract may be received and maintained when sent to a server or other memory device in the form of signals that attest to the accuracy of the executed form. This may include transmitting a form of the contract over a network. For example, server 110 may transmit the terms of the smart contract to computing device 120 over network 140. As another example, the terms of the smart contract may be presented to a user through a user interface, such as an application on computing device 120. The two or more previously unconnected entities may provide an electronic signature or other indication that they agree to be bound to the terms of the contract. In some embodiments, this may include selecting an option to accept the terms in a graphical user interface. Server 110 may receive an indication that the smart contract has been executed. The smart contract may be maintained in various forms. For example, the smart contract may be stored as a document in a storage location, such as database 112. As another example, the smart contract may be implemented as a blockchain-based contract, such that the smart contract is stored in a decentralized manner. It may be desirable to receive and maintain executed forms of the collaborative smart contract to retain a record of an executed contract should the record be needed in the future. Executed forms of the collaborative smart contract include any indication that the smart contract has been executed. As such, executed forms may include any documentation by the at least two previously unconnected entities that the smart contract was executed, such as a signed form. As another example, executed forms may include computer codes that indicate that the smart contract has self-executed based on a completed entity action, such as one or both of the at least two previously unconnected entities completing a specified amount of tests associated with a clinical procedure.

In some embodiments, the collaborative smart contract may be based on a blockchain network, or any type of decentralized network, as indicated above. A blockchain network may include any technical infrastructure that allows applications to access ledger and smart contract services. Instead of a single authority, blockchain relies on a decentralized network of users to validate and record transactions. As such, blockchain transactions are consistent, fast, safe, affordable and tamper-proof, unlike traditional digital transactions. The blockchain, or any type of decentralized network, may be public, private, permissioned or constructed by a group of people, such as a consortium.

In some embodiments, the unmet technological need is associated with a clinical trial and the plurality of success criteria include a plurality of activities for successfully completing the clinical trial. It may be desirable for the unmet technological need to be associated with a clinical trial to improve the accuracy and accelerate the timeline of clinical trials. In some embodiments, at least one activity of the plurality of activities includes activity that is required to conduct clinical trials such as identifying subjects for participation in the clinical trial.

Some disclosed embodiments may involve remotely monitoring, over at least one network, activity of the first entity in the first venue to track progress of the first entity in satisfying a first portion of the success criteria associated with the first entity. Activity of an entity may include any form of behavior, input, interaction, movement, or any other action or state associated with an entity. For example, an activity of an entity may be the entity's completion of a task. As another example, this may include receiving input indicating a task is completed. For example, a blood test result may indicate that a task to perform a blood test is complete. As another example, an activity of an entity may be a change in its location. It may be desirable to remotely monitor such activity to improve data availability. Progress in satisfying a first portion of the success criteria includes any condition associated with the completion of any part of the success criteria. For example, the progress may be completing a first task in a series of tasks. In another example, the progress may be completing a certain percentage of tasks in a set of tasks. For example, the task may include driving the patient to the lab. The progress may be 33% complete after the completion of driving the patient to the lab.

Some disclosed embodiments may involve remotely monitoring, over the at least one network, activity of the second entity in the second venue, to track progress in satisfying a second portion of the success criteria associated with the second entity. It may be desirable to track progress in satisfying a second portion of the success criteria associated with the second entity to reduce redundancy of success criteria completion by assigning different portions of the success criteria to different entities. For example, progress in satisfying a first portion of the success criteria associated with the first entity may include performing clinical testing on men for a vaccine. In this example, progress in satisfying a second portion of the success criteria associated with the second entity may include performing clinical testing on women for the vaccine. In some embodiments, the activity of the entities may be monitored over a network, such as network 140. Accordingly, the progress of the entities may be monitored remotely, which may enable the entities to collaborate more freely in their respective venues.

The activity of the entities may be monitored in various ways. In some embodiments, remotely monitoring the activity of the first entity and the activity of the second entity may include periodically transmitting queries to the first entity and the second entity. A query may include any question or request that is made by a user, computer, or device to another user, computer, or device. In this example, server 110 may periodically transmit queries to the entities, for example, by transmitting queries to computing device(s) 120. The queries may by any form of request for information related to at least a portion of the terms of the smart contract. In some embodiments, a query may be an inquiry as to progress toward completing the contract, whether a milestone triggering an obligation is met, whether a particular task is completed, whether a condition is met, whether a term is complied with, or the like. In some embodiments, the query may be presented to a user via a user interface. For example, the entity may receive a notification indicating a query regarding the smart contract is received. The user may select an option to provide a response, for example, indicating whether the user completed a particular task. In some embodiments, remotely monitoring the activity of the first and second entities may further include receiving responses to the queries and determining the activity of the first and second entities based on the received responses. Remotely monitoring the activity of the first entity and the activity of the second entity by periodically transmitting queries to the first entity and the second entity may be desirable to reduce the load on the processor by not requiring continuous data collection from each entity. For example, remotely monitoring the activity of the first entity and the activity of the second entity may include periodically transmitting a query to the first entity and the second entity regarding whether a test has been completed.

Some embodiments may involve generating the queries. For example, the queries may be generated by at least one processor, such as a processor of server 110. In some embodiments, the queries may be generated based on predefined conditions or based on updated conditions. For example, it may be desirable for the processor to generate the queries in order to optimize the processor functioning by only transmitting queries when required. For example, a query may be generated and transmitted to the first entity and the second entity regarding whether a test has been completed periodically every day. Or, if a contract includes a particular milestone associated with a particular date or a particular duration from execution, some embodiments may generate a query when that particular milestone date is reached. As another example, a query may be generated for one entity based on activity of another entity. For example, a query may be generated and sent probing whether a test has been completed. In yet another example, a query may be generated and transmitted to the first entity and the second entity regarding whether a test has been completed based on additional variables such as user input or any factors associated with clinical testing. In another example, a query may be generated and transmitted to the first entity and the second entity regarding whether a test has been completed using a machine learning engine. As another example, this may include using historical data records to learn about activities that indicate a task completion without being specific about the completion of the task. The historical data records may include different types of information such as activities, machine outputs, and outcomes. For example, receiving a full blood test diagnosis in 100% of the cases may indicate that the patient did the blood test.

In some embodiments, remotely monitoring the activity of the first entity and the activity of the second entity may include scraping the internet to identify publications associated with the first entity and the second entity. A publication may include any book, article, journal, website, blog, piece of music, or other work that is available on the internet. It may be desirable to scrape the internet to identify publications associated with the first entity and the second entity in order to determine whether each entity has completed an activity even if one or both of the entities has not explicitly declared it. Accordingly, embodiments disclosed herein may allow tracking progress of one or more entities with minimal input or involvement by the entities. For example, the activity may include performing antibody testing on 100 subjects. In this example, remotely monitoring the activity of the first entity and the activity of the second entity may include scraping the internet to identify publications associated with the first entity and the second entity that discuss performing antibody testing on 100 subjects. Another example may include monitoring the user's social profile and capturing, for example, the user tagging themself in a clinic as an indication that they arrived at the clinic. Another example may include monitoring a webpage that indicates a change from out-of-stock to in-stock for a product, which may be an indication that they received the product.

In some embodiments, remotely monitoring the activity of the first entity and the activity of the second entity may further include performing a semantic analysis on the publications. Semantic analysis may be performed as described elsewhere in this disclosure. It may be desirable to perform a semantic analysis on the publications in order to determine whether each entity has completed an activity even if one or both of the entities has not explicitly declared it, with adequate context. Published literature or blog posts might contain data indicating that an activity associated with a contract is complete. Semantic analysis may determine this in order to monitor progress of a contract. For example, the activity may include performing antibody testing on 100 subjects. In this example, remotely monitoring the activity of the first entity and the activity of the second entity may include performing a semantic analysis to identify publications discussing the subjects' work and undertaking further semantic analysis to determine that some or all of the 100 tests are complete. The activity of the first entity and the second entity may be monitored in various other ways, such as reporting from the first entity and second entity, receiving feedback or information from a third party, tracking progress indicated in a database or data structure, tracking data recorded in blocks in a blockchain, or any other methods for tracking progress electronically. An example may include analyzing peer review and discussing the conclusion of a specific result as an indication for completing a particular test.

Some disclosed embodiments may further involve determining, based on the remote monitoring of the activity of the first entity, that at least one criterion of the first portion of the success criteria is satisfied. A criterion may include any principle or standard by which something may be judged or decided. For example, success criteria may include different phases of a clinical trial, including phase 1, phase 2, and phase 3. In this example, a criterion of the first portion of the success criteria may include the completion of phase 1. The satisfaction of the success criterion may be determined based on the activity of the first and second entities, as described above. For example, the satisfaction of the success criterion may be determined based on a response to a query or based on scraping the internet. Some embodiments may include causing a reward to be provided to the first entity based on the at least one criterion being satisfied. A reward may include anything provided, allotted, or given to an entity in exchange for satisfying a criterion. For example, a reward may include an amount of money. In another example, a reward may include an award certificate or badge. It may be desirable to cause a reward to be provided to the first entity based on the at least one criterion being satisfied in order to motivate completion of the success criteria. For example, success criteria may include different phases of a clinical trial, including phase 1, phase 2, and phase 3. In this example, a criterion of the first portion of the success criteria may include the completion of phase 1 and the processor may determine that the first entity has completed phase 1. Based on the first entity having completed phase 1, the processor may transmit funds (e.g., in the form of a check, wire transfer, credit to an account associated with the first entity, etc.) for $400 to the first entity.

Some disclosed embodiments may involve determining that all of the success criteria are satisfied. The success criteria may be determined to have been satisfied based at least on the remote monitoring of the activity of the first entity and the activity of the second entity, as described above. For example, this may include comparing the activity of the first entity and second entity to the success criteria to determine whether each of the success criteria have been met. It may be desirable to determine that all of the success criteria are satisfied based on the remote monitoring of the activity of the first entity and the activity of the second entity so that a single entity does not face the burden of completing all of the success criteria or monitoring the progress of multiple unconnected entities to determine whether the success criteria have been met. For example, success criteria may include phase 2 period 1 and phase 2 period 2 testing of a new drug. In this example, disclosed embodiments may be used to remotely monitor a first laboratory to determine whether the first laboratory has completed the phase 2 period 1 testing. The disclosed embodiments may also be used to remotely monitor a second laboratory to determine whether the second laboratory has completed the phase 2 period 2 testing. In this example, the success criteria may be determined to be satisfied when the first laboratory has completed the phase 2 period 1 testing and the second laboratory has completed the phase 2 period 2 testing.

Some disclosed embodiments may involve performing semantic analysis to identify a plurality of additional entities who share the unmet technological need. The processor may perform semantic analysis using techniques similar to those described elsewhere in this disclosure. In some embodiments, the semantic analysis may be performed on data scraped from the internet, as described above. It may be desirable to identify additional entities who share the unmet technological need to aid those entities in satisfying that need. For example, identifying the plurality of entities who share the unmet technological need may include identifying a plurality of researchers who share an unmet need of performing certain petri dish testing. Identifying the plurality of entities through semantic analysis may reduce a processing demand on a system, as described above. For example, if a processor searched a database for such entities using a basic dictionary definition of the term “dish,” then entities associated with both petri dishes and dish soap may be identified, resulting in an overloaded processor with unnecessary identifications. However, a semantic analysis engine reduces such processor load by tying the scientific context associated with petri dishes to the term “dish” in order to identify entities associated with only petri dishes, as described above. As another example, vice presidents of different pharmaceutical companies may have a discussion in a specific forum about their challenges and the need to enroll a diverse population in clinical trials. The system may understand from the title of the forum and/or a topic of a specific discussion that the context may be clinical trial enrollment. and the system may capture from the discussion keywords about “diversity.” For example, terms such as “Hispanic”, “immigrants from Mexico” or “Mexican” may be used to indicate diversity.

Some disclosed embodiments may involve transmitting to the plurality of additional entities an indication that the success criteria are satisfied, and an opportunity related to the satisfied success criteria. Transmitting may include transferring, passing, or otherwise sending information to the plurality of additional entities. The transmission may occur over a wired communication link, such as a cord. The transmission may also occur over a wireless communication link, such as Wi-Fi, Bluetooth™, or a cloud server. An indication may include a visual, audio, or tactile notification, message, symbol, or any other sign that the success criteria are satisfied. For example, the indication may be a notification on a phone screen that all stages of a clinical test are completed. An opportunity may include the ability to contact, collaborate with, communicate with, or otherwise interact with any entity or device related to the satisfied success criteria. It may be desirable to transmit an opportunity related to the satisfied success criteria so that the plurality of additional entities are provided with resources for helping satisfy the unmet technological need. For example, the satisfied unmet technological need may be a completed clinical test on a vaccine for adults. In this example, other entities that may have the same unmet technological need may include vaccine delivery device companies. Thus, the processor may provide an opportunity to such entities related to the completed testing in order to obtain the testing information needed to develop a vaccine delivery device.

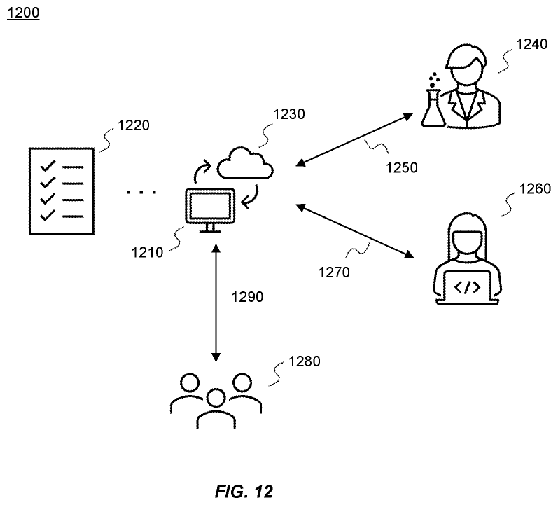

FIG. 12 is a diagrammatic representation of an exemplary system 1200 for internet-based smart contracting and collaboration, consistent with the disclosed embodiments. System 1200 includes a processor 1210 configured to access terms of a collaborative smart contract 1220 between a first entity 1240 and a second entity 1260 respectively located in a first venue and in a second venue. Processor 1210 may be configured to remotely monitor, over network 1230, activity of the first entity 1240 to track progress of the first entity 1240 in satisfying a first portion of the success criteria associated with the first entity 1240 using a communication link 1250, which may be wired or wireless. Processor 1210 may be configured to remotely monitor, over network 1230, activity of the first entity 1240 to track progress of the second entity 1260 in satisfying a second portion of the success criteria associated with the first entity 1260 using a communication link 1270, which may be wired or wireless. Processor 1210 may be configured to, based at least on the remote monitoring of the activity of the first entity 1240 and the activity of the second entity 1260, determine that all of the success criteria are satisfied. Processor 1210 may be configured to perform semantic analysis to identify a plurality of additional entities 1280 who share the unmet technological need. Processor 1210 may communicate with additional entities 1280 using a communication link 1290, which may be wired or wireless. Processor 1210 may be configured to transmit to the plurality of additional entities 1280 an indication that the success criteria are satisfied, and an opportunity related to the satisfied success criteria, using communication link 1290. In some embodiments, system 1200 may correspond to system 100, as described above. For example, processor 1210 may be a processing device associated with server 110, network 1230 may correspond to network 140, and entities 1240, 1260, and 1298 may be associated with various computing devices, such as computing device 120. Accordingly, any of the various descriptions provided herein with respect to system 100 may equally apply to system 1200, and vice versa.

Some disclosed embodiments may further include providing, to at least the first entity and the second entity, an invitation to enter into an additional collaborative smart contract related to the satisfied unmet technological need. An invitation may include any request to participate in an additional collaborative smart contract related to the satisfied unmet technological need. The additional collaborative smart contract may be related to the satisfied unmet technological need based on related or similar characteristics such as time of completion, materials involved, or expertise required. It may be desirable to provide an invitation to enter into an additional collaborative smart contract related to the satisfied unmet technological need in order to match entities with similar goals and capabilities. For example, the satisfied unmet technological need may be a completed clinical test on a vaccine for adults. In this example, it may be desirable to use the same testing methods and conditions when testing the vaccine on children. Therefore, the processor may provide to at least the first entity and the second entity, an invitation to enter into an additional collaborative smart contract to complete clinical testing on the vaccine for children.

In some embodiments, the opportunity related to the satisfied success criteria may include an invitation to contact at least one of the first entity and the second entity. An invitation may include any request to contact at least one of the first entity and the second entity. This may include providing contact information of the first entity or second entity, opening a communication forum such as a chat or messaging platform, initiating a phone call or video conference, or any other means for initiating communication. It may be desirable to provide an invitation to contact at least one of the first entity and the second entity so that other entities with the same unmet technological need may contact entities that satisfied that need to aid in satisfying that need. For example, the satisfied unmet technological need may be a completed clinical test on a vaccine for adults. In this example, other entities that may have the same unmet technological need may include vaccine delivery device companies. Thus, the embodiments disclosed herein may provide an invitation to such entities to contact at least one of the first entity and the second entity in order to obtain the testing information needed to develop a vaccine delivery device.

In some embodiments, the opportunity related to the satisfied success criteria may include an invitation to collaborate with at least one of the first entity and the second entity. An invitation may include any form of request to collaborate with at least one of the first entity and the second entity. The invitation may be provided in similar forms as the invitation to contact the at least one of the first entity and the second entity, as described above. For example, this may include transmitting a message (e.g., an email, text message, etc.), generating an invitation through a social media or professional networking platform, or various other forms of invitations. It may be desirable to provide an invitation to collaborate with at least one of the first entity and the second entity so that other entities with the same unmet technological need may work with entities that satisfied that need to satisfy that need. For example, the satisfied unmet technological need may be a completed clinical test on a vaccine for diverse underserved adults. In this example, other entities that may have the same unmet technological need may include different types of enrollment capabilities of a diverse population. Thus, the processor may provide an invitation to such entities to collaborate with at least one of the first entity and the second entity in order to obtain the testing information needed to develop a vaccine for diverse underserved population.

In some embodiments, the opportunity related to the satisfied success criteria may include an invitation to enter into an additional collaborative smart contract with at least one of the first entity and the second entity. An invitation may include any request to enter into an additional collaborative smart contract with at least one of the first entity and the second entity. It may be desirable to provide an invitation to enter into an additional collaborative smart contract with at least one of the first entity and the second entity so that other entities with the same unmet technological need may work with entities that satisfied that need to satisfy that need. For example, the satisfied unmet technological need may be a completed clinical test on a vaccine for diverse underserved adults. In this example, other entities that may have the same unmet technological need may include different types of enrollment capabilities of a diverse population. Thus, the processor may provide an invitation to such entities to enter into an additional collaborative smart contract with at least one of the first entity and the second entity in order to obtain the testing information needed to develop a vaccine for diverse underserved population.

Some disclosed embodiments may involve generating the terms of the additional smart contract. The terms may be generated using the various techniques described above. In some embodiments, the terms of the additional smart contract may be generated based on user input or the original smart contract. It may be desirable for the processor to generate the terms of the additional smart contract to improve contract accuracy and processor efficiency by limiting redundant contract-generation steps associated with the original smart contract. For example, the satisfied unmet technological need may be a completed clinical test on a vaccine for diverse underserved adults. In this example, other entities that may have the same unmet technological need may include different types of enrollment capabilities of a diverse population. Thus, the terms of an additional collaborative smart contract with at least one of the first entity and the second entity may be generated to include the entity with the enrollment capabilities.

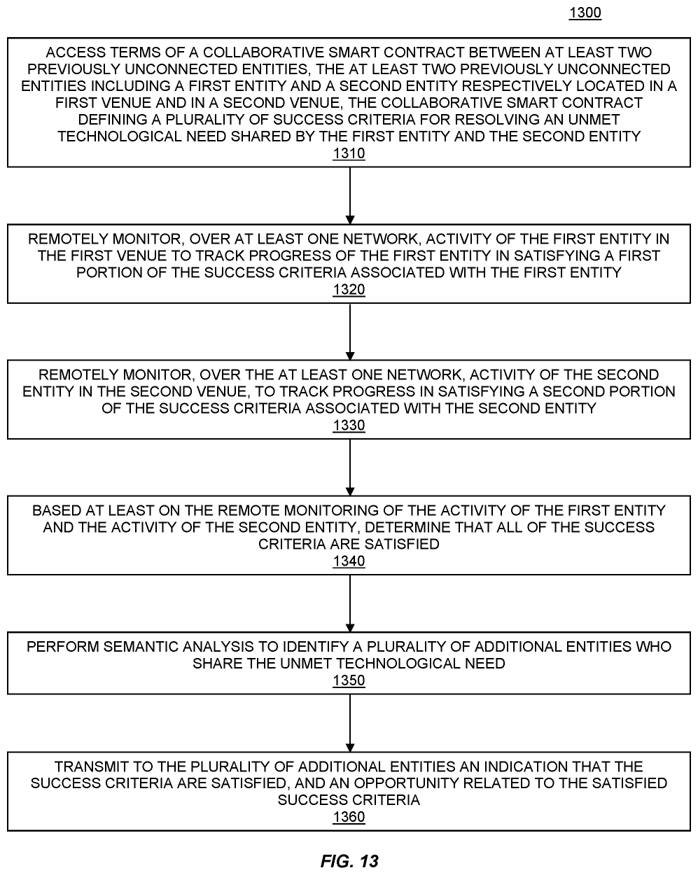

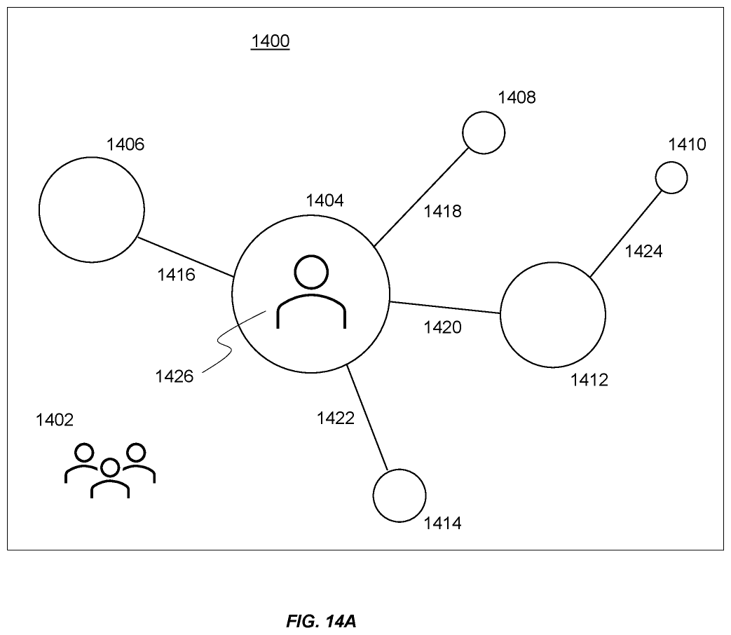

Some disclosed embodiments may involve a computer-implemented method for internet-based smart contracting and collaboration. FIG. 13 is flow diagram of an exemplary method 1300 that may be executed by a computer for internet-based smart contracting and collaboration, consistent with the disclosed embodiments. As shown in step 1310, the method 1300 may involve accessing terms of a collaborative smart contract between at least two previously unconnected entities, the at least two previously unconnected entities including a first entity and a second entity respectively located in a first venue and in a second venue, the collaborative smart contract defining a plurality of success criteria for resolving an unmet technological need shared by the first entity and the second entity. As shown in step 1320, the method 1300 may involve remotely monitoring, over at least one network, activity of the first entity in the first venue to track progress of the first entity in satisfying a first portion of the success criteria associated with the first entity. As shown in step 1330, the method 1300 may involve remotely monitoring, over the at least one network, activity of the second entity in the second venue, to track progress in satisfying a second portion of the success criteria associated with the second entity. As shown in step 1340, the method 1300 may involve based at least on the remote monitoring of the activity of the first entity and the activity of the second entity, determining that all of the success criteria are satisfied. As shown in step 1350, the method 1300 may involve performing sematic analysis to identify a plurality of additional entities who share the unmet technological need. As shown in step 1360, the method 1300 may involve transmitting to the plurality of additional entities an indication that the success criteria are satisfied, and an opportunity related to the satisfied success criteria. The method may offer to create a new smart contract to fulfill the unmet need.

As described herein, some disclosed embodiments may include identifying one or more unmet technological needs for various entities. For example, this may include identifying one or more unmet technological need associated with healthcare providers or other entities. In some embodiments, solutions, resources, or other information collected or generated in association with one unmet technological need may be relevant to another. For example, information associated with one unmet technological need associated with a clinical trial may be relevant to other unmet technological needs associated with clinical trials, even if various aspects of the trial are different. Some disclosed embodiments may include mapping unmet technological needs to identify potential relationships between the unmet technological needs. For example, unmet technological needs may be represented as nodes with connections between the nodes indicating relationships between the unmet technological needs. Various other aspects of the unmet technological needs, including entities, resources, publications, or any other data may also be associated with the nodes. As the needs of various entities change, the map and the connections may also change. Accordingly, some embodiments disclosed herein provide an evolutionary system that ensures that members constantly receive relevant content.

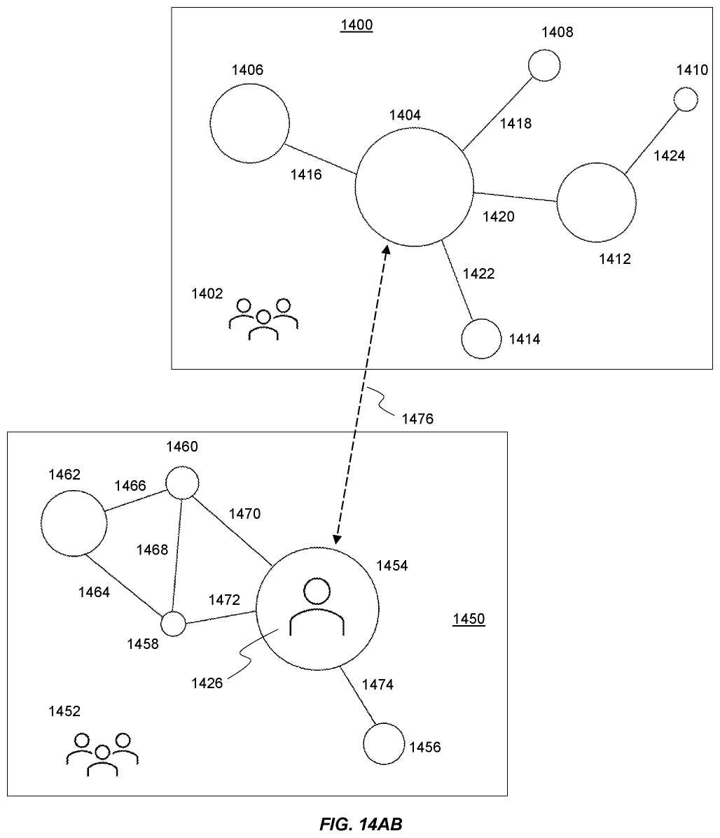

Some disclosed embodiments may be implemented to map a series of related unmet technological needs and to provide resources for satisfying unmet technological needs. As used herein, mapping may refer to the storing of data in a format that is representative of naturally occurring relationships associated with various the elements in the data. For example, mapping may include grouping, linking, relating, aligning, connecting, coupling, any or any other manner of creating a relationship between two or more pieces of information. In some embodiments, the data may be organized into groups. A group may represent a collection of information including one or more pieces of data. These groups may be organized into a network based on relationships in the underlying data. These relationships are referred to herein as mappings. An example of mapping a plurality of items is provided in FIG. 14A. As shown in FIG. 14A, items 1404, 1406, 1408, 1410, 1412, and 1414 may be mapped to one another based on various relationships between one another, as shown by lines 1416, 1418, 1420, 1422, and 1424. For example, items 1404 and 1406 may be linked together because they are related to each other, as shown by line 1416. A series of related unmet technological needs may include a plurality of unmet technological needs that are connected, analogous, complementary, similar, affiliated, correlated, alike, in the same category, interdependent, or otherwise associated with each other in some way. For example, a first unmet technological need may be completing clinical tests on the efficacy of a drug. In this example, a related unmet technological need may include completing the collection of test subjects for the clinical testing. In this example, completing clinical tests on the efficacy of a drug and completing the collection of test subjects for the clinical testing may be related unmet technological needs because each need is associated with the clinical testing. Providing resources for satisfying unmet technological needs may include adding, administering, arranging, bringing, contributing, equipping, furnishing, maintaining, implementing, incorporating, accommodating, procuring, storing, sustaining, or in any other way supplying resources to accomplish, achieve, complete, execute, finalize, or perform any aspect of unmet technological needs. Resources may include abilities, capital, property, systems, equipment, specialties, individuals, organizations, staff, financing, time or schedule availability, or any other type of materials or assets that can be used to perform a function. For example, an unmet technological need may include completing clinical tests on the efficacy of a drug. In this example, providing resources for satisfying this unmet technological need may include providing test subjects, procuring testing equipment, hiring scientists, and securing a laboratory, because these resources can be used to complete the clinical tests on the efficacy of the drug.

Some disclosed embodiments may involve receiving first onboarding information from a plurality of first entities. The first onboarding information may include information indicative of a plurality of first unmet technological needs. Onboarding information may include any information related to the plurality of first entities or the plurality of first unmet technological needs, such as but not limited to: an outstanding task, desired output, education level requirement, resume, location, identities of persons and organizations, costs, and materials to be used for a project. In some embodiments, the onboarding information may be received from an entity during an onboarding process. For example, this may include a process in which an entity registers for a platform or service, generates a user profile, etc. The onboarding information may be received through a user input, such as by clicking or typing, or through data provided by sensors, such as an image capture device, a video capture device, a microphone, a pressure sensor, a temperature sensor, or the like. It may be desirable to receive such onboarding information to organize the mapping of the related unmet technological needs based on their similarities and relationships, as determined from at least the onboarding information. For example, onboarding information may include the educational background of the plurality of first entities, so that the educational background can be used to map the plurality of first unmet technological needs based on the education level associated with each need. A plurality of first entities may include two or more of any entity, such as an individual, organization, company, or association. In some examples, a plurality of first entities may include two or more people. In other examples, a plurality of first entities may include one person and one company. In FIG. 14A, an example of a plurality of first entities 1402 is shown as three individuals.

In some embodiments, the first onboarding information may further include identifications of readiness of the plurality of first entities. Readiness may include a willingness, fitness, preparation, aptness, experience, fluency, inclination, or any other indication of an ability to undertake a task. For example, an unmet technological need may include completing testing of a new software program. In this example, the first onboarding information may include an identification from a first entity that the first entity is willing to complete the software testing. Identifications of readiness may include a description, recognition, classification, or acceptance of readiness. Continuing from the previous example, the first entity may identify their willingness to complete the software testing by clicking on an “I can do this testing” button on a user interface associated with the system. In other instances, the processor may generate identifications of readiness based on stored and collected information. For instance, the processor may collect information on the education level of the plurality of first entities. If the education level matches the level required for completing the software testing in the previous example, the processor may generate an identification of readiness of the plurality of first entities. In some embodiments, the readiness may be determined similar to the availability of an entity, as described herein.

In some embodiments, the first onboarding information may further include identifications of at least one of abilities of the first entities or desires of the first entities. The abilities of the first entities may be identified in a structured manner (e.g., using pre-defined values) or an unstructured manner (e.g., where the user describes their abilities freely through text input). Abilities may also be identified based on external data collected regarding an entity, such as an external profile, past activities, or a website with information on abilities. For example, an ability of the first entities may be the ability to test a specific type of software. The desires of the first may similarly be identified in a structured (pre-defined values) or unstructured (the user describes freely) manner Desires may include an ambition, inclination, need, specialty, or any other want of an entity. For example, a desire of the first entities may be an inclination towards testing software for only mobile applications.

In some embodiments, receiving the first onboarding information may include scraping the internet to identify the plurality of first unmet technological needs and performing a semantic analysis on scraped information. As described herein, scraping the internet may include an automatic method to obtain data from the internet, such as from websites. Scraping to identify the plurality of first unmet technological needs may be desirable to reduce the processing time of a processor by not forcing the processor to load each webpage associated with the plurality of first unmet technological needs. Internet scraping may be used to identify the plurality of first unmet technological needs based on any factor associated with each need, such as a name, location, service offered, specialty, or ranking. For example, identifying an unmet technological need of completing software testing may occur based on scraping the internet for websites containing information on entities that require software testing services.

Semantic analysis may involve looking beyond the individual words used on the internet to identify the true meaning of what's being said as a whole to provide a clear understanding of words in context. In some embodiments, the semantic analysis may be performed using machine learning or natural language processing to further improve the accuracy of the internet scraping. For example, identifying the plurality of first unmet technological needs may include identifying an unmet need of performing certain petri dish testing. If a basic dictionary definition of the term “dish” was used by the internet scraping engine, then needs associated with both petri dishes and dish soap may be identified, resulting in an overloaded processor with unnecessary identifications. However, a semantic analysis engine reduces such processor load by tying the scientific context associated with petri dishes to the term “dish” in order to identify unmet technological needs associated with only petri dishes. As another example, this may include the ability to understand that different words may have the same meaning in terms of an unmet need. For example, a researcher may mention the same unmet need but use different words such as bacteria testing or describe the need to find if bacteria exist or are present (i.e., in a culture).

In some embodiments, receiving the first onboarding information may include querying the plurality of first entities. A query includes any question or request that is made by a user, computer, or device to another user, computer, or device. For example, this may include generating a query by server 110 and transmitting the generated query to another device, such as computing device 120. Receiving the first onboarding information by querying the plurality of first entities may be desirable to reduce the load on the processor by not requiring continuous data collection from each entity. For example, receiving the first onboarding information by querying the plurality of first entities may include periodically transmitting a query to the plurality of first entities regarding whether any software testing needs to be completed. In some embodiments, the querying may be performed in combination with internet scraping or other techniques described herein.

Some disclosed embodiments may further involve identifying similarities between corresponding unmet technological needs by performing a semantic analysis of the first onboarding information. For example, a first unmet technological need may include completing testing on a first software program and a second unmet technological need may include completing testing on a second software program. In this example, identifying the similarities between the first and second unmet technological needs may include performing semantic analysis on needs including the term “tree” to exclude needs that are related to trees in the natural environment rather than software trees. The semantic analysis may also include searching for keywords or phrases that may indicate the similarities. For example, this may include searching for words such as “DevOps,” “build automation,” “source code,” “commit,” or other terms or sets of terms that may indicate the unmet technological needs are both associated with software development. Another example may include finding similarities between unmet needs phrases in order to perform semantic analysis and use ML to find a correlation between phrases based on historical data sets (from use cases, discussions. Q&A, user's direct inputs and selections, etc.).

In some embodiments, the plurality of first unmet technological needs may include unmet medical needs of patients. For example, the similarities between corresponding unmet technological needs may include at least one of a similar disease or a similar condition. Medical needs of patients may include any treatment, test, or procedure that may be necessary to maintain or restore a patient's health. For example, a medical need of a diabetic patient may be insulin. Similar diseases or conditions may include diseases or conditions that are related or alike with regards to their treatment, prognosis, symptoms, or any other characteristic. For example, influenza and allergic rhinitis may have similar symptoms of nose congestion.

In some embodiments, at least one unmet technological need of the plurality of first unmet technological needs includes a regulatory requirement for a clinical trial. Regulatory requirements for a clinical trial include requirements imposed by any private entity or any government regulatory body, such as the Food and Drug Administration. For example, regulatory requirements may include posting of consent forms for federally-funded clinical trials and registration and reporting of clinical trial results.

Some disclosed embodiments may further involve establishing a map of the first unmet technological needs. A map may include any diagrammatic representation of a relationship between two or more pieces of information, such as a, a line graph, or a network diagram. One example of a map is a network diagram, which shows interconnections between a set of data, where each piece of data is represented by a node. For example, the map may include a plurality of nodes representing the first unmet technological needs. The nodes may be connected by connecting structures, such as lines or edges, to show the relationships between the nodes. The established diagrammatic representation of a map may be manifest in a visual manner using visual symbols, or may be manifest within computer code in a non-visual manner FIG. 14A is a diagrammatic representation of an exemplary map of related unmet technological needs, implemented as a network diagram, consistent with some disclosed embodiments. As shown in FIG. 14A, a map 1400 of the first unmet technological needs may be established, including a plurality of nodes 1404, 1406, 1408, 1410, 1412, and 1414 representing the first unmet technological needs. The nodes 1404, 1406, 1408, 1410, 1412, and 1414 are interconnected with lines 1416, 1418, 1420, 1422, and 1424, indicating their relationships with one another. One or more of the shape, color, size, shading, or any other characteristic of the nodes or the lines of a map may be modified to reflect a strength, intensity, value, character, or function associated with an unmet technological need or a relationship between two or more unmet technological needs.

In some embodiments, the map may be associated with a discrete stage of research of a plurality of discrete stages of research. For example, the discrete stage of research may include stages such as an underpinning stage, a conceptual stage, a development stage, a validation stage, a commercialization or scale-up stage, an implementation stage, an adoption and diffusion stage, or any other stages that may be associated with research. It may be desirable to associate the map with a discrete stage of research in order to organize the relationships between the plurality of discrete stages of research. Research may include any systematic investigation into and study of materials and sources in order to establish facts and reach new conclusions, such as quantitative research, qualitative research, mixed research, descriptive research, longitudinal research, cross-sectional research, and action research. For example, the research may be software testing. In this example, the nodes of the map may be associated with the different stages of software testing, such as unit testing, integration testing, system testing, and acceptance testing.

As another example, the discrete stage of research may include a discrete stage of medical research. For example, medical research may include basic medical research, pre-clinical research, and clinical research. In this context, medical research may include any activities for establishing an understanding of the cellular, molecular and physiological mechanisms of human health and disease. Medical research may also include understanding the mechanisms that may lead to clinical research with people. Additionally, medical research may include determining the safety and effectiveness (efficacy) of medications, devices, diagnostic products and treatment regimens intended for human use. For example, the medical research may be clinical research of a new drug. In this example, the discrete stage of research may be Phase II of clinical drug testing.

In some embodiments, establishing the map of the first unmet technological needs may include classifying the first unmet technological needs into a plurality of classifications and wherein each node of the plurality of nodes represents a particular classification of the plurality of classifications. A classification may include an allocation, allotment, analysis, arrangement, coordination, designation, distribution, grade, regulation, assignment, codification, department, or any other categorization of an unmet technological need. For example, the plurality of unmet technological needs may include different phases of clinical drug testing, such as Phase I, Phase II, and Phase III. In this example, each node of the map is associated with one of the phases. In some embodiments, classifying the unmet technological need may include generating or assigning a label associated with the unmet technological need. For example, this may include generating a label such as “clinical trial,” which may be associated with an identified unmet technological need. Accordingly, other unmet technological needs that are related to clinical trials may be assigned different labels. In some embodiments, these labels may be dynamic. For example, two or more unmet technological needs may initially be assigned a label of “clinical trial,” which may initially be a suitable classification. However, if many other unmet technological needs are classified in the same manner, the label may not be particularly useful for distinguishing or classifying unmet technological needs. Accordingly, additional labels or sublabels may be generated, such as a “oncology clinical trial” label and a “diabetes clinical trial” label. In some embodiments, this may similarly include combining or consolidating classifications.

Some disclosed embodiments may involve establishing pathways between the plurality of nodes. As indicated above, the pathways may define relationships between nodes based on similarities between corresponding unmet technological needs. Pathways may connect two or more nodes together. The at least one processor may establish pathways based on similarities in one or more of any characteristics of corresponding unmet technological needs, such as type of activity, education level required, or location. It may be desirable to establish pathways between the plurality of nodes in order to organize the relationships between the plurality of nodes. Examples of pathways are shown in FIG. 14A as lines 1416, 1418, 1420, 1422, and 1424, which define the relationships between nodes 1404, 1406, 1408, 1410, 1412, and 1414.

Some disclosed embodiments may involve associating a particular entity of the plurality of first entities with a first particular node of the plurality of nodes. A particular entity of the plurality of first entities may include any of the plurality of first entities that may be associated with or may aid in satisfying one or more of the first unmet technological needs. For example, in FIG. 14A, a particular entity 1426 of the plurality of first entities 1402 may be associated with a first particular node 1404 of the plurality of nodes 1404, 1406, 1408, 1410, 1412, and 1414. The association may occur in various ways. For example, a representation of entity 1426 may be stored in a data structure, such as a table, array, list, or other data structures herein in an associative manner with node 1404. For example, a data structure may include a plurality of entities and a field associated with entity 1426 may include a reference or indicator of node 1404, indicating that entity 1426 is associated with node 1404. As another example, a separate data structure dedicated to node 1404 may be stored, and entity 1426 may be associated with node 1404 by virtue of being listed in the data structure. One skilled in the art would recognize various other means for storing data in an associative manner, which may be used to form the association.

As an example of the association in practice, a Provider A may provide onboarding information, including an unmet technological need of its patients (e.g., a need to monitor a specific disease for a specific indication), by interacting with a website associated with the system. A funder or Solution-Provider B may then select a button “I can help with this need.” Then, a Provider C may enter the system and describe its patients' unmet technological need. Then, if the system finds that the unmet technological need is similar (e.g., same disease, condition, audience), it could present to Provider C the same funder or Solution-Provider B that selected the “I can help with this need” button in response to the unmet technological need indicated earlier by Provider A. In this example, the plurality of first entities includes A, B, and C, where the particular entity associated with a first particular node of the plurality of nodes is entity B, because entity B is related to both entity A and entity C.

In some embodiments, the particular entity may be associated with the first particular node based on a readiness of the particular entity. The readiness of the entity may be determined as described in further detail above. It may be desirable to associate the particular entity with the first particular node based on a readiness of the particular entity in order to match the particular entity with an unmet technological need based on the interests and background of the particular entity. For example, the first particular node may be associated with completing testing of a new software program. In this example, a particular entity may indicate on a user interface that they are willing to satisfy this need by clicking on an “I can do this testing” button on a user interface associated with the system. The processor then associates this particular entity with the first particular node based on the first entity's readiness, as indicated by clicking on the “I can do this testing” button. As another example, the first onboarding information may further include identifications of at least one of abilities of the first entities or desires of the first entities, as described above. Accordingly, the particular entity is associated with the first particular node based on the at least one of an ability of the particular entity or a desire of the particular entity.

Some disclosed embodiments may involve receiving first content associated with the first particular node. First content may include any information associated with the first particular node, such as information associated with one or more of the first unmet technological needs. In some examples, the first content may be information relevant to satisfying the unmet technological need, such as required materials or personnel needed to complete clinical testing, solutions to an identified unmet technological need, reference materials (e.g., publications, etc.), or any other content items that may be related to an unmet technological need. As another example, the first content may include information that any of the entities entered into the system such as notes, uploaded documents, photographs, videos, 3D models, audio recordings, or any other information. For example, the information may be entered by clicking or typing on a user interface. For example, the particular entity may type in the testing protocol of an unmet technological need associated with completing clinical testing for a new drug. The first content may also include information that the processor determines based on current or historical information introduced to the system. For example, the processor may maintain a record of content associated with a given unmet technological need, such as completing clinical testing for drug A. In this example, the first content may include a testing protocol generated by the processor if the relevant unmet technological need is related to completing clinical testing for drug A. In some embodiments, the first content may be available to the particular entity by virtue of the particular entity being associated with the first particular node. For example, the content may be stored in an associative manner with the first particular node, as described above. For example, this may include storing the content items in association with an identifier of the node (or a plurality of nodes). Accordingly, the content items may be accessible only to entities that are also associated with the node or nodes. In some embodiments, this may include storing the content items in a separate memory or database that is dedicated to the first particular node. Accordingly, only entities associated with the first particular node may be granted access to the memory or database.

In some embodiments, receiving the first content associated with the first particular node may include scraping the internet to determine the first content. Scraping the internet includes an automatic method to obtain data from the internet, as described elsewhere in this disclosure. For example, the relevant unmet technological need may be completing testing on a new software program. In this example, scraping the internet may include automatically extracting data from the internet relevant to software testing websites that describe this sort of testing. In some embodiments, scraping the internet to determine the first content may further include performing semantic analysis on scraped information. For example, the relevant unmet technological need may be completing testing on a new software program. In this example, scraping the internet may include performing semantic analysis on websites including the term “tree” to exclude websites that are related to trees in the natural environment rather than software trees.

As another example, receiving the first content within the first particular node may include accessing information posted by the plurality of first entities. Information posted by the plurality of first entities may include any text, image, video, or any other content published by the plurality of first entities on the Internet. For example, the relevant unmet technological need may be completing testing on a new software program. In this example, receiving the first content within the first particular node may include accessing articles posted by software testing companies. In this context as well as in other contexts, content may be received after identification techniques such as scraping identify the content as relevant. The relevant content may then be accessed and received by a processor.

Some disclosed embodiments may involve receiving second onboarding information from a plurality of second entities, the second onboarding information including information indicative of a plurality of second unmet technological needs. Onboarding information may include any information related to the plurality of second entities or the plurality of second unmet technological needs, such as but not limited to: an outstanding task, desired output, education level requirement, resume, location, identities of persons and organizations, costs, and materials to be used for a project. As described above, the onboarding information may be received through a user input, such as by clicking or typing, or through data provided by sensors, such as pressure or temperature sensors. It may be desirable to receive such onboarding information to organize the mapping of the related unmet technological needs based on their similarities and relationships, as determined from at least the onboarding information. For example, onboarding information may include the educational background of the plurality of second entities, so that the educational background can be used to map the plurality of second unmet technological needs based on the education level associated with each need. A plurality of second entities may include two or more of any entity, such as an individual, organization, company, or association. In some examples, a plurality of second entities may include two or more people. In other examples, a plurality of second entities may include one person and one company. In FIG. 14B, an example of a plurality of second entities 1452 is shown as three individuals.

In some embodiments, the map may be updated dynamically based on the second onboarding information. For example, some disclosed embodiments may involve altering the map based on the second onboarding information. Altering the map may include changing the map in any manner, such as adding or removing nodes or pathways between nodes, or changing the pathways between nodes. In some embodiments, altering the map may include establishing a second particular node of the plurality of second unmet technological needs and establishing a pathway between the first particular node and the second particular node. Altering the map may further include moving the particular entity from the first particular node to the second particular node. For example, the second particular node may be associated with an unmet technological need different from the unmet technological need associated with the first particular node. It may be desirable to alter the map based on the second onboarding information to update how the unmet technological needs and their relationships are organized based on new or relevant information that may be included in the second onboarding information. The second particular node may be associated with an unmet technological need different from the unmet technological need in any way, such as a target, task, personnel, materials, budget, or level of education. The particular entity may be moved from the first particular node to the second particular node for a variety of reasons. In some instances, the second particular node may be indicative of a more relevant unmet technological need of the particular entity. In other instances, the second particular node may be indicative of an additional unmet technological need of the particular entity. Various other relationships between the particular entity and second particular node may be used to determine the map should be altered. For example, machine learning may be used to analyze log files with user activity records and, based on users' selections, alter the map. Another example may include scraping the internet and performing semantic analysis based on the second onboarding information. The semantic analysis may discover that the unmet need is “replacing” the other unmet needs, so the others are not needed anymore. As a further example, there may be a need to improve driving skills in a specific situation. Specifically, an unmet need may be dealing with how to operate an autonomous car in a particular situation. The map may be altered based on information captured from external sources and may determine that in a particular situation only autonomous cars may be used.

FIG. 14B is a diagrammatic representation of an exemplary altered map of related unmet technological needs, consistent with the disclosed embodiments. As shown in FIG. 14B, following the receipt of second onboarding information from the plurality of second entities 1452, map 1450 may be altered to include a second particular node 1454 of the plurality of second unmet technological needs, including other nodes 1456, 1458, 1460, and 1462, which are connected by various pathways, as shown by lines 1464, 1466, 1468, 1470,1472, and 1474. This may include establishing a pathway 1476 between the first particular node 1404 and the second particular node 1454. The particular entity 1426 may be moved from the first particular node 1404 to the second particular node 1454, wherein the second particular node 1454 is associated with an unmet technological need different from the unmet technological need associated with the first particular node 1404.

Some disclosed embodiments may further involve identifying at least one solution associated with the unmet technological need associated with the first particular node and suggesting the at least one solution to an entity associated with the second particular node based on the pathway between the first particular node and the second particular node. A solution may include any manner of satisfying an unmet technological need, such as materials, experience, or budget. It may be desirable to suggest the at least one solution to an entity associated with the second particular node based on the pathway between the first particular node and the second particular node so that similar unmet technological needs may be satisfied using similar solutions, in order to improve efficiency. For example, the first particular node may be associated with completing software testing of a program A, and the second particular node may be associated with completing software testing of a program B. In this example, the processor may identify that a solution for completing software testing of a program A is to hire Person 1 to complete the testing. The processor may then suggest hiring Person 1 to complete the testing to an entity associated with the second particular node based on the pathway between the first particular node and the second particular node.

In some embodiments, identifying the at least one solution may include receiving, from a solution provider, an input indicating that the solution provider can help with the unmet technological need associated with the first particular node. A solution provider may include any entity that is capable of satisfying at least one aspect of an unmet technological need, such as materials, budget, or personnel. For example, in the case of software testing, a solution provider may include a software engineer. The solution provider in this example may also be an HR professional who can staff the testing project with personnel. The input may be received from any input device, such as the I/O devices 270 disclosed herein. For example, in the case of software testing, a software engineer may indicate that the software engineer can help with completing the software testing by clicking on an “I can help with this” button on a website associated with the system.

In some embodiments, identifying the at least one solution may include scraping the internet to identify a solution provider projected to have a solution to the unmet technological need. Scraping the internet includes an automatic method to obtain data from the internet, as described elsewhere in this disclosure. For example, the relevant unmet technological need may include completing testing on a new software program. In this example, scraping the internet may include automatically extracting data from the internet relevant to software testing websites that describe individuals or software solutions that have previously completed the same type of software testing.

Some disclosed embodiments may involve receiving second content within the second particular node, the second content being available to the particular entity by virtue of the particular entity being associated with the second particular node. Once an entity's connection with a node is established, the connection may enable content in that node to be available to the entity. Second content may include any information associated with the second particular node, such as information associated with one or more of the second unmet technological needs. In some examples, the second content may be information relevant to satisfying the unmet technological need, such as required materials or personnel needed to complete clinical testing. As with the first content, the second content may include information that any of the entities entered into the system, such as by clicking or typing on a user interface. For example, the particular entity may type in the testing protocol of an unmet technological need associated with completing clinical testing for a new drug. The second content may also include information that the processor determines based on current or historical information introduced to the system. For example, the processor may maintain a record of content associated with a given unmet technological need, such as completing clinical testing for drug A. In this example, the second content may include a testing protocol generated by the processor if the relevant unmet technological need is related to completing clinical testing for drug A.

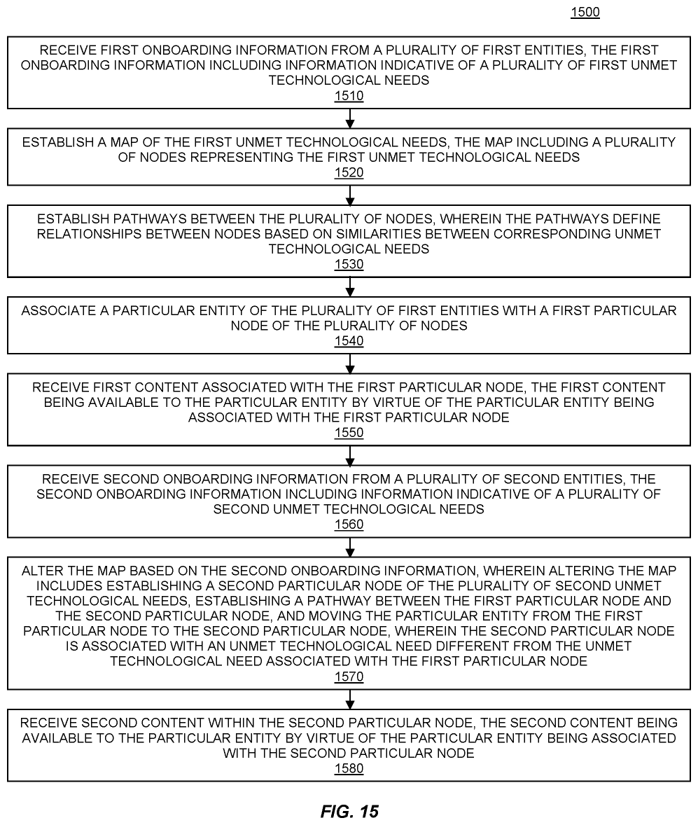

Some disclosed embodiments may involve a computer implemented method for mapping a series of related unmet technological needs. FIG. 15 is flow diagram of an exemplary method 1500 that may be executed by a computer for mapping a series of related unmet technological needs, consistent with the disclosed embodiments. As shown in step 1510, and as described earlier, the method 1500 may involve receiving first onboarding information from a plurality of first entities, the first onboarding information including information indicative of a plurality of first unmet technological needs. As shown in step 1520, the method 1500 may involve establishing a map of the first unmet technological needs, the map including a plurality of nodes representing the first unmet technological needs, as described earlier. As shown in step 1530, the method 1500 may involve establishing pathways between the plurality of nodes, wherein the pathways define relationships between nodes based on similarities between corresponding unmet technological needs, as described earlier. As shown in step 1540 and as also described earlier, the method 1500 may involve associating a particular entity of the plurality of first entities with a first particular node of the plurality of nodes. As shown in step 1550, the method 1500 may involve receiving first content associated with the first particular node, the first content being available to the particular entity by virtue of the particular entity being associated with the first particular node, as described earlier. As previously discussed, and as shown in step 1560, the method 1500 may involve receiving second onboarding information from a plurality of second entities, the second onboarding information including information indicative of a plurality of second unmet technological needs. Additionally, as discussed previously, in step 1570, the method 1500 may involve altering the map based on the second onboarding information, wherein altering the map includes establishing a second particular node of the plurality of second unmet technological needs, establishing a pathway between the first particular node and the second particular node, and moving the particular entity from the first particular node to the second particular node, wherein the second particular node is associated with an unmet technological need different from the unmet technological need associated with the first particular node. Finally, in step 1580, the method 1500 may involve receiving second content within the second particular node, the second content being available to the particular entity by virtue of the particular entity being associated with the second particular node, as described earlier.

As with various other techniques described herein, method 1500 may be applied to a wide variety of industries or uses. One particular use case is the development of “long Covid,” in which patients develop long term conditions resulting from exposure to SARS-CoV-2 or Covid-19. One such aspect is fatigue. In this example, Covid-19 may be a relatively new condition with little available research on aspects such as diagnostics or management of the condition. Instead of exploring all the needs and developments around the disease from scratch, method 1500 may be used to establish connections based on particular conditions (e.g. fatigue). For example, this may include generating a node representing patients suffering from the effects of long Covid. Method 1500 may be employed to find other diseases with chronic fatigue (i.e. due to other viral infection or hormonal imbalance). Accordingly, pathways between the node representing long Covid and nodes representing these other diseases may be established. Accordingly, entities such as patients, physicians, nurses, counselors, family members, etc. may access information advice based on common elements of a condition rather than a disease itself. Another example may include the condition of poor mobility. This condition could be caused by trauma (e.g. fall, traffic accident), diabetic leg ulcers, or various other causes. The need to address poor mobility (e.g. home delivery of meals or transportation services) is related to that condition, not to the underlaying cause or disease. Accordingly, nodes may be connected based on the condition itself—in this case, poor mobility.

Disclosed embodiments may include any one of the following bullet-pointed features alone or in combination with one or more other bullet-pointed features, whether implemented as a method, by at least one processor, and/or stored as executable instructions on non-transitory computer-readable media:
maintaining a data structure containing information about a plurality of unmet technological needs;
receiving a query;
identifying in the data structure a subset of the plurality of unmet technological needs associated with the query;
receiving a selection of a particular unmet technological need from the subset of the plurality of unmet technological needs;
receiving a request to identify an extent of the particular unmet technological need;
scraping the internet to identify a plurality of sources containing information relating to the particular unmet technological need;
performing semantic analysis on the plurality of scraped sources to determine in each scraped source information characterizing an extent of the particular unmet technological need;
aggregating the characterization information from each scraped source;
analyzing the aggregated characterization information to quantify a number of beneficiaries sharing the particular unmet technological need; and
outputting for presentation the quantification in association with an indication of the particular unmet technological need.
wherein receiving the query includes receiving an input from a user through a user interface.
wherein identifying in the data structure a subset of the plurality of unmet technological needs associated with the query includes performing a semantic analysis on the query.
wherein the information characterizing an extent of the particular unmet technological need includes an indication of how close an industry is to resolving the unmet technological need.
wherein the beneficiaries include communities sharing the particular unmet technological need.
wherein the communities include a geographic region affected by the particular unmet technological need.
wherein the operations further comprise analyzing the aggregated characterization information to quantify a degree of impact on the communities sharing the particular unmet technological need.
wherein the unmet technological need is an unmet healthcare need and the degree of impact is associated with a quality of life of individuals in the communities. wherein the beneficiaries include individuals sharing the particular unmet technological need.
wherein the individuals include patients suffering from a medical condition associated with the particular unmet technological need.
wherein the operations further comprise analyzing the aggregated characterization information to quantify a degree of impact on the individuals sharing the particular unmet technological need.
wherein the particular unmet technological need is an unmet healthcare need and the degree of impact is associated with a quality of life of the individuals.
wherein the operations further comprise analyzing the aggregated characterization information to quantify an economic impact associated with the unmet technological need.
wherein the economic impact includes an economic impact on communities sharing the particular unmet technological need.
wherein the economic impact includes an economic impact on an industry associated with the particular unmet technological need.
receiving an indication of an unmet technological need of at least one entity;
electronically extracting data from a data source to identify a plurality of requirements for fulfilling the unmet technological need;
performing a first scraping of the internet to identify at least one solution satisfying at least one of the plurality of requirements;
performing a second scraping of the internet to identify at least one of: a proof of concept for the identified at least one solution; a degree of safety associated with the identified at least one solution; an economic feasibility associated with the identified at least one solution; and a commercial applicability associated with the identified at least one solution; and
analyzing the identified at least one solution, and the at least one of the scraped proof of concept, the scraped degree of safety, the scraped economic feasibility, or the scraped commercial applicability to thereby recommend implementation of at least one specific solution from the identified at least one solution.
wherein the at least one solution includes a plurality of solutions.
wherein the operations further comprise ranking the plurality of solutions based on at least one of the proof of concept, the degree of safety, the economic feasibility, or the commercial applicability.
wherein recommending implementation of the at least one specific solution includes selecting the at least one particular solution based on the ranking
wherein the at least one proof of concept includes information indicating how the identified at least one solution can be executed.
wherein the degree of safety includes an indication of potential harm that may arise from implementing the identified at least one solution.
wherein the economic feasibility includes a cost/benefit analysis for implementing the identified at least one solution.
wherein the commercial applicability includes an indication of how effectively the identified at least one solution can be implemented in a particular industry.
wherein the first scraping includes performing semantic analysis on scraped information.
wherein performing the second scraping includes performing semantic analysis on scraped information.
wherein performing the second scraping includes scraping a plurality of media items.
wherein the recommended implementation of at least one specific solution is further based on additional information including at least one of an indication of available resources or an expertise of a querying individual.
wherein the additional information is accessed from a database.
wherein the unmet technological need includes a resource needed for conducting a clinical trial.
wherein the resource needed for conducting the clinical trial is identified based on a least one trial protocol parameter.
wherein the resource needed for conducting the clinical trial is identified based on application of a trained machine learning model to the least one trial protocol parameter.
wherein the resource needed for conducting the clinical trial includes a specified piece of equipment and wherein the at least one specific solution includes an identification of at least one of a supplier, an operator, or a funding source for the specified piece of equipment.
wherein the resource needed for conducting the clinical trial includes an individual with a specified skillset and wherein the at least one specific solution includes an identification of at least one of the individuals having the specified skillset, a method for training an individual to obtain the specified skillset, or an individual qualified to train an individual to obtain the specified skillset.
scraping the internet for commonality data identifying a plurality of entities associated with a commonality
performing electronic semantic analysis on the scraped commonality data to identify a subset of the plurality of entities having at least one specific overlapping interest in contributing to a common purpose
transmitting electronic communications to at least some entities of the subset of the plurality of entities
receiving electronic responses to at least some of the electronic communications based on the received responses, generating an interest group defined by the at least one specific overlapping interest
causing the interest group to be stored in memory, wherein the stored interest group includes information identifying selected entities from the subset of the plurality of entities
receiving electronic data from a plurality of differing sources to thereby monitor the at least one specific overlapping interest of each of the selected entities and to determine that the overlapping interest is reduced for a specific one of the selected entities redefining the interest group to exclude the specific one of the selected entities following a determination that the at least one specific overlapping interest of the specific one of the selected entities is reduced
wherein receiving electronic data from a plurality of differing sources includes scraping the internet for information about the selected entities and ascertaining reduced interest from the scraped information
wherein ascertaining the reduced interest from the scraped information includes performing semantic analysis on the scraped information
wherein the electronic data from a plurality of differing sources includes electronic data stored in a data structure
wherein the information stored in the data structure includes status information wherein receiving electronic data from a plurality of differing sources includes scraping local data structures
wherein performing electronic semantic analysis on the scraped commonality data includes performing initial semantic analysis to identify the subset of the plurality of entities, and wherein the at least one processor is further configured to perform subsequent semantic analysis to identify potentially available entities within the subset of the plurality of entities
wherein the processor is further configured to subsequently scrape the internet to identify at least one new entity for inclusion within the subset of the plurality of entities
wherein the at least one processor is further configured to send an additional electronic communication to the at least one new entity and, based on a response to the additional electronic communication, include the at least one new entity in the interest group
wherein the at least one processor is further configured to determine completion of the common purpose and to dissolve the interest group following the determination of completion
wherein with the plurality of entities are subscribers to at least one platform
wherein the determination that the at least one specific overlapping interest is reduced by the specific one of the selected entities is based on a determination by the at least one processor that the specific one of the selected entities completed a role associated with the common purpose
wherein the determination that the at least one specific overlapping interest is reduced by the specific one of the selected entities is based on a determination by the at least one processor that the specific one of the selected entities failed to timely complete a task associated with the common purpose
wherein the at least one processor is further configured to suggest an action to the at least one selected entity
wherein the suggested action includes executing at least one of an electronic nondisclosure agreement or an electronic engagement contract
wherein the suggested action includes generating terms of a smart contract
wherein the electronic communications include an invitation to contribute to the common purpose
wherein the common purpose includes conducting a clinical trial and wherein each of the selected entities are associated with at least one skill for conducting the clinical trial
receiving information identifying a current unmet technological need
accessing an electronic data source to identify, in relation to the current unmet technological need, a plurality of skill sets of a plurality of entities, and a plurality of technological need-related roles of the plurality of entities
scraping the internet to identify at least one prior solution for resolving a previous unmet technological need, the previous unmet technological need being related to the current unmet technological need; and a plurality of prior roles and a plurality of prior skills associated with the at least one prior solution
generating at least one collaboration rule based on the identified prior solution, the identified plurality of prior roles, and the identified plurality of prior skills
applying the at least one collaboration rule to the electronic data source to identify, based on the plurality of skill sets and the plurality of technological need-related roles of the plurality of entities, at least two entities of the plurality of entities projected to have an ability to collaborate in order to satisfy the current unmet technological need
outputting an identification of the at least two entities in an associative manner in connection with the current unmet technological need
wherein receiving information identifying the current unmet technological need includes scraping the internet for the information identifying the current unmet technological need
wherein the at least one processor is further configured to transmit at least one query for the current unmet technological need, and wherein the information identifying the current unmet technological need is received in response to the at least one query
wherein accessing the electronic data source includes accessing a data structure
wherein accessing the electronic data source includes scraping the internet
wherein outputting the identification of the at least two entities in connection with the current unmet technological need includes providing a description of the current unmet technological need
wherein outputting the identification of the at least two entities includes outputting an indication of skill sets of the at least two entities
wherein outputting the identification of the at least two entities includes outputting an indication of a particular skill possessed by at least one entity of the at least one of the at least two entities
wherein outputting the identification of the at least two entities includes initiating an introduction between the at least two entities
wherein the at least one processor is further configured to obtain additional information regarding the at least two entities prior to initiating the introduction
wherein obtaining the additional information includes running a background check on the at least two entities
wherein scraping the internet to identify the at least one prior solution, the plurality of prior roles, and the plurality of prior skills includes performing semantic analysis on scraped information
wherein the scraped information includes at least one of a social platform entry, a blog entry, an article, a news item, website content, or a publication
wherein the current unmet technological need is associated with at least a first task and a second task, and wherein identifying the least two entities includes identifying a first entity projected to have an ability to perform the first task and a second entity projected to have an ability to perform the second task
wherein the current unmet technological need is associated with a clinical trial and
wherein the plurality of prior roles and the plurality of prior skills are associated with professionals involved in a previous clinical trial
wherein the current unmet technological need is associated with a geographic region and
wherein the at least one collaboration rule specifies that the at least two entities be associated with locations within a predetermined range of the geographic region
wherein the current unmet technological need is associated with a clinical trial and the at least one entity of the at least two entities includes a medical facility
wherein the collaboration rule includes a preference to minimize a quantity of the at least two entities
accessing terms of a collaborative smart contract between at least two previously unconnected entities, the at least two previously unconnected entities including a first entity and a second entity respectively located in a first venue and in a second venue, the collaborative smart contract defining a plurality of success criteria for resolving an unmet technological need shared by the first entity and the second entity
remotely monitoring, over at least one network, activity of the first entity in the first venue to track progress of the first entity in satisfying a first portion of the success criteria associated with the first entity
remotely monitoring, over the at least one network, activity of the second entity in the second venue, to track progress in satisfying a second portion of the success criteria associated with the second entity
based at least on the remote monitoring of the activity of the first entity and the activity of the second entity, determining that all of the success criteria are satisfied
performing semantic analysis to identify a plurality of additional entities who share the unmet technological need
transmitting to the plurality of additional entities an indication that the success criteria are satisfied, and an opportunity related to the satisfied success criteria
wherein the at least one processor is further configured to scrape the internet to identify the at least two previously unconnected entities
wherein identifying the plurality of entities who share the unmet technological need further includes performing a semantic analysis on scraped information
wherein the at least one processor is configured to generate the terms of the collaborative smart contract and present the collaborative smart contract to the at least two previously unconnected entities
wherein the at least one processor is configured to receive and maintain executed forms of the collaborative smart contract from the at least two previously unconnected entities
wherein remotely monitoring the activity of the first entity and the activity of the second entity includes periodically transmitting queries to the first entity and the second entity
wherein the at least one processor is configured to generate the queries
wherein the at least one processor is further configured to provide, to at least the first entity and the second entity, an invitation to enter into an additional collaborative smart contract related to the satisfied unmet technological need
wherein the opportunity related to the satisfied success criteria includes an invitation to contact at least one of the first entity and the second entity
wherein the opportunity related to the satisfied success criteria includes an invitation to collaborate with at least one of the first entity and the second entity
wherein the opportunity related to the satisfied success criteria includes an invitation to enter into an additional collaborative smart contract with at least one of the first entity and the second entity
wherein the at least one processor is further configured to generate the terms of the additional smart contract
wherein remotely monitoring the activity of the first entity and the activity of the second entity includes scraping the internet to identify publications associated with the first entity and the second entity
wherein remotely monitoring the activity of the first entity and the activity of the second entity further includes performing a semantic analysis on the publications
wherein the collaborative smart contract is based on a blockchain network
wherein the unmet technological need is associated with a clinical trial and wherein the plurality of success criteria include a plurality of activities for successfully completing the clinical trial
wherein at least one activity of the plurality of activities includes identifying subjects for participation in the clinical trial
determining, based on the remote monitoring of the activity of the first entity, that at least one criterion of the first portion of the success criteria is satisfied
causing a reward to be provided to the first entity based on the at least one criterion being satisfied
receiving first onboarding information from a plurality of first entities, the first onboarding information including information indicative of a plurality of first unmet technological needs
establishing a map of the first unmet technological needs, the map including a plurality of nodes representing the first unmet technological needs
establishing pathways between the plurality of nodes, wherein the pathways define relationships between nodes based on similarities between corresponding unmet technological needs
associating a particular entity of the plurality of first entities with a first particular node of the plurality of nodes
receiving first content associated with the first particular node, the first content being available to the particular entity by virtue of the particular entity being associated with the first particular node
receiving second onboarding information from a plurality of second entities, the second onboarding information including information indicative of a plurality of second unmet technological needs
altering the map based on the second onboarding information, wherein altering the map includes establishing a second particular node of the plurality of second unmet technological needs, establishing a pathway between the first particular node and the second particular node, and moving the particular entity from the first particular node to the second particular node, wherein the second particular node is associated with an unmet technological need different from the unmet technological need associated with the first particular node
receiving second content within the second particular node, the second content being available to the particular entity by virtue of the particular entity being associated with the second particular node
wherein the first onboarding information further includes identifications of readiness of the plurality of first entities
wherein the particular entity is associated with the first particular node based on a readiness of the particular entity
wherein the first onboarding information further includes identifications of at least one of abilities of the first entities or desires of the first entities and wherein the particular entity is associated with the first particular node based on the at least one of an ability of the particular entity or a desire of the particular entity
wherein receiving the first onboarding information includes scraping the internet to identify the plurality of first unmet technological needs and performing a semantic analysis on scraped information
wherein receiving the first onboarding information includes querying the plurality of first entities
wherein receiving the first content associated with the first particular node includes scraping the internet to determine the first content
wherein scraping the internet to determine the first content includes performing semantic analysis on scraped information
wherein receiving the first content within the first particular node includes accessing information posted by the plurality of first entities
wherein the map is associated with a discrete stage of research of a plurality of discrete stages of research
wherein the discrete stage of research is a discrete stage of medical research
wherein establishing the map of the first unmet technological needs includes classifying the first unmet technological needs into a plurality of classifications and wherein each node of the plurality of nodes represents a particular classification of the plurality of classifications
identifying at least one solution associated with the unmet technological need associated with the first particular node
suggesting the at least one solution to an entity associated with the second particular node based on the pathway between the first particular node and the second particular node
wherein identifying the at least one solution includes receiving, from a solution provider, an input indicating that the solution provider can help with the unmet technological need associated with the first particular node
wherein identifying the at least one solution includes scraping the internet to identify a solution provider projected to have a solution to the unmet technological need
wherein the at least one processor is further configured to identify the similarities between corresponding unmet technological needs by performing a semantic analysis of the first onboarding information
wherein the plurality of first unmet technological needs includes unmet medical needs of patients and wherein the similarities between corresponding unmet technological needs include at least one of a similar disease or a similar condition
wherein at least one unmet technological need of the plurality of first unmet technological needs includes a regulatory requirement for a clinical trial

Further features of embodiments are set out in the following clauses:
1. A non-transitory computer readable medium including instructions that, when executed by at least one processor, cause the at least one processor to perform operations for identifying unmet technological needs, the operations comprising:
   maintaining a data structure containing information about a plurality of unmet technological needs;
   receiving a query;
   identifying in the data structure a subset of the plurality of unmet technological needs associated with the query;
   receiving a selection of a particular unmet technological need from the subset of the plurality of unmet technological needs;
   receiving a request to identify an extent of the particular unmet technological need;
   scraping the internet to identify a plurality of sources containing information relating to the particular unmet technological need;
   performing semantic analysis on the plurality of scraped sources to determine in each scraped source information characterizing an extent of the particular unmet technological need;
   aggregating the characterization information from each scraped source;
   analyzing the aggregated characterization information to quantify a number of beneficiaries sharing the particular unmet technological need; and
   outputting for presentation the quantification in association with an indication of the particular unmet technological need.
2. The non-transitory computer readable medium of clause 1, wherein receiving the query includes receiving an input from a user through a user interface.
3. The non-transitory computer readable medium of clause 1, wherein identifying in the data structure a subset of the plurality of unmet technological needs associated with the query includes performing a semantic analysis on the query.
4. The non-transitory computer readable medium of clause 1, wherein the information characterizing an extent of the particular unmet technological need includes an indication of how close an industry is to resolving the unmet technological need.
5. The non-transitory computer readable medium of clause 1, wherein the beneficiaries include communities sharing the particular unmet technological need.
6. The non-transitory computer readable medium of clause 5, wherein the communities include a geographic region affected by the particular unmet technological need.
7. The non-transitory computer readable medium of clause 5, wherein the operations further comprise analyzing the aggregated characterization information to quantify a degree of impact on the communities sharing the particular unmet technological need.
8. The non-transitory computer readable medium of clause 7, wherein the unmet technological need is an unmet healthcare need and the degree of impact is associated with a quality of life of individuals in the communities.
9. The non-transitory computer readable medium of clause 1, wherein the beneficiaries include individuals sharing the particular unmet technological need.
10. The non-transitory computer readable medium of clause 9, wherein the individuals include patients suffering from a medical condition associated with the particular unmet technological need.
11. The non-transitory computer readable medium of clause 9, wherein the operations further comprise analyzing the aggregated characterization information to quantify a degree of impact on the individuals sharing the particular unmet technological need.
12. The non-transitory computer readable medium of clause 11, wherein the particular unmet technological need is an unmet healthcare need and the degree of impact is associated with a quality of life of the individuals.
13. The non-transitory computer readable medium of clause 1, wherein the operations further comprise analyzing the aggregated characterization information to quantify an economic impact associated with the unmet technological need.
14. The non-transitory computer readable medium of clause 13, wherein the economic impact includes an economic impact on communities sharing the particular unmet technological need.
15. The non-transitory computer readable medium of clause 13, wherein the economic impact includes an economic impact on an industry associated with the particular unmet technological need.
16. A system for identifying unmet technological needs, the system comprising:
   at least one processor configured to:
      maintain a data structure containing information about a plurality of unmet technological needs;
      receive a query;
      identify in the data structure a subset of the plurality of unmet technological needs associated with the query;
      receive a selection of a particular unmet technological need from the subset of the plurality of unmet technological needs;
      receive a request to identify an extent of the particular unmet technological need;
      scrape the internet to identify a plurality of sources containing information relating to the particular unmet technological need;
      perform semantic analysis on the plurality of scraped sources to determine in each scraped source information characterizing an extent of the particular unmet technological need;
      aggregate the characterization information from each scraped source;
      analyze the aggregated characterization information to quantify a number of beneficiaries sharing the particular unmet technological need; and
      output for presentation the quantification in association with an indication of the particular unmet technological need.
17. The system of clause 16, wherein identifying in the data structure a subset of the plurality of unmet technological needs associated with the query includes performing a semantic analysis on the query.
18. The system of clause 16, wherein the information characterizing an extent of the particular unmet technological need includes an indication of how close an industry is to resolving the unmet technological need.
19. The system of clause 16, wherein the operations further comprise analyzing the aggregated characterization information to quantify an economic impact associated with the unmet technological need.
20. A computer-implemented method for identifying unmet technological needs, the method comprising:
   maintaining a data structure containing information about a plurality of unmet technological needs;
   receiving a query;
   identifying in the data structure a subset of the plurality of unmet technological needs associated with the query;
   receiving a selection of a particular unmet technological need from the subset of the plurality of unmet technological needs;
   receiving a request to identify an extent of the particular unmet technological need;
   scraping the internet to identify a plurality of sources containing information relating to the particular unmet technological need;
   performing semantic analysis on the plurality of scraped sources to determine in each scraped source information characterizing an extent of the particular unmet technological need;
   aggregating the characterization information from each scraped source;
   analyzing the aggregated characterization information to quantify a number of beneficiaries sharing the particular unmet technological need; and
   outputting for presentation the quantification in association with an indication of the particular unmet technological need.
21. A non-transitory computer readable medium for identifying solutions to unmet technological needs, the computer readable medium containing instructions that when executed by at least one processor, cause the at least one processor to perform operations comprising:
   receiving an indication of an unmet technological need of at least one entity;
   electronically extracting data from a data source to identify a plurality of requirements for fulfilling the unmet technological need;
   performing a first scraping of the internet to identify at least one solution satisfying at least one of the plurality of requirements;
   performing a second scraping of the internet to identify at least one of:
      a proof of concept for the identified at least one solution;
      a degree of safety associated with the identified at least one solution;
      an economic feasibility associated with the identified at least one solution; and
      a commercial applicability associated with the identified at least one solution; and
   analyzing the identified at least one solution, and the at least one of the scraped proof of concept, the scraped degree of safety, the scraped economic feasibility, or the scraped commercial applicability to thereby recommend implementation of at least one specific solution from the identified at least one solution.
22. The non-transitory computer readable medium of clause 21, wherein the at least one solution includes a plurality of solutions.
23. The non-transitory computer readable medium of clause 22, wherein the operations further comprise ranking the plurality of solutions based on at least one of the proof of concept, the degree of safety, the economic feasibility, or the commercial applicability.
24. The non-transitory computer readable medium of clause 23, wherein recommending implementation of the at least one specific solution includes selecting the at least one particular solution based on the ranking
25. The non-transitory computer readable medium of clause 21, wherein the at least one proof of concept includes information indicating how the identified at least one solution can be executed.
26. The non-transitory computer readable medium of clause 21, wherein the degree of safety includes an indication of potential harm that may arise from implementing the identified at least one solution.
27. The non-transitory computer readable medium of clause 21, wherein the economic feasibility includes a cost/benefit analysis for implementing the identified at least one solution.
28. The non-transitory computer readable medium of clause 21, wherein the commercial applicability includes an indication of how effectively the identified at least one solution can be implemented in a particular industry.
29. The non-transitory computer readable medium of clause 21, wherein the first scraping includes performing semantic analysis on scraped information.
30. The non-transitory computer readable medium of clause 21, wherein performing the second scraping includes performing semantic analysis on scraped information.
31. The non-transitory computer readable medium of clause 21, wherein performing the second scraping includes scraping a plurality of media items.
32. The non-transitory computer readable medium of clause 21, wherein the recommended implementation of at least one specific solution is further based on additional information including at least one of an indication of available resources or an expertise of a querying individual.
33. The non-transitory computer readable medium of clause 32, wherein the additional information is accessed from a database.
34. The non-transitory computer readable medium of clause 21, wherein the unmet technological need includes a resource needed for conducting a clinical trial.
35. The non-transitory computer readable medium of clause 34, wherein the resource needed for conducting the clinical trial is identified based on a least one trial protocol parameter.
36. The non-transitory computer readable medium of clause 35, wherein the resource needed for conducting the clinical trial is identified based on application of a trained machine learning model to the least one trial protocol parameter.
37. The non-transitory computer readable medium of clause 34, wherein the resource needed for conducting the clinical trial includes a specified piece of equipment and wherein the at least one specific solution includes an identification of at least one of a supplier, an operator, or a funding source for the specified piece of equipment.
38. The non-transitory computer readable medium of clause 34, wherein the resource needed for conducting the clinical trial includes an individual with a specified skillset and wherein the at least one specific solution includes an identification of at least one of the individuals having the specified skillset, a method for training an individual to obtain the specified skillset, or an individual qualified to train an individual to obtain the specified skillset.
39. A system for identifying solutions to unmet technological needs, the system comprising:
   at least one processor configured to:
      receive an indication of an unmet technological need of at least one entity;
      electronically extract data from a data source to identify a plurality of requirements for fulfilling the unmet technological need;
      perform a first scraping of the internet to identify at least one solution satisfying at least one of the plurality of requirements;
      perform a second scraping of the internet to identify at least one of:
         a proof of concept for the identified at least one solution;
         a degree of safety associated with the identified at least one solution;
         an economic feasibility associated with the identified at least one solution; and
         a commercial applicability associated with the identified at least one solution; and
      analyze the identified at least one solution, and the at least one of the scraped proof of concept, the scraped degree of safety, the scraped economic feasibility, or the scraped commercial applicability to thereby recommend implementation of at least one specific solution from the identified at least one solution.
40. A computer-implemented method for identifying solutions to unmet technological needs, the method comprising:
   receiving an indication of an unmet technological need of at least one entity;
   electronically extracting data from a data source to identify a plurality of requirements for fulfilling the unmet technological need;
   performing a first scraping of the internet to identify at least one solution satisfying at least one of the plurality of requirements;
   performing a second scraping of the internet to identify at least one of:
      a proof of concept for the identified at least one solution;
      a degree of safety associated with the identified at least one solution;
      an economic feasibility associated with the identified at least one solution; and
      a commercial applicability associated with the identified at least one solution; and
   analyzing the identified at least one solution, and the at least one of the scraped proof of concept, the scraped degree of safety, the scraped economic feasibility, or the scraped commercial applicability to thereby recommend implementation of at least one specific solution from the identified at least one solution.
41. A system for forming ephemeral social clusters, the system comprising:
   at least one processor configured to:
      scrape the internet for commonality data identifying a plurality of entities associated with a commonality;
      perform electronic semantic analysis on the scraped commonality data to identify a subset of the plurality of entities having at least one specific overlapping interest in contributing to a common purpose;
      transmit electronic communications to at least some entities of the subset of the plurality of entities;
      receive electronic responses to at least some of the electronic communications;
      based on the received responses, generate an interest group defined by the at least one specific overlapping interest;
      cause the interest group to be stored in memory, wherein the stored interest group includes information identifying selected entities from the subset of the plurality of entities;
      receive electronic data from a plurality of differing sources to thereby monitor the at least one specific overlapping interest of each of the selected entities and to determine that the overlapping interest is reduced for a specific one of the selected entities;
      and redefine the interest group to exclude the specific one of the selected entities following a determination that the at least one specific overlapping interest of the specific one of the selected entities is reduced.
42. The system of clause 41, wherein receiving electronic data from a plurality of differing sources includes scraping the internet for information about the selected entities and ascertaining reduced interest from the scraped information.
43. The system of clause 42, wherein ascertaining the reduced interest from the scraped information includes performing semantic analysis on the scraped information.
44. The system of clause 41, wherein the electronic data from a plurality of differing sources includes electronic data stored in a data structure.
45. The system of clause 44, wherein the information stored in the data structure includes status information.
46. The system of clause 41, wherein receiving electronic data from a plurality of differing sources includes scraping local data structures.
47. The system of clause 41, wherein performing electronic semantic analysis on the scraped commonality data includes performing initial semantic analysis to identify the subset of the plurality of entities, and wherein the at least one processor is further configured to perform subsequent semantic analysis to identify potentially available entities within the subset of the plurality of entities.
48. The system of clause 41, wherein the processor is further configured to subsequently scrape the internet to identify at least one new entity for inclusion within the subset of the plurality of entities.
49. The system of clause 48, wherein the at least one processor is further configured to send an additional electronic communication to the at least one new entity and, based on a response to the additional electronic communication, include the at least one new entity in the interest group.
50. The system of clause 41, wherein the at least one processor is further configured to determine completion of the common purpose and to dissolve the interest group following the determination of completion.
51. The system of clause 41, wherein with the plurality of entities are subscribers to at least one platform.
52. The system of clause 41, wherein the determination that the at least one specific overlapping interest is reduced by the specific one of the selected entities is based on a determination by the at least one processor that the specific one of the selected entities completed a role associated with the common purpose.
53. The system of clause 41, wherein the determination that the at least one specific overlapping interest is reduced by the specific one of the selected entities is based on a determination by the at least one processor that the specific one of the selected entities failed to timely complete a task associated with the common purpose.
54. The system of clause 41, wherein the at least one processor is further configured to suggest an action to the at least one selected entity.
55. The system of clause 54, wherein the suggested action includes executing at least one of an electronic nondisclosure agreement or an electronic engagement contract.
56. The system of clause 54, wherein the suggested action includes generating terms of a smart contract.
57. The system of clause 41, wherein the electronic communications include an invitation to contribute to the common purpose.
58. The system of clause 41, wherein the common purpose includes conducting a clinical trial and wherein each of the selected entities are associated with at least one skill for conducting the clinical trial.
59. A non-transitory computer readable medium for forming ephemeral social clusters, the computer readable medium containing instructions that when executed by at least one processor, cause the at least one processor to perform operations comprising:
   scraping the internet for commonality data identifying a plurality of entities associated with a commonality;
   performing electronic semantic analysis on the scraped commonality data to identify a subset of the plurality of entities having at least one specific overlapping interest in contributing to a common purpose;
   transmitting electronic communications to at least some entities of the subset of the plurality of entities;
   receiving electronic responses to at least some of the electronic communications;
   based on the received responses, generating an interest group defined by the at least one specific overlapping interest;
   causing the interest group to be stored in memory, wherein the stored interest group includes information identifying selected entities from the subset of the plurality of entities;
   receiving electronic data from a plurality of differing sources to thereby monitor the at least one specific overlapping interest of each of the selected entities and to determine that the overlapping interest is reduced for a specific one of the selected entities; and
   redefining the interest group to exclude the specific one of the selected entities following a determination that the at least one specific overlapping interest of the specific one of the selected entities is reduced.
60. A computer implemented method for forming ephemeral social clusters, the method comprising:
   scraping the internet for commonality data identifying a plurality of entities associated with a commonality;
   performing electronic semantic analysis on the scraped commonality data to identify a subset of the plurality of entities having at least one specific overlapping interest in contributing to a common purpose;
   transmitting electronic communications to at least some entities of the subset of the plurality of entities;
   receiving electronic responses to at least some of the electronic communications;
   based on the received responses, generating an interest group defined by the at least one specific overlapping interest;
   causing the interest group to be stored in memory, wherein the stored interest group includes information identifying selected entities from the subset of the plurality of entities;
   receiving electronic data from a plurality of differing sources to thereby monitor the at least one specific overlapping interest of each of the selected entities and to determine that the overlapping interest is reduced for a specific one of the selected entities; and
   redefining the interest group to exclude the specific one of the selected entities following a determination that the at least one specific overlapping interest of the specific one of the selected entities is reduced.
61. A system for associating entities with unestablished relationships in order to induce collaboration, the system comprising:
   at least one processor configured to:
      receive information identifying a current unmet technological need;
      access an electronic data source to identify, in relation to the current unmet technological need, a plurality of skill sets of a plurality of entities, and a plurality of technological need-related roles of the plurality of entities;
      scrape the internet to identify:
      at least one prior solution for resolving a previous unmet technological need, the previous unmet technological need being related to the current unmet technological need; and
      a plurality of prior roles and a plurality of prior skills associated with the at least one prior solution;
      generate at least one collaboration rule based on the identified prior solution, the identified plurality of prior roles, and the identified plurality of prior skills;
      apply the at least one collaboration rule to the electronic data source to identify, based on the plurality of skill sets and the plurality of technological need-related roles of the plurality of entities, at least two entities of the plurality of entities projected to have an ability to collaborate in order to satisfy the current unmet technological need; and
      output an identification of the at least two entities in an associative manner in connection with the current unmet technological need.
62. The system of clause 61, wherein receiving information identifying the current unmet technological need includes scraping the internet for the information identifying the current unmet technological need.
63. The system of clause 61, wherein the at least one processor is further configured to transmit at least one query for the current unmet technological need, and wherein the information identifying the current unmet technological need is received in response to the at least one query.
64. The system of clause 61, wherein accessing the electronic data source includes accessing a data structure.
65. The system of clause 61, wherein accessing the electronic data source includes scraping the internet.
66. The system of clause 61, wherein outputting the identification of the at least two entities in connection with the current unmet technological need includes providing a description of the current unmet technological need.
67. The system of clause 61, wherein outputting the identification of the at least two entities includes outputting an indication of skill sets of the at least two entities.
68. The system of clause 61, wherein outputting the identification of the at least two entities includes outputting an indication of a particular skill possessed by at least one entity of the at least one of the at least two entities.
69. The system of clause 61, wherein outputting the identification of the at least two entities includes initiating an introduction between the at least two entities.
70. The system of clause 69, wherein the at least one processor is further configured to obtain additional information regarding the at least two entities prior to initiating the introduction.
71. The system of clause 70, wherein obtaining the additional information includes running a background check on the at least two entities.
72. The system of clause 61, wherein scraping the internet to identify the at least one prior solution, the plurality of prior roles, and the plurality of prior skills includes performing semantic analysis on scraped information.
73. The system of clause 72, wherein the scraped information includes at least one of a social platform entry, a blog entry, an article, a news item, website content, or a publication.
74. The system of clause 61, wherein the current unmet technological need is associated with at least a first task and a second task, and wherein identifying the least two entities includes identifying a first entity projected to have an ability to perform the first task and a second entity projected to have an ability to perform the second task.
75. The system of clause 61, wherein the current unmet technological need is associated with a clinical trial and wherein the plurality of prior roles and the plurality of prior skills are associated with professionals involved in a previous clinical trial.
76. The system of clause 61, wherein the current unmet technological need is associated with a geographic region and wherein the at least one collaboration rule specifies that the at least two entities be associated with locations within a predetermined range of the geographic region.
77. The system of clause 61, wherein the current unmet technological need is associated with a clinical trial and the at least one entity of the at least two entities includes a medical facility.
78. The system of clause 61, wherein the collaboration rule includes a preference to minimize a quantity of the at least two entities.
79. A non-transitory computer readable medium for associating entities with unestablished relationships in order to induce collaboration, the computer readable medium containing instructions that when executed by at least one processor, cause the at least one processor to perform operations comprising:
   receiving information identifying a current unmet technological need;
   accessing an electronic data source to identify, in relation to the current unmet technological need, a plurality of skill sets of a plurality of entities, and a plurality of technological need-related roles of the plurality of entities;
   scraping the internet to identify:
   at least one prior solution for resolving a previous unmet technological need, the previous unmet technological need being related to the current unmet technological need; and
   a plurality of prior roles and a plurality of prior skills associated with the at least one prior solution;
   generating at least one collaboration rule based on the identified prior solution, the identified plurality of prior roles, and the identified plurality of prior skills;
   applying the at least one collaboration rule to the electronic data source to identify, based on the plurality of skill sets and the plurality of technological need-related roles of the plurality of entities, at least two entities of the plurality of entities projected to have an ability to collaborate in order to satisfy the current unmet technological need; and
   outputting an identification of the at least two entities in an associative manner in connection with the current unmet technological need.
80. A computer implemented method for associating entities with unestablished relationships in order to induce collaboration, the method comprising:
   receiving information identifying a current unmet technological need;
   accessing an electronic data source to identify, in relation to the current unmet technological need, a plurality of skill sets of a plurality of entities, and a plurality of technological need-related roles of the plurality of entities;
   scraping the internet to identify:
   at least one prior solution for resolving a previous unmet technological need, the previous unmet technological need being related to the current unmet technological need; and
   a plurality of prior roles and a plurality of prior skills associated with the at least one prior solution;
   generating at least one collaboration rule based on the identified prior solution, the identified plurality of prior roles, and the identified plurality of prior skills;
   applying the at least one collaboration rule to the electronic data source to identify, based on the plurality of skill sets and the plurality of technological need-related roles of the plurality of entities, at least two entities of the plurality of entities projected to have an ability to collaborate in order to satisfy the current unmet technological need; and
   outputting an identification of the at least two entities in an associative manner in connection with the current unmet technological need.
81. A system for internet-based smart contracting and collaboration, the system comprising:
   at least one processor configured to:
      access terms of a collaborative smart contract between at least two previously unconnected entities, the at least two previously unconnected entities including a first entity and a second entity respectively located in a first venue and in a second venue, the collaborative smart contract defining a plurality of success criteria for resolving an unmet technological need shared by the first entity and the second entity;
      remotely monitor, over at least one network, activity of the first entity in the first venue to track progress of the first entity in satisfying a first portion of the success criteria associated with the first entity;
      remotely monitor, over the at least one network, activity of the second entity in the second venue, to track progress in satisfying a second portion of the success criteria associated with the second entity;
      based at least on the remote monitoring of the activity of the first entity and the activity of the second entity, determine that all of the success criteria are satisfied;
      perform semantic analysis to identify a plurality of additional entities who share the unmet technological need; and
      transmit to the plurality of additional entities an indication that the success criteria are satisfied, and an opportunity related to the satisfied success criteria.
82. The system of clause 81, wherein the at least one processor is further configured to scrape the internet to identify the at least two previously unconnected entities.
83. The system of clause 82, wherein identifying the plurality of entities who share the unmet technological need further includes performing a semantic analysis on scraped information.
84. The system of clause 82, wherein the at least one processor is configured to generate the terms of the collaborative smart contract and present the collaborative smart contract to the at least two previously unconnected entities.
85. The system of clause 84, wherein the at least one processor is configured to receive and maintain executed forms of the collaborative smart contract from the at least two previously unconnected entities.
86. The system of clause 81, wherein remotely monitoring the activity of the first entity and the activity of the second entity includes periodically transmitting queries to the first entity and the second entity.
87. The system of clause 86, wherein the at least one processor is configured to generate the queries.
88. The system of clause 81, wherein the at least one processor is further configured to provide, to at least the first entity and the second entity, an invitation to enter into an additional collaborative smart contract related to the satisfied unmet technological need.
89. The system of clause 81, wherein the opportunity related to the satisfied success criteria includes an invitation to contact at least one of the first entity and the second entity.
90. The system of clause 81, wherein the opportunity related to the satisfied success criteria includes an invitation to collaborate with at least one of the first entity and the second entity.
91. The system of clause 81, wherein the opportunity related to the satisfied success criteria includes an invitation to enter into an additional collaborative smart contract with at least one of the first entity and the second entity.
92. The system of clause 91, wherein the at least one processor is further configured to generate the terms of the additional smart contract.
93. The system of clause 81, wherein remotely monitoring the activity of the first entity and the activity of the second entity includes scraping the internet to identify publications associated with the first entity and the second entity.
94. The system of clause 93, wherein remotely monitoring the activity of the first entity and the activity of the second entity further includes performing a semantic analysis on the publications.
95. The system of clause 81, wherein the collaborative smart contract is based on a blockchain network.
96. The system of clause 81, wherein the unmet technological need is associated with a clinical trial and wherein the plurality of success criteria include a plurality of activities for successfully completing the clinical trial.
97. The system of clause 96, wherein at least one activity of the plurality of activities includes identifying subjects for participation in the clinical trial.
98. The system of clause 81, wherein the at least one processor is further configured to:
   determine, based on the remote monitoring of the activity of the first entity, that at least one criterion of the first portion of the success criteria is satisfied; and
   cause a reward to be provided to the first entity based on the at least one criterion being satisfied.
99. A non-transitory computer readable medium for internet-based smart contracting and collaboration, the computer readable medium containing instructions that when executed by at least one processor, cause the at least one processor to perform operations comprising:
   accessing terms of a collaborative smart contract between at least two previously unconnected entities, the at least two previously unconnected entities including a first entity and a second entity respectively located in a first venue and in a second venue, the collaborative smart contract defining a plurality of success criteria for resolving an unmet technological need shared by the first entity and the second entity;
   remotely monitoring, over at least one network, activity of the first entity in the first venue to track progress of the first entity in satisfying a first portion of the success criteria associated with the first entity;
   remotely monitoring, over the at least one network, activity of the second entity in the second venue, to track progress in satisfying a second portion of the success criteria associated with the second entity;
   based at least on the remote monitoring of the activity of the first entity and the activity of the second entity, determining that all of the success criteria are satisfied;
   performing sematic analysis to identify a plurality of additional entities who share the unmet technological need; and
   transmitting to the plurality of additional entities an indication that the success criteria are satisfied, and an opportunity related to the satisfied success criteria.
100. A computer implemented method for internet-based smart contracting and collaboration, the method comprising:
   accessing terms of a collaborative smart contract between at least two previously unconnected entities, the at least two previously unconnected entities including a first entity and a second entity respectively located in a first venue and in a second venue, the collaborative smart contract defining a plurality of success criteria for resolving an unmet technological need shared by the first entity and the second entity;
   remotely monitoring, over at least one network, activity of the first entity in the first venue to track progress of the first entity in satisfying a first portion of the success criteria associated with the first entity;
   remotely monitoring, over the at least one network, activity of the second entity in the second venue, to track progress in satisfying a second portion of the success criteria associated with the second entity;
   based at least on the remote monitoring of the activity of the first entity and the activity of the second entity, determining that all of the success criteria are satisfied;
   performing sematic analysis to identify a plurality of additional entities who share the unmet technological need; and
   transmitting to the plurality of additional entities an indication that the success criteria are satisfied, and an opportunity related to the satisfied success criteria.
101. A system for mapping a series of related unmet technological needs and for providing resources for satisfying unmet technological needs, the system comprising:
   at least one processor configured to:
      receive first onboarding information from a plurality of first entities, the first onboarding information including information indicative of a plurality of first unmet technological needs;
      establish a map of the first unmet technological needs, the map including a plurality of nodes representing the first unmet technological needs;
      establish pathways between the plurality of nodes, wherein the pathways define relationships between nodes based on similarities between corresponding unmet technological needs;
      associate a particular entity of the plurality of first entities with a first particular node of the plurality of nodes;
      receive first content associated with the first particular node, the first content being available to the particular entity by virtue of the particular entity being associated with the first particular node;
      receive second onboarding information from a plurality of second entities, the second onboarding information including information indicative of a plurality of second unmet technological needs;
      alter the map based on the second onboarding information, wherein altering the map includes establishing a second particular node of the plurality of second unmet technological needs, establishing a pathway between the first particular node and the second particular node, and moving the particular entity from the first particular node to the second particular node, wherein the second particular node is associated with an unmet technological need different from the unmet technological need associated with the first particular node; and
      receive second content within the second particular node, the second content being available to the particular entity by virtue of the particular entity being associated with the second particular node.
102. The system of clause 101, wherein the first onboarding information further includes identifications of readiness of the plurality of first entities.
103. The system of clause 102, wherein the particular entity is associated with the first particular node based on a readiness of the particular entity.
104. The system of clause 101, wherein the first onboarding information further includes identifications of at least one of abilities of the first entities or desires of the first entities and wherein the particular entity is associated with the first particular node based on the at least one of an ability of the particular entity or a desire of the particular entity.
105. The system of clause 101, wherein receiving the first onboarding information includes scraping the internet to identify the plurality of first unmet technological needs and performing a semantic analysis on scraped information.
106. The system of clause 101, wherein receiving the first onboarding information includes querying the plurality of first entities.
107. The system of clause 101, wherein receiving the first content associated with the first particular node includes scraping the internet to determine the first content.
108. The system of clause 107, wherein scraping the internet to determine the first content includes performing semantic analysis on scraped information.
109. The system of clause 101, wherein receiving the first content within the first particular node includes accessing information posted by the plurality of first entities.
110. The system of clause 101, wherein the map is associated with a discrete stage of research of a plurality of discrete stages of research.
111. The system of clause 110, wherein the discrete stage of research is a discrete stage of medical research.
112. The system of clause 101, wherein establishing the map of the first unmet technological needs includes classifying the first unmet technological needs into a plurality of classifications and wherein each node of the plurality of nodes represents a particular classification of the plurality of classifications.
113. The system of clause 101, wherein the at least one processor is further configured to:
   identify at least one solution associated with the unmet technological need associated with the first particular node; and
   suggest the at least one solution to an entity associated with the second particular node based on the pathway between the first particular node and the second particular node.
114. The system of clause 113, wherein identifying the at least one solution includes receiving, from a solution provider, an input indicating that the solution provider can help with the unmet technological need associated with the first particular node.
115. The system of clause 113, wherein identifying the at least one solution includes scraping the internet to identify a solution provider projected to have a solution to the unmet technological need.
116. The system of clause 101, wherein the at least one processor is further configured to identify the similarities between corresponding unmet technological needs by performing a semantic analysis of the first onboarding information.
117. The system of clause 101, wherein the plurality of first unmet technological needs includes unmet medical needs of patients and wherein the similarities between corresponding unmet technological needs include at least one of a similar disease or a similar condition.
118. The system of clause 101, wherein at least one unmet technological need of the plurality of first unmet technological needs includes a regulatory requirement for a clinical trial.
119. A non-transitory computer readable medium for mapping a series of related unmet technological needs, the computer readable medium containing instructions that when executed by at least one processor, cause the at least one processor to perform operations comprising:
   receiving first onboarding information from a plurality of first entities, the first onboarding information including information indicative of a plurality of first unmet technological needs;
   establishing a map of the first unmet technological needs, the map including a plurality of nodes representing the first unmet technological needs;
   establishing pathways between the plurality of nodes, wherein the pathways define relationships between nodes based on similarities between corresponding unmet technological needs;
   associating a particular entity of the plurality of first entities with a first particular node of the plurality of nodes;
   receiving first content associated with the first particular node, the first content being available to the particular entity by virtue of the particular entity being associated with the first particular node;
   receiving second onboarding information from a plurality of second entities, the second onboarding information including information indicative of a plurality of second unmet technological needs;
   altering the map based on the second onboarding information, wherein altering the map includes establishing a second particular node of the plurality of second unmet technological needs, establishing a pathway between the first particular node and the second particular node, and moving the particular entity from the first particular node to the second particular node, wherein the second particular node is associated with an unmet technological need different from the unmet technological need associated with the first particular node; and
   receiving second content within the second particular node, the second content being available to the particular entity by virtue of the particular entity being associated with the second particular node.
120. A computer implemented method for mapping a series of related unmet technological needs, the method comprising:
   receiving first onboarding information from a plurality of first entities, the first onboarding information including information indicative of a plurality of first unmet technological needs;
   establishing a map of the first unmet technological needs, the map including a plurality of nodes representing the first unmet technological needs;
   establishing pathways between the plurality of nodes, wherein the pathways define relationships between nodes based on similarities between corresponding unmet technological needs;
   associating a particular entity of the plurality of first entities with a first particular node of the plurality of nodes;
   receiving first content associated with the first particular node, the first content being available to the particular entity by virtue of the particular entity being associated with the first particular node;
   receiving second onboarding information from a plurality of second entities, the second onboarding information including information indicative of a plurality of second unmet technological needs;
   altering the map based on the second onboarding information, wherein altering the map includes establishing a second particular node of the plurality of second unmet technological needs, establishing a pathway between the first particular node and the second particular node, and moving the particular entity from the first particular node to the second particular node, wherein the second particular node is associated with an unmet technological need different from the unmet technological need associated with the first particular node; and
   receiving second content within the second particular node, the second content being available to the particular entity by virtue of the particular entity being associated with the second particular node.

Systems and methods disclosed herein involve unconventional improvements over conventional approaches. Descriptions of the disclosed embodiments are not exhaustive and are not limited to the precise forms or embodiments disclosed. Modifications and adaptations of the embodiments will be apparent from consideration of the specification and practice of the disclosed embodiments. Additionally, the disclosed embodiments are not limited to the examples discussed herein.

The foregoing description has been presented for purposes of illustration. It is not exhaustive and is not limited to the precise forms or embodiments disclosed. Modifications and adaptations of the embodiments will be apparent from consideration of the specification and practice of the disclosed embodiments. For example, the described implementations include hardware and software, but systems and methods consistent with the present disclosure may be implemented as hardware alone.

Computer programs based on the written description and methods of this specification are within the skill of a software developer. The various functions, scripts, programs, or modules may be created using a variety of programming techniques. For example, programs, scripts, functions, program sections or program modules may be designed in or by means of languages, including JAVASCRIPT, C, C++, JAVA, PHP, PYTHON, RUBY, PERL, BASH, or other programming or scripting languages. One or more of such software sections or modules may be integrated into a computer system, non-transitory computer readable media, or existing communications software. The programs, modules, or code may also be implemented or replicated as firmware or circuit logic.

Moreover, while illustrative embodiments have been described herein, the scope may include any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as non-exclusive. Further, the steps of the disclosed methods may be modified in any manner, including by reordering steps or inserting or deleting steps. It is intended, therefore, that the specification and examples be considered as exemplary only, with a true scope and spirit being indicated by the following claims and their full scope of equivalents.

1-40. (canceled)

41. A system for forming ephemeral social clusters, the system comprising:
at least one processor configured to:
scrape the internet for commonality data identifying a plurality of entities associated with a commonality;
perform electronic semantic analysis on the scraped commonality data to identify a subset of the plurality of entities having at least one specific overlapping interest in contributing to a common purpose;
transmit electronic communications to at least some entities of the subset of the plurality of entities;
receive electronic responses to at least some of the electronic communications;
based on the received responses, generate an interest group defined by the at least one specific overlapping interest;
cause the interest group to be stored in memory, wherein the stored interest group includes information identifying selected entities from the subset of the plurality of entities;
receive electronic data from a plurality of differing sources to thereby monitor the at least one specific overlapping interest of each of the selected entities and to determine that the overlapping interest is reduced for a specific one of the selected entities; and
redefine the interest group to exclude the specific one of the selected entities following a determination that the at least one specific overlapping interest of the specific one of the selected entities is reduced.

42. The system of claim 41, wherein receiving electronic data from a plurality of differing sources includes scraping the internet for information about the selected entities and ascertaining reduced interest from the scraped information.

43. The system of claim 42, wherein ascertaining the reduced interest from the scraped information includes performing semantic analysis on the scraped information.

44. The system of claim 41, wherein the electronic data from a plurality of differing sources includes electronic data stored in a data structure.

45. The system of claim 44, wherein the information stored in the data structure includes status information.

46. The system of claim 41, wherein receiving electronic data from a plurality of differing sources includes scraping local data structures.

47. The system of claim 41, wherein performing electronic semantic analysis on the scraped commonality data includes performing initial semantic analysis to identify the subset of the plurality of entities, and wherein the at least one processor is further configured to perform subsequent semantic analysis to identify potentially available entities within the subset of the plurality of entities.

48. The system of claim 41, wherein the processor is further configured to subsequently scrape the internet to identify at least one new entity for inclusion within the subset of the plurality of entities.

49. The system of claim 48, wherein the at least one processor is further configured to send an additional electronic communication to the at least one new entity and, based on a response to the additional electronic communication, include the at least one new entity in the interest group.

50. The system of claim 41, wherein the at least one processor is further configured to determine completion of the common purpose and to dissolve the interest group following the determination of completion.

51. The system of claim 41, wherein with the plurality of entities are subscribers to at least one platform.

52. The system of claim 41, wherein the determination that the at least one specific overlapping interest is reduced by the specific one of the selected entities is based on a determination by the at least one processor that the specific one of the selected entities completed a role associated with the common purpose.

53. The system of claim 41, wherein the determination that the at least one specific overlapping interest is reduced by the specific one of the selected entities is based on a determination by the at least one processor that the specific one of the selected entities failed to timely complete a task associated with the common purpose.

54. The system of claim 41, wherein the at least one processor is further configured to suggest an action to the at least one selected entity.

55. The system of claim 54, wherein the suggested action includes executing at least one of an electronic nondisclosure agreement or an electronic engagement contract.

56. The system of claim 54, wherein the suggested action includes generating terms of a smart contract.

57. The system of claim 41, wherein the electronic communications include an invitation to contribute to the common purpose.

58. The system of claim 41, wherein the common purpose includes conducting a clinical trial and wherein each of the selected entities are associated with at least one skill for conducting the clinical trial.

59. A non-transitory computer readable medium for forming ephemeral social clusters, the computer readable medium containing instructions that when executed by at least one processor, cause the at least one processor to perform operations comprising:
scraping the internet for commonality data identifying a plurality of entities associated with a commonality;
performing electronic semantic analysis on the scraped commonality data to identify a subset of the plurality of entities having at least one specific overlapping interest in contributing to a common purpose;
transmitting electronic communications to at least some entities of the subset of the plurality of entities;
receiving electronic responses to at least some of the electronic communications;
based on the received responses, generating an interest group defined by the at least one specific overlapping interest;
causing the interest group to be stored in memory, wherein the stored interest group includes information identifying selected entities from the subset of the plurality of entities;
receiving electronic data from a plurality of differing sources to thereby monitor the at least one specific overlapping interest of each of the selected entities and to determine that the overlapping interest is reduced for a specific one of the selected entities; and
redefining the interest group to exclude the specific one of the selected entities following a determination that the at least one specific overlapping interest of the specific one of the selected entities is reduced.

60. A computer implemented method for forming ephemeral social clusters, the method comprising:
scraping the internet for commonality data identifying a plurality of entities associated with a commonality;
performing electronic semantic analysis on the scraped commonality data to identify a subset of the plurality of entities having at least one specific overlapping interest in contributing to a common purpose;
transmitting electronic communications to at least some entities of the subset of the plurality of entities;
receiving electronic responses to at least some of the electronic communications;
based on the received responses, generating an interest group defined by the at least one specific overlapping interest;
causing the interest group to be stored in memory, wherein the stored interest group includes information identifying selected entities from the subset of the plurality of entities;
receiving electronic data from a plurality of differing sources to thereby monitor the at least one specific overlapping interest of each of the selected entities and to determine that the overlapping interest is reduced for a specific one of the selected entities; and
redefining the interest group to exclude the specific one of the selected entities following a determination that the at least one specific overlapping interest of the specific one of the selected entities is reduced.

61-120. (canceled)
